SEQUENCE LISTING (192 sequences)

SEQ ID NO: 1 - protein, 190 aa, Moraxella bovoculi
MNNLKKTAITHDGVFAYKNTETVIGSVGRNDIVMAIDATHGEFNDKNFIIYADTNGNPIYLGYAYLDDNNDAHIDLAVGACNEDDDFDEKEIHEMIAEQMELAKRYQELGDTVHGTTRLAFDDDGYMTVRLDQQAYPDYRPENDDKHIMWRALALTATGKELEVFWLVEDYEDEEVNSWDFDIADDWREL

SEQ ID NO: 2 - protein, 100 aa, Pseudomonas aeruginosa
MSIQVTSTNGRTVNLEIELGSVVASSGQVKFMADKTDRGLESRFLVPEAGNRRIEVALTGRDLEAANALFSELAASVEATNEMYRELDAERAQINKALEG

SEQ ID NO: 3 - protein, 57 aa, Pseudomonas aeruginosa
MDNKITPADEEKIREWLNCEEASVDNDGDVWVAVPMTGHWLSDEQKAKYIEWRGDET

SEQ ID NO: 4 - protein, 60 aa, Pseudomonas aeruginosa
MSKVTLNGQQIDFDAAVNLMDAELREELHSAQEWTNDQEFLDAYVQAHAAKFDGEEFQVA

SEQ ID NO: 5 - protein, 144 aa, Pseudomonas aeruginosa
MTESLIHLRVPAATKGRWVRASRAVGLRLTDYITQAVEAYMQQQLTRVAIPDDIEFSDLKLARDPDGAVSFDWAVIERICHASGLPLEMMRDAPEDNVASLIIGWYQAHRADGGAADPVADDLIAEAMAEDAAGQQFSHQPGRA

SEQ ID NO: 6 - protein, 94 aa, Pseudomonas aeruginosa
MATVTKITLNGQNHYNFGSECSEADAEGYREWIAQELAENFPGAEIEINEADSTYSVVVEIDDESYYDEARGLKDDVNVFCIDAWDRCPWDWVS

SEQ ID NO: 7 - protein, 80 aa, Pseudomonas aeruginosa
MYAIRKIQFFYGPTDKKSYVGEEAGGRRELFKTRAEAQARIEDLEEGVYYLAHNESGRPDYKIVWVRGEAQFEHARWMRG

SEQ ID NO: 8 - protein, 98 aa, Sulfolobus islandicus
MNYKELEKMLDVIFENSEIKEIDLFFDPEVEISKQEFEDLVKNADPLQKVVGDNYITETFEWWEFENQYLEFELDYYVKDEKIFVLEMHFWRKIRKLE

SEQ ID NO: 9 - protein, 100 aa, Pseudomonas aeruginosa
MEKKLSDAQVALVAAWRKYPDLRESLEEAASILSLIVFQAETLSDQANELANYIRRQGLEEAEGACRNIDIMRAKWVEVCGEVNQHGIRVYGDAIDRDVD

SEQ ID NO: 10 - protein, 84 aa, Pseudomonas aeruginosa
MNTYLIDPRKNNDNSGERFTVDAVDITAAAKSAAQQILGEEFEGLVYRETGESNGSGMFQAYHHLHGTNRTETTVGYPFHVMEL

SEQ ID NO: 11 - protein, 68 aa, Pseudomonas aeruginosa
MKITNDTTTYEVAELMGSEADELDGRIMMGLLSRECVVDTDDLSEDQWLALIDESQKVRREQFESDEA

SEQ ID NO: 12 - protein, 52 aa, Pseudomonas aeruginosa
MSTQYTYEQIAEDFRLWGEYMDPNAEMTEEEFQALSTEEKVAMQVEAFGAEA

SEQ ID NO: 13 - protein, 65 aa, Pseudomonas aeruginosa [sequence omitted]

SEQ ID NO: 14 - protein, 79 aa, Pseudomonas aeruginosa
MNNDTEVLEQQIKAFELLADELKDRLPTLEILSPMYTAVMVTYDLIGKQLASRRAELIEILEEQYPGHAADLSIKNLCP

SEQ ID NO: 15 - protein, 106 aa, Pseudomonas aeruginosa
MIGSEKQVNWAKSIIEKEVEAWEAIGVDVREVAAFLRSISDARVIIDNRNLIHFQSSGISYSLESSPLNSPIFLRRFSACSVGFEEIPTALQRIRSVYTAKLLEDE

SEQ ID NO: 16 - protein, 119 aa, Pseudomonas aeruginosa
MSTQYTYQQIAEDFRLWSEYVDTAGEMSKDEFNSLSTEDKVRLQVEAFGEEKSPKFSTKVTTKPDFDGFQFYIEAGRDFDGDAYTEAYGVAVPTNIAARIQAQAAELNAGEWLLVEHEA

SEQ ID NO: 17 - protein, 78 aa, Pseudomonas aeruginosa
MKFIKYLSTAHLNYMNIAVYENGSKIKARVENVVNGKSVGARDFDSTEQLESWFYGLPGSGLGRIENAMNEISRRENP

SEQ ID NO: 18 - protein, 90 aa, Pseudomonas aeruginosa
MIAQQHKDTVAACEAAEAIAIAKDQVWDGEGYTKYTFDDNSVLIQSGTTQYAMDADDADSIKGYADWLDDEARSAEASEIERLLESVEEE

SEQ ID NO: 19 - protein, 140 aa, Pseudomonas aeruginosa
MSSTISDRIISRSVIEAARFIQSWEDADPDNLTESQVLAASSFAARLHEGLQATVLQRLVDESNRDEYREFQAWEEALLNADGRVTSNPFADWGWWYRIANVMLATASQNVGVAWGSHVHGRLMAIFQDRFQQHYEDEEC

SEQ ID NO: 20 - protein, 100 aa, Pseudomonas aeruginosa
MMTISKTDIDCYLQTYVVIDPVSNGWQWGIDENGVGGALHHGRVEMVEGENGYFGLRGATHPTEKEAMAAALGYLWKCRQDLVAIARNDAIEAEKYRAKA

SEQ ID NO: 21 - protein, 79 aa, Pseudomonas aeruginosa
MSRPTVVTVTETPRNPGSYEVNVERDGKMVVGRARAGSDPGAAAAKAMQMAMEWGSPNYVILGSNKVLAFIPEQLRVKM

SEQ ID NO: 22 - protein, 100 aa, Pseudomonas aeruginosa
MKVPAFFAANILTIEQIIEAINNDGSAMTSAPEIAGYYAWDAATDALESENDLEQLTEDDFVAHLEVLEERGAKIDRDAAIAVALQFQAAAVNDLHSGDE

SEQ ID NO: 23 - protein, 83 aa, Pseudomonas aeruginosa
MSHASHNGEAPKRIEAMTTFTSIVTTNPDFGGFEFYVEAGQQFDDSAYEEAYGVSVPSAVVEEMNAKAAQLKDGEWLNVSHEA

SEQ ID NO: 24 - protein, 92 aa, Pectobacterium phage Z40
MARIAPNEDSTMSTAYIIFNSSVAAVVDTEIANGANVTFSTVTVKEEINANRDFNLVNAQNGKISRAKRWGNEASKCEYFGREINPTEFFIK

SEQ ID NO: 25 - protein, 68 aa, Vibrio parahaemolyticus
MKAAYIIKEVQNINSEREGTQIEATSLSQAKRIASKEQCFHGTVMRIETVNGLWLAYKEDGKRWVDCQ

SEQ ID NO: 26 - protein, 97 aa, Shewanella xiamenensis
MTTFRIENVRIETINDFDMVKFDLVTDLGRVELAEHVNYDSEGDFKSVEYTDSNIRYNMVDELCSVFDLTDKPSLMPAIDYVTFAEIIEAVEEMLEA

SEQ ID NO: 27 - protein, 132 aa, Pseudomonas aeruginosa
MSMELFHGSYEEISEIRDSGVFGGLFGAHEKETALSHGETLHRIISPLPLTDYALNYEIESAWEVALDVAGGDENVAEAIMAKACESDSNDGWELQRLRGVLAVRLGYTSVEMEDEHGTTWLCLPGCTVEKI

SEQ ID NO: 28 - protein, 124 aa, Pseudomonas aeruginosa
MAYEKTWHRDYAAESLKRAETSRWTQDANLEWTQLALECAQVVHLARQVGEELGNEKIIGIADTVLSTIEAHSQATYRRPCYKRITTAQTHLLAVTLLERFGSARRVANAVWQLTDDEIDQAKA

SEQ ID NO: 29 - protein, 115 aa, Moraxella catarrhalis
MKLLNIKINEFAVTANTEAGDELYLQLPHTPDSQHSINHEPLDDDDFVKEVQEICDEYFGKGDRTLARLSYAGGQAYDSYTEEDGVYTTNTGDQFVEHSYADYYNVEVYCKADLV

SEQ ID NO: 30 - protein, 124 aa, Moraxella catarrhalis
MKKIEMIEISQNRQNLTAFLHISEIKAINAKLADGVDVDKKSFDEICSIVLEQYQAKQISNKQASEIFETLAKANKSFKIEKFRCSHGYNEIYKYSPDHEAYLFYCKGGQGQLNKLIAENGRFM

SEQ ID NO: 31 - protein, 119 aa, Pseudomonas aeruginosa; identical to SEQ ID NO: 16

SEQ ID NO: 32 - protein, 149 aa, Listeria monocytogenes
MTIKLLDEFLKKHDLTRYQLSKLTGISQNTLKDQNEKPLNKYTVSILRSLSLISGLSVSDVLFELEDIEKNSDDLAGFKHLLDKYKLSFPAQEFELYCLIKEFESANIEVLPFTFNRFENEEHVNIKKDVCKALENAITVLKEKKNELL

SEQ ID NO: 33 - protein, 123 aa, Listeria monocytogenes
MTLTRAQKKYAEAMHEFINMVDDFEESTPDFAKEVLHDSDYVVITKNEKYAVALCSLSTDECEYDTNLYLDEKLVDYSTVDVNGVTYYINIVETNDIDDLEIATDEDEMKSGNQEIILKSELK

SEQ ID NO: 34 - protein, 125 aa, Listeria monocytogenes
MFNKAEIMKQAWNWFNDSNIWLSDIEWVSYTDKEKSFSVCLKAAWSKAKEEVEESKKESKHIAKSEELKAWNWAERKLGLHFNISDDEKFTSVKDETKINFGLSVWACAMKAVKLHNDLFPQTAA

SEQ ID NO: 35 - protein, 87 aa, Listeria monocytogenes
MNINDLIREIKNKDYTVKLSGTDSNSITQLIIRVNNDGNEYVISESENESIVEKFISAFKNGWNQEYEDEEEFYNDMQTITLKSELN

SEQ ID NO: 36 - protein, 140 aa, Streptococcus thermophilus
MAYGKSRYNSYRKRSFNRSNKQRREYAQEMDRLEKAFENLDGWYLSSMKDSAYKDFGKYEIRLSNHSADNKYHDLENGRLIVNIKASKLNFVDIIENKLDKIIEKIDKLDLDKYRFINATNLEHDIKCYYKGFKTKKEVI

SEQ ID NO: 37 - protein, 183 aa, Streptococcus thermophilus
MKINDDIKELILEYMSRYFKFENDFYKLPGIKFTDANWQKFKNGGTDIEKMGAARVNAMLDCLFDDFELAMIGKAQTNYYNDNSLKMNMPFYTYYDMFKKQQLLKWLKNNRDDVIGGTGRMYTASGNYIANAYLEVALESSSLGSGSYMLQMRFKDYSKGQEPIPSGRQNRLEWIENNLENIR

SEQ ID NO: 38 - protein, 103 aa, unknown
  Obtained from meta sequencing data
MTFGQALESLKRGHLVARKGWNGKGMFIFMRPEDSLPTNMIVNQVKSLPESFKRWVANNHGDSETDRIKFTAYLCMKAADGTIVNGWLASQTDMLANDWVIVE

SEQ ID NO: 39 - protein, 105 aa, unknown
  Obtained from meta sequencing data
MSIFTDMIPAELLINEYKKGQSGAKHDNYVSVGRIMVAIYKNNSFKNTGTVKYQDSTHSGITMSKVFIDGKEYRIDIDTQHYEVQDFDTSGRQTTLILKRIDLYG

SEQ ID NO: 40 - protein, 141 aa, unknown
  Obtained from meta sequencing data
MKGTEHFKQTIKEYLDGRAQTDELFAVSYAKENKNLDDCITFILNQVKASGCCGMTDDEVWSLAIHYYDEDNIDVGNPISCGVVVNHKVELTEEEKAQARKEALKAYQEEEMRKIQQRHSKPKPTAKAAQSNQTELSLFDF

SEQ ID NO: 41 - protein, 109 aa, unknown
  Obtained from meta sequencing data
MDNKFKLRKAINGIEELNFAFDKLTAIDYKTICRIERKMNGLSVDALADSIIASAGTRKTSSEFRIACAWVAAVKGTDGLTVDDYDQLSLDDLLELETFGLLFFVGSLE

SEQ ID NO: 42 - protein, 182 aa, unknown
  Obtained from meta sequencing data
MADMTLRQFCERYRKGDFLAKDRETQIEAGWYDWFCDDKALAGRLAKIWGILKGITSDYILDNYRVWFKNNCPMVGPLYDDVRFEPLDEEQRDELYFGVAIDDKRREKKYVIFTARNDYENECGFNNVREVRQFINGWEDELKNEEFYKAREKKRQEMEEANNKFAEIMQRADEILWNLKED

SEQ ID NO: 43 - protein, 83 aa, Listeria monocytogenes
MSKTMYKNDVIELIKNAKTNNEELLFTSVERNTREAATQYFRCPEKHVSDAGVYYGEDFEFDGFEIFEDDLIYTRSYDKEELN

SEQ ID NO: 44 - protein, 129 aa, Staphylococcus schleiferi
MEVMNKSIEIKDQNNIVLIDSLGQFFTDIENDNNGRYNIDYVLLNEVEHDNGNTYYEVGMYRTEEVPFSDKVTQDNVELLEDKWLQIDQQGESYVESIFFENEEDAREYIKLVLKGHETFEETAKAIGV

SEQ ID NO: 45 - protein, 159 aa, unknown
  Synthetic Sequence
LKKTIEKLLNSDLNSNYIAKKTGVEQSTIYRLRTGERQLGKLGLDSAERLYNYQKEIENMKSVKYISNMSKQEKGYRVYVNVVNEDTDKGFLFPSVPKEVIENDKIDELFNFEHHKPYVQKAKSRYDKNGIGYKIVQLDEGFQKFIELNKEKMKENLDY

SEQ ID NO: 46 - protein, 170 aa, Staphylococcus delphini
MRKTIERLLNSELSSNSIAVRTGVSQAVISKLRNGKKELGNLTLNSAEKLFEYQKEMEKVDTWIVYRGRTADMNKSYIAEGSTYEEVYNNFVDKYGYDVLDEDIYEIQLLKKNGENLDDYDVDSDGINNYDKLDEFRESDYVDLEDYDYRELFENSSSQVYYHEFEITHE

SEQ ID NO: 47 - protein, 202 aa, Listeria monocytogenes
MGYIGTKRSERSQDAIEDYEVPLNHFNKDLIQAFIDENEAYDTLKTKKVRLWKFVAPRAGATSWHHTGTYYNKTDHYSLEKVADELLQNGDEWEEQFKAYVKEEQETATSEPVFLSVIKVQIWGGSMKRPKLVGHEVVMGVKKEGWLHAVSKATQSKYKLSANKVEMQKHYSLEDYSALTKDFPEFKAQKRAINKKMKEMYN

SEQ ID NO: 48 - protein, 204 aa, Enterococcus faecalis
MGYVGKSRSVRSQIAIDNAEVPLNHITKDYILTFVTENNIDETLKNESVAMWKFVAKRHGSTSWHHVSKHYNKIDHYDLHDVAEYFSMNYDSLKNDYQNLLDQKRQAKNDLIKNLKLGIIKVQIWGGTKRYPKLEGYESVMGVVKDGWLHTVTLSNQTKYKITGNKIEEITIFELDQYDILTKKFPEFRAMKRKINKEVARLSK

SEQ ID NO: 49 - protein, 109 aa, Enterococcus faecalis
MAILNNKGEKISIDCADLISEVEEDILIFGGTFLVYAICSWREIEQVEYISDYVHADNPESYKDELTTKEYAELKEIYEKDLEELKITKNKQMNLNELLSILTIQNSIT

SEQ ID NO: 50 - protein, 100 aa, Streptococcus gallolyticus
MKISVDSEKLLNEAINDFDIFGEDFNVYAIYSYREDYDFEYISDYVDADEPTRDEFETEEDYQEVMKDFKENLDSLKFTKHKKMTIADLVHELWEQNRIF

SEQ ID NO: 51 - protein, 182 aa, Streptococcus macedonicus
MKIDTTVTEVKENGKTYLRLLKGNEQLKAVSDKAVAGVNLFPGAKIGSFLVRQDNIVVFPDNKGEFDLDFFNLLNDNFETLVEYAKMADCLDIAFDINEKSYFNMIMWLMKNIDENWSQSPYGESFYSSKDIDWGYKPEGSLRVSDHWNFGQDGEHCPTAEPVDGWAVCKFENGKYHLIKKF

SEQ ID NO: 52 - protein, 181 aa, Streptococcus gallolyticus
MKIDTTVTEVKENGKTYLRLVEGTEQLKAISDKAMAGVNLFPGAKIDSFLVKQDSIVVFPDNKGEFDLDFFKQLDENFDTIAKYARVATCFEEVAFDEKSYFNMIMWLMDNMDENWSQSPYGESFYSSKNIDWGYKPEGSLRVSDHWNFGENGEHCPTAEPVDGWAVCKFENGKYHLIKKF

SEQ ID NO: 53 - protein, 124 aa, Staphylococcus simulans
MKLIVEVEETNYKNLVNYTKLTNESHNILVNRLISEYITKPYELRLDLSERYSNRDLIEFKFMLIEYCKEALQDIKELANSDEAYETDEAFEAVFRQLFEEVISNPDTVLKAFHSYTSFLEENK

SEQ ID NO: 54 - protein, 115 aa, Staphylococcus pseudintermedius
MKLIINIEDKNYKYLTELAQQDNTNIGSIVNNLIQTHITDVNESYRSVDKKELDEFSRVMQHYFHEDLASMYDVIGSDEELSTDKQMLKVYKKLYQDVALRNGIALELFNAYKKG

SEQ ID NO: 55 - protein, 64 aa, unknown
  Synthetic Sequence
MKNYEVTNEVKNLNTQVETIGQAVDLYKEYGSNTIVWSIDKNEDLIDEVTELVAEYAEKGTVIK

SEQ ID NO: 56 - protein, 108 aa, unknown
  Synthetic Sequence
MDYDNENYLIPKILLQDDFYSSLSAKDILVYAVLKDRQIEALEKGWIDTDGSIYLNFKLIELAKMFSCSRTTMIDVMQRLEEVNLIERERVDVFYGYSLPYKTYINEV

SEQ ID NO: 57 - protein, 86 aa, Brackiella oedipodis
MANKTYKIGKNAGYDGCGLCLAAISENEAIKVKYLRDICPDYDGDDKAEDWLRWGTDSRVKAAALEMEQYAYTSVGMASCWEFVEL

SEQ ID NO: 58 - protein, 123 aa, Neisseria meningitidis
MSKNNIFNKYPTIIHGEARGENDEFVVHTRYPRFLARKSFDDNFTGEMPAKPVNGELGQIGEPRRLAYDSRLGLWLSDFIMLDNNKPKNMEDWLGQLKAACDRIAADDLMLNEDAADLEGWDD

SEQ ID NO: 59 - protein, 116 aa, Neisseria meningitidis
MFKRAIIFTSFNGFEKVSRTEKRRLAKIINARVSIIDEYLRAKDTNASLDGQYRAFLFNDESPAMTEFLAKLKAFAESCTGISIDAWEIEESEYVRLPVERRDFLAAANGKEIFKI

SEQ ID NO: 60 - protein, 88 aa, Haemophilus parainfluenzae
MKITSSNFATIATSENFAKLSVLPKNHREPIKGLFKSAVEQFSSARDFFKNENYSKELAEKFNKEAVNEAVEKLQKAIDLAEKQGIQF

SEQ ID NO: 61 - protein, 130 aa, Simonsiella muelleri
MNNSIKFHVSYDGTARALFNTKEQAEKYCLVEEINDEMNGYKRKSWEEKLREENCASVQDWVEKNYTSSYSDLFNICEIEVSSAGQLVKIDNTEVDDFVENCYGFTLEDDLEEFNKAKQYLQKFYAECEN

SEQ ID NO: 62 - protein, 144 aa, unknown; identical to SEQ ID NO: 5
  Synthetic Sequence

SEQ ID NO: 63 - protein, 94 aa, unknown; identical to SEQ ID NO: 6
  Synthetic Sequence

SEQ ID NO: 64 - protein, 80 aa, unknown; identical to SEQ ID NO: 7
  Synthetic Sequence

SEQ ID NO: 65 - protein, 114 aa, unknown
  Synthetic Sequence
MNKVYLANAFSINMLTKFPTKVVIDKIDRLEFCENIDNEDIINSIGHDSTIQLINSLCGTTFQKNRVEIKLEKEDKLYVVQISQRLEEGKILTLEEILKLYESGKVQFFEIIVD

SEQ ID NO: 66 - protein, 249 aa, Sulfolobus islandicus
MEVKQIKKLNNLPWVFLDTYLNKFALDKNFVNCAYYSSRSGMTQEGCVQVMQVGDNFKVDTMREVHGIYFTPHASIISLIYRQKGIRSIDDLKEILGSLNLSKVSPKHYQLLVKYSNYTIEIYDIYFKGHIYEFPLVSQQGHLNVYNVPEPRNVYLIYYENNEEKKELNKDLFNEVSEFMIYNHRVTFEKPVLEFKNLQITPGGGALVYVPESMYVKLESSDHQSVEFRPSRDDWLLFSHPRPRRSGND

SEQ ID NO: 67 - protein, 170 aa, Moraxella bovoculi
MYEAKERYAKKKMQENTKIDTLTDEQHDALAQLCAFRHKFHSNKDSLFLSESAFSGEFSFEMQSDENSKLREVGLPTIEWSFYDNSHIPDDSFREWFNFANYSELSETIQEQGLELDLDDDETYELVYDELYTEAMGEYEELNQDIEKYLRRIDEEHGTQYCPTGFARLR

SEQ ID NO: 68 - protein, 322 aa, Moraxella bovoculi
MHHTIARMNAFNKAFANAKDCYKKMQAWHLLNKPKHAFFPMQNTPALDNGLAALYELRGGKEDAHILSILSRLYLYGAWRNTLGIYQLDEEIIKDCKELPDDTPTSIFLNLPDWCVYVDISSAQIATFDDGVAKHIKGFWAIYDIVEMNGINHDVLDFVVDTDTDDNVYVPQPFILSSGQSVAEVLDYGASLFDDDTSNTLIKGLLPYLLWLCVAEPDITYKGLPVSREELTRPKHSINKKTGAFVTPSEPFIYQIGERLGSEVRRYQSIIDGEQKRNRPHTKRPHIRRGHWHGYWQGTGQAKEFRVRWQPAVFVNSGRVSS

SEQ ID NO: 69 - protein, 168 aa, Moraxella bovoculi
MVGKSKIDWQSIDWTKTNAQIAQECGRAYNTVCKMRGKLGKSHQGAKSPRKDKGISRPQPHLNRLEYQALATAKAKASPKAGRFETNTKAKTWTLKSPDNKTYTFTNLMHFVRTNPHLFDPDDVVWRTKSNGVEWCRASSGLALLAKRKKAPLSWKGWRLISLTKDNK

SEQ ID NO: 70 - protein, 234 aa, Moraxella bovoculi
MYEIKLNDTLIHQTDDRVNAFVAYRYLLRRGDLPKCENIARMYYDGKVIKTDVIDHDSVHSDEQAKVSNNDIIKMAISELGVNNFKSLIKKQGYPFSNGHINSWFTDDPVKSKTMHNDEMYLVVQALIRACIIKEIDLYTEQLYNIIKSLPYDKRPNVVYSDQPLDPNNLDLSEPELWAEQVGECMRYAHNDQPCFYIGSTKRELRVNYIVPVIGVRDEIERVMTLEEVRNLHK

SEQ ID NO: 71 - protein, 92 aa, Moraxella bovoculi
MKIELSGGYICYSIEEDEVTIDMVEVTTKRQGIGSQLIDMVKDVAREVGLPIGLYAYPQDDSISQEDLIEFYFSNDFEYDPDDVDGRLMRWS

SEQ ID NO: 72 - protein, 74 aa, Leptotrichia wadei
MEKIKLICLRINNDELITTDKDEWLKFIKRHRGKVSSIEQFNWKIPGNKLQKALEYSFDELYKFKQKENRRETD

SEQ ID NO: 73 - protein, 77 aa, Leptotrichia wadei
MWKCKKCGCDRFYQDITGGISEVLEMDKDGEVLDEIDDVEYGDFSCAKCDNSSSKIQEIAYWDEINGKNKTYLSKDK

SEQ ID NO: 74 - protein, 79 aa, Leptotrichia wadei
MFKEFLEKCLRYGNLYILEETGDRKKVKRISKRHGKVTEASVLLFDSGTKRTTINEIYLNSQGYFIIRDQKRLKLEKFK

SEQ ID NO: 75 - protein, 76 aa, Leptotrichia wadei
MDKANRCLKAKDKILNILEKEEITLDEFNNISKDIAKEYVEKAVLKPKDIAERIINMVKNAKSISFDELASEISEE

SEQ ID NO: 76 - protein, 124 aa, Leptotrichia wadei
MERNFKKVTENTGRKEVFKVMHDKVEIINDFNTNEKREARIIFHDQKIYVILYQNLNFEELKWLNFYILIYGNQSYGKNTFFEFKLNKNNLIYHLQVWNIIENKKFKSKSISLLVKALSSKAGV

SEQ ID NO: 77 - protein, 85 aa, Leptotrichia wadei
MADKVKSIQPGPIFYDVFLVYLRVIGTNLKDWCAPHGVTATNAKSAATGGWNGTKARALRQKMIDEVGEETFLRLYTERLRREAA

SEQ ID NO: 78 - protein, 151 aa, Leptotrichia wadei
MRIIKLYERIIPKTSSTSYISRWEALNIPDENRNTAAWHPRTYLFSYDKDKAINLYNTTNVLGNSGIKKRIIDYPSKREVYIANFPRAIADLVLTMKDYQLSSLHNCCNDFFNEDETEQLYQYLRSIKDNRRVDEFLKYEFTVRYFNDKKF

SEQ ID NO: 79 - protein, 232 aa, Listeria seeligeri
MIYYIKDLKVKGKIFENLMNKEAVEGLITFLKKAEFEIYSRENYSKYNKWFEMWKSPTSSLVFWKNYSFRCHLLFVIEKDGECLGIPASVFESVLQIYLADPFAPDTKELFVEVCNLYECLADVTVVEHFEAEESAWHKLTHNETEVSKRVYSKDDDELLKYIPEFLDTIATNKKSQKYNQIQGKIQEINKEIATLYESSEDYIFTEYVSNLYRESAKLEQHSKQILKEELN

SEQ ID NO: 80 - protein, 170 aa, Artificial sequence; identical to SEQ ID NO: 67
  Synthetic Sequence

SEQ ID NO: 81 - protein, 87 aa, Artificial sequence
  Synthetic Sequence
MNINDLIREIKNKDYTVKLSGTDSNSITQLIIRVNNDGAEYVISESENESIVEKFISAFKNGWNQEYEDEEEFYNDMQTITLKSELN

SEQ ID NO: 82 - protein, 87 aa, Artificial sequence
  Synthetic Sequence
MNINDLIREIKNKAYTVKLSGTDSNSITQLIIRVNNDANEYVISESENESIVEKFISAFKNGWNQEYEDEEEFYNDMQTITLKSELN

SEQ ID NO: 83 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 84 - protein, 1368 aa, Streptococcus pyogenes [sequence omitted]

SEQ ID NO: 85 - protein, 1081 aa, Neisseria meningitidis
MAAFKPNPINYILGLDIGIASVGWAMVEIDEDENPICLIDLGVRVFERAEVPKTGDSLAAARRLARSVRRLTRRRAHRLLRARRLLKREGVLQAADFDENGLIKSLPNTPWQLRSAALDRKLTPLEWSAVLLHLIKHRGYLSQRKNEGETADKELGALLKGVADNAHALQTGDFRTPAELALNKFEKESGHIRNRRGDYSHTFSRKDLQAELDLLFEKQKEFGNPHISDDLKEGIETLLMTQRPALSGDAVQKMLGHCTFEPTEPKAAKNTYTAERFVWLTKLNNLRILEQGSERPLTDTERATLMDEPYRKSKLTYAQARKLLELDDTAFFKGLRYGKDNAEASTLMEMKAYHAISRALEKEGLKDKKSPLNLSPELQDEIGTAFSLFKTDEDITGRLKDRVQPEILEVLLKHISFDKFVQISLKALRRIVPLMEQGKRYDEACAEIYGDHDGKKNTEEKIYLPPIPADEIRNPVVLRALSQARKVINAVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHMLLTGKGKRRVFASNGQITNLLRGFWGLRKVRAENNRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKAGKYYLVPIYSWQVAKGILPDRAVVAYADEEGWTVIDESFRFKFVLYSNDLIKVQLKKDSFLGYFSGLDRATGAISLREHDLEKSKGKDGMHRIGVKTALSFQKYQIDEMGKEIRLCRLKKRPPVR

SEQ ID NO: 86 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 87 - protein, 7 aa, Artificial sequence
  Synthetic Sequence
PKKKRKV

SEQ ID NO: 88 - protein, 16 aa, Artificial sequence
  Synthetic Sequence
KRPAATKKAGQAKKKK

SEQ ID NO: 89 - protein, 9 aa, Artificial sequence
  Synthetic Sequence
PAAKRVKLD

SEQ ID NO: 90 - protein, 11 aa, Artificial sequence
  Synthetic Sequence
RQRRNELKRSP

SEQ ID NO: 91 - protein, 38 aa, Artificial sequence
  Synthetic Sequence
NQSSNFGPMKGGNFGGRSSGPYGGGGQYFAKPRNQGGY

SEQ ID NO: 92 - protein, 42 aa, Artificial sequence
  Synthetic Sequence
RMRIZFKNKGKDTAELRRRRVEVSVELRKAKKDEQILKRRNV

SEQ ID NO: 93 - protein, 8 aa, Artificial sequence
  Synthetic Sequence
VSRKRPRP

SEQ ID NO: 94 - protein, 8 aa, Artificial sequence
  Synthetic Sequence
PPKKARED

SEQ ID NO: 95 - protein, 8 aa, Artificial sequence
  Synthetic Sequence
PQPKKKPL

SEQ ID NO: 96 - protein, 12 aa, Artificial sequence
  Synthetic Sequence
SALIKKKKKMAP

SEQ ID NO: 97 - protein, 5 aa, Artificial sequence
  Synthetic Sequence
DRLRR

SEQ ID NO: 98 - protein, 7 aa, Artificial sequence
  Synthetic Sequence
PKQKKRK

SEQ ID NO: 99 - protein, 10 aa, Artificial sequence
  Synthetic Sequence
RKLKKKIKKL

SEQ ID NO: 100 - protein, 10 aa, Artificial sequence
  Synthetic Sequence
REKKKFLKRR

SEQ ID NO: 101 - protein, 20 aa, Artificial sequence
  Synthetic Sequence
KRKGDEVDGVDEVAKKKSKK

SEQ ID NO: 102 - protein, 17 aa, Artificial sequence
  Synthetic Sequence
RKCLQAGMNLEARKTKK

SEQ ID NO: 103 - protein, 11 aa, Artificial sequence
  Synthetic Sequence
YGRKKRRQRRR

SEQ ID NO: 104 - protein, 12 aa, Artificial sequence
  Synthetic Sequence
RRQRRTSKLMKR

SEQ ID NO: 105 - protein, 27 aa, Artificial sequence
  Synthetic Sequence
GWTLNSAGYLLGKINLKALAALAKKIL

SEQ ID NO: 106 - protein, 33 aa, Artificial sequence
  Synthetic Sequence
KALAWEAKLAKALAKALAKHLAKALAKALKCEA

SEQ ID NO: 107 - protein, 16 aa, Artificial sequence
  Synthetic Sequence
RQIKIWFQNRRMKWKK

SEQ ID NO: 108 - protein, 11 aa, Artificial sequence
  Synthetic Sequence
YGRKKRRQRRR

SEQ ID NO: 109 - protein, 9 aa, Artificial sequence
  Synthetic Sequence
RKKRRQRRR

SEQ ID NO: 110 - protein, 11 aa, Artificial sequence
  Synthetic Sequence
YGRKKRRQRRR

SEQ ID NO: 111 - protein, 8 aa, Artificial sequence
  Synthetic Sequence
RKKRRQRR

SEQ ID NO: 112 - protein, 11 aa, Artificial sequence
  Synthetic Sequence
YARAAARQARA

SEQ ID NO: 113 - protein, 11 aa, Artificial sequence
  Synthetic Sequence
THRLPRRRRRR

SEQ ID NO: 114 - protein, 11 aa, Artificial sequence
  Synthetic Sequence
GGRRARRRRRR

SEQ ID NO: 115 - protein, 5 aa, Artificial sequence
  Synthetic Sequence
GSGGS

SEQ ID NO: 116 - protein, 6 aa, Artificial sequence
  Synthetic Sequence
GGSGGS

SEQ ID NO: 117 - protein, 4 aa, Artificial sequence
  Synthetic Sequence
GGGS

SEQ ID NO: 118 - protein, 4 aa, Artificial sequence
  Synthetic Sequence
GGSG

SEQ ID NO: 119 - protein, 5 aa, Artificial sequence
  Synthetic Sequence
GGSGG

SEQ ID NO: 120 - protein, 5 aa, Artificial sequence
  Synthetic Sequence
GSGSG

SEQ ID NO: 121 - protein, 5 aa, Artificial sequence
  Synthetic Sequence
GSGGG

SEQ ID NO: 122 - protein, 5 aa, Artificial sequence
  Synthetic Sequence
GGGSG

SEQ ID NO: 123 - protein, 5 aa, Artificial sequence
  Synthetic Sequence
GSSSG

SEQ ID NO: 124 - DNA, 22 bp, Artificial sequence
  Synthetic Sequence
cgatcctgagacttccacactg

SEQ ID NO: 125 - DNA, 22 bp, Artificial sequence
  Synthetic Sequence
ccaatctactcccaggagcagg

SEQ ID NO: 126 - DNA, 22 bp, Artificial sequence
  Synthetic Sequence
cccatgtggagagacaaaagga

SEQ ID NO: 127 - DNA, 22 bp, Artificial sequence
  Synthetic Sequence
cttaaaccaacctgctcactgg

SEQ ID NO: 128 - DNA, 21 bp, Artificial sequence
  Synthetic Sequence
agccagggctgggcataaaag

SEQ ID NO: 129 - DNA, 23 bp, Artificial sequence
  Synthetic Sequence
ccaataggcagagagagtcagtg

SEQ ID NO: 130 - DNA, 20 bp, Artificial sequence
  Synthetic Sequence
taaaaggcagggcagagtcg

SEQ ID NO: 131 - DNA, 19 bp, Artificial sequence
  Synthetic Sequence
gaggttgtccaggtgagcc

SEQ ID NO: 132 - DNA, 20 bp, Artificial sequence
  Synthetic Sequence
gtgaacgtggatgaagttgg

SEQ ID NO: 133 - protein, 19 aa, Artificial sequence
  Synthetic Sequence
ATNFSLLKQAGDVEENPGP

SEQ ID NO: 134 - protein, 20 aa, Artificial sequence
  Synthetic Sequence
QCTNYALLKLAGDVESNPGP

SEQ ID NO: 135 - protein, 22 aa, Artificial sequence
  Synthetic Sequence
VKQTLNFDLLKLAGDVESNPGP

SEQ ID NO: 136 - protein, 18 aa, Artificial sequence
  Synthetic Sequence
EGRGSLLTCGDVEENPGP

SEQ ID NO: 137 - protein, 42 aa, Artificial sequence
  Synthetic Sequence
QCTNYALLKLAGDVESNPGPVKQTLNFDLLKLAGDVESNPGP

SEQ ID NO: 138 - protein, 60 aa, Artificial sequence
  Synthetic Sequence
EGRGSLLTCGDVEENPGPQCTNYALLKLAGDVESNPGPVKQTLNFDLLKLAGDVESNPGP

SEQ ID NO: 139 - DNA, 587 bp, Artificial sequence
  Synthetic Sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 140 - DNA, 587 bp, Artificial sequence
  Synthetic Sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatctggccacaacc

SEQ ID NO: 141 - DNA, 587 bp, Artificial sequence
  Synthetic Sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacggtgataatgtggccacaacc

SEQ ID NO: 142 - DNA, 587 bp, Artificial sequence
  Synthetic Sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgacgataatacggccacaacc

SEQ ID NO: 143 - DNA, 587 bp, Artificial sequence
  Synthetic Sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatttggccacaacc

SEQ ID NO: 144 - DNA, 221 bp, Artificial sequence
  Synthetic Sequence
ggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctg

SEQ ID NO: 145 - DNA, 273 bp, Artificial sequence
  Synthetic Sequence
agcactggccgtggccgtggggcccgcggagggcggcgcgcggctgatgaaaccggctcggattccgcccgcgtgcgccatcccctcagctagcaggtgtgagcggctttctgcccgcagtctctacacagctcagcatcctgacgcctcctccccttgcaggggcgtgaagctacttcagactctgctgtgacgacttggccgccaggcaccgatcctccccggtgagaaggtccacgaatcttactgcagacagatttgctcagcgcgatg

SEQ ID NO: 146 - DNA, 352 bp, Artificial sequence
  Synthetic Sequence
aattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgct

SEQ ID NO: 147 - DNA, 368 bp, Artificial sequence
  Synthetic Sequence
ctcccctgtgaggaactactgtcttcacgcagaaagcgtctagccatggcgttagtatgagtgtcgtgcagcctccaggcccccccctcccgggagagccatagtggtctgcggaaccggtgagtacaccggaattgccaggacgaccgggtcctttcttggatcaatcccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacaaatcctaaacctcaaagaaaaaccaaacgtaacaccaaccgccgcccacaggacgt

SEQ ID NO: 148 - DNA, 570 bp, Artificial sequence
  Synthetic Sequence
gacctcgtgaaataaaagtgcagaaaacaaacccaggcgatcacagcagcagccgccgcggcagcagcaccaacagcaggaggagcaggaggagccggaggaggaggaggaggaggaggcaaagttagagttggggctggcgctccggagttgctgggctcagcgcagctcccattcattaaggaaccagctgcggaggaaggtggccgagcgcccgcgctgcccactcgctcgctcgcgcactcagacgcgcgccacaacagcgcgccccaagctgcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgccgcgcccggagccggggccgacccgccactatccgcagcagcctcggccaggaggcgacccgggcgcctgggtgtgtggctgctgttgcgggacgtcttcgcggggcgggaggctcgcgccgcagccagcgccat

SEQ ID NO: 149 - DNA, 580 bp, Artificial sequence
  Synthetic Sequence
aagaagaggtagcgagtggacgtgactgctctatcccgggcaaaagggatagaaccagaggtggggagtctgggcagtcggcgacccgcgaagacttgaggtgccgcagcggcatccggagtagcgccgggctccctccggggtgcagccgccgtcgggggaagggcgccacaggccgggaagacctcctccctttgtgtccagtagtggggtccaccggagggcggcccgtgggccgggcctcaccgcggcgctccgggactgtggggtcaggctgcgttgggtggacgcccacctcgccaaccttcggaggtccctgggggtcttcgtgcgccccggggctgcagagatccaggggaggcgcctgtgaggcccggacctgccccggggcgaagggtatgtggcgagacagagccctgcacccctaattcccggtggaaaactcctgttgccgtttccctccaccggcctggagtctcccagtcttgtcccggcagtgccgccctccccactaagacctaggcgcaaaggcttggctcatggttgacagctcagagagagaaagatctgagggaagatg

SEQ ID NO: 150 - DNA, 588 bp, Artificial sequence
  Synthetic Sequence
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 151 - DNA, 611 bp, Artificial sequence
  Synthetic Sequence
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatcttgccacaaccccgggagatgaggatcgtttcgc

SEQ ID NO: 152 - DNA, 621 bp, Artificial sequence
  Synthetic Sequence
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatcatg

SEQ ID NO: 153 - DNA, 652 bp, Artificial sequence
  Synthetic Sequence
cagagtaatgacatggttccttccatcctccaaaggtgaccaataatagtttgtaagtatcattatgaactaatgaattttcaacatatttgatatatttcaatccattgccatcattgttcttatcgatatttgagttggctcactttgccagtaagagtctattcaaattggcttctgagtccatttgacacaacacctttgatctttgacagtttccttggttttaggtgctagatgatttctcaggctcaccttagacatttcctgccacagacttagaatcagccatttctctaaggaccctgattccatttcatgagaaatgatagagaccacaatcaaaacaagtcatgaatttatactgatattttcaattcaaattaaagatgaggtttttgctaaatttttttgagtttatatttgtatgtcttatgctgaaaaatcttgtttcctaattagtaacataattattcatttgatgggtaaatattttagggccgattctttggttttatagccaagataccctgttgataaagtcttgtgggagcaattataagactggcttattttgaagctttttaaaaaagacatccttacctgttttaactgtagattatattaacttaaataggtacagcccacgctt

SEQ ID NO: 154 - DNA, 586 bp, Artificial sequence
  Synthetic Sequence
gcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaa

SEQ ID NO: 155 - DNA, 1011 bp, Artificial sequence
  Synthetic Sequence
acatgagagggggagaaataaatatacagtgcttgtccttagcctttctgtgggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcaaagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggtta

SEQ ID NO: 156 - DNA, 58 bp, Artificial sequence
  Synthetic Sequence
gaattccctcctcctcctcctcctccgaattcggaggaggaggaggaggagggaattc

SEQ ID NO: 157 - DNA, 98 bp, Artificial sequence
  Synthetic Sequence
gaattccctcctcctcctcctcctcccctcctcctcctcctcctccgaattcggaggaggaggaggaggaggggaggaggaggaggaggagggaattc

SEQ ID NO: 158 - DNA, 80 bp, Artificial sequence
  Synthetic Sequence
gaaagaaagaaagaaagaaagaaagaaactgcagccgcgcctgcagaaagaaagaaagaaagaaagaaagaaagaaagaa

SEQ ID NO: 159 - DNA, 92 bp, Artificial sequence
  Synthetic Sequence
aaaagaaggaaaaagaaggaaaagaaggaaaaagaaggctgcaggcggctgcagaaaagaaggaaaaagaaggaaaagaaggaaaaagaagg

SEQ ID NO: 160 - DNA, 10 bp, Artificial sequence
  Synthetic Sequence
gccrccatgg

SEQ ID NO: 161 - protein, 90 aa, Artificial sequence
  Synthetic Sequence
MRNLKELVREIEKKGYRVINQTTDLVIDINGNGADYPIKANHYKSILEQFIEIFKNGWNGVYEDEETFYNDMQEIAKNIVLENLEVTYDS

SEQ ID NO: 162 - DNA, 9450 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccggatccgccaccatgtatgaggccaaggagaggtacgccaagaagaagatgcaggagaacaccaagatcgataccctgacagacgagcagcacgatgcactggcacagctgtgcgccttcagacacaagtttcactccaataaggactctctgttcctgagcgagtccgcctttagcggcgagttctcctttgagatgcagtctgacgagaacagcaagctgagggaagtgggactgccaaccatcgagtggtccttctatgataattctcacatccctgacgatagctttagagagtggttcaactttgccaattactctgagctgagcgagacaatccaggagcagggcctggagctggacctggacgatgacgagacctacgagctggtgtatgatgagctgtacacagaggccatgggcgagtatgaggagctgaaccaggacatcgagaagtacctgcggagaatcgatgaggagcacggcacccagtattgtcccacaggcttcgccaggctgcgctgagaattcgaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 163 - DNA, 9183 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaacatcaatgacctgatccgggagatcaagaacaaggattacaccgtgaagctgagcggcacagacagcaattccatcacccagctgatcatcagagtgaacaatgatggcgccgagtatgtgatctctgagagcgagaatgagtccatcgtggagaagttcatctctgcctttaagaacggctggaatcaggagtacgaggacgaggaggagttctataacgatatgcagaccatcacactgaagtccgagctgaattgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 164 - DNA, 9192 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaggaaccttaaagaactggtgcgggagatcgaaaagaaagggtatagagtgatcaaccaaacgactgacctcgtaattgacatcaatggcaatggagccgactatcccattaaagcaaaccactacaagtccatcttagaacagttcatcgagatttttaagaatggttggaatggcgtgtacgaagatgaggagacattctataacgatatgcaggaaatagctaagaatatagtcttggagaacctggaggttacctacgatagctaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 165 - DNA, 9183 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaacatcaatgacctgatccgggagatcaagaacaaggattacaccgtgaagctgagcggcacagacagcaattccatcacccagctgatcatcagagtgaacaatgatggcaacgagtatgtgatctctgagagcgagaatgagtccatcgtggagaagttcatctctgcctttaagaacggctggaatcaggagtacgaggacgaggaggagttctataacgatatgcagaccatcacactgaagtccgagctgaattgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 166 - DNA, 39 bp, Artificial sequence
  Synthetic Sequence
ttttttcaggttggaccggtgccaccatgaggaacctta

SEQ ID NO: 167 - DNA, 41 bp, Artificial sequence
  Synthetic Sequence
ttttttcaggttggaccggtgccaccatgaacatcaatgac

SEQ ID NO: 168 - DNA, 20 bp, Artificial sequence
  Synthetic Sequence
gaattctaactagagctcgc

SEQ ID NO: 169 - DNA, 40 bp, Artificial sequence
  Synthetic Sequence
atgataatatggccacaaccgactataaggaccacgacgg

SEQ ID NO: 170 - DNA, 21 bp, Artificial sequence
  Synthetic Sequence
cccctctccctcccccccccc

SEQ ID NO: 171 - DNA, 40 bp, Artificial sequence
  Synthetic Sequence
gggggggggagggagaggggctagctatcgtaggtaacct

SEQ ID NO: 172 - DNA, 40 bp, Artificial sequence
  Synthetic Sequence
gggggggggagggagaggggtcaattcagctcggacttca

SEQ ID NO: 173 - DNA, 25 bp, Artificial sequence
  Synthetic Sequence
accggtccaacctgaaaaaaagtga

SEQ ID NO: 174 - DNA, 40 bp, Artificial sequence
  Synthetic Sequence
gcgagctctagttagaattctcactttttcttttttgcct

SEQ ID NO: 175 - DNA, 20 bp, Artificial sequence
  Synthetic Sequence
ggttgtggccatattatcat

SEQ ID NO: 176 - DNA, 319 bp, Artificial sequence
  Synthetic Sequence
ttttttcaggttggaccggtgccaccatgaggaaccttaaagaactggtgcgggagatcgaaaagaaagggtatagagtgatcaaccaaacgactgacctcgtaattgacatcaatggcaatggagccgactatcccattaaagcaaaccactacaagtccatcttagaacagttcatcgagatttttaagaatggttggaatggcgtgtacgaagatgaggagacattctataacgatatgcaggaaatagctaagaatatagtcttggagaacctggaggttacctacgatagctagcccctctccctccccccccc

SEQ ID NO: 177 - DNA, 310 bp, Artificial sequence
  Synthetic Sequence
ttttttcaggttggaccggtgccaccatgaacatcaatgacctgatccgggagatcaagaacaaggattacaccgtgaagctgagcggcacagacagcaattccatcacccagctgatcatcagagtgaacaatgatggcaacgagtatgtgatctctgagagcgagaatgagtccatcgtggagaagttcatctctgcctttaagaacggctggaatcaggagtacgaggacgaggaggagttctataacgatatgcagaccatcacactgaagtccgagctgaattgacccctctccctccccccccc

SEQ ID NO: 178 - DNA, 4079 bp, Artificial sequence
  Synthetic Sequence
gaattctaactagagctcgctgatcagcctcgactgtgccttctagttgccagccatctgttgtttgccccaccggtccaacctgaaaaaaagtgatcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggt

SEQ ID NO: 179 - DNA, 4309 bp, Artificial sequence
  Synthetic Sequence
atgataatatggccacaaccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagaattctaactagagctcgc

SEQ ID NO: 180 - DNA, 587 bp, Artificial sequence; identical to SEQ ID NO: 139
  Synthetic Sequence

SEQ ID NO: 181 - DNA, 9264 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatacgcgttgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctctggctaactagagaacccactgcttactggcttatcgaaattaatacgactcactatagggagacccaagctggctagcgtttaaacttaagcttggtaccgagctcggatccgccaccttgaacatcaatgacctgatccgggagatcaagaacaaggattacaccgtgaagctgagcggcacagacagcaattccatcacccagctgatcatcagagtgaacaatgatggcaacgagtatgtgatctctgagagcgagaatgagtccatcgtggagaagttcatctctgcctttaagaacggctggaatcaggagtacgaggacgaggaggagttctataacgatatgcagaccatcacactgaagtccgagctgaattgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 182 - DNA, 9264 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatacgcgttgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctctggctaactagagaacccactgcttactggcttatcgaaattaatacgactcactatagggagacccaagctggctagcgtttaaacttaagcttggtaccgagctcggatccgccaccctgaacatcaatgacctgatccgggagatcaagaacaaggattacaccgtgaagctgagcggcacagacagcaattccatcacccagctgatcatcagagtgaacaatgatggcaacgagtatgtgatctctgagagcgagaatgagtccatcgtggagaagttcatctctgcctttaagaacggctggaatcaggagtacgaggacgaggaggagttctataacgatatgcagaccatcacactgaagtccgagctgaattgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 183 - DNA, 9264 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatacgcgttgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctctggctaactagagaacccactgcttactggcttatcgaaattaatacgactcactatagggagacccaagctggctagcgtttaaacttaagcttggtaccgagctcggatccgccaccatgaacatcaatgacctgatccgggagatcaagaacaaggattacaccgtgaagctgagcggcacagacagcaattccatcacccagctgatcatcagagtgaacaatgatggcaacgagtatgtgatctctgagagcgagaatgagtccatcgtggagaagttcatctctgcctttaagaacggctggaatcaggagtacgaggacgaggaggagttctataacgatatgcagaccatcacactgaagtccgagctgaattgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 184 - DNA, 9513 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatacgcgttgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctctggctaactagagaacccactgcttactggcttatcgaaattaatacgactcactatagggagacccaagctggctagcgtttaaacttaagcttggtaccgagctcggatccgccaccatgtatgaggccaaggagaggtacgccaagaagaagatgcaggagaacaccaagatcgataccctgacagacgagcagcacgatgcactggcacagctgtgcgccttcagacacaagtttcactccaataaggactctctgttcctgagcgagtccgcctttagcggcgagttctcctttgagatgcagtctgacgagaacagcaagctgagggaagtgggactgccaaccatcgagtggtccttctatgataattctcacatccctgacgatagctttagagagtggttcaactttgccaattactctgagctgagcgagacaatccaggagcagggcctggagctggacctggacgatgacgagacctacgagctggtgtatgatgagctgtacacagaggccatgggcgagtatgaggagctgaaccaggacatcgagaagtacctgcggagaatcgatgaggagcacggcacccagtattgtcccacaggcttcgccaggctgcgctgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 185 - DNA, 9273 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatacgcgttgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctctggctaactagagaacccactgcttactggcttatcgaaattaatacgactcactatagggagacccaagctggctagcgtttaaacttaagcttggtaccgagctcggatccgccaccctgaggaaccttaaagaactggtgcgggagatcgaaaagaaagggtatagagtgatcaaccaaacgactgacctcgtaattgacatcaatggcaatggagccgactatcccattaaagcaaaccactacaagtccatcttagaacagttcatcgagatttttaagaatggttggaatggcgtgtacgaagatgaggagacattctataacgatatgcaggaaatagctaagaatatagtcttggagaacctggaggttacctacgatagctaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 186 - DNA, 9273 bp, Artificial sequence
  Synthetic Sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgaacgtggatgaagttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatacgcgttgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctctggctaactagagaacccactgcttactggcttatcgaaattaatacgactcactatagggagacccaagctggctagcgtttaaacttaagcttggtaccgagctcggatccgccaccatgaggaaccttaaagaactggtgcgggagatcgaaaagaaagggtatagagtgatcaaccaaacgactgacctcgtaattgacatcaatggcaatggagccgactatcccattaaagcaaaccactacaagtccatcttagaacagttcatcgagatttttaagaatggttggaatggcgtgtacgaagatgaggagacattctataacgatatgcaggaaatagctaagaatatagtcttggagaacctggaggttacctacgatagctaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 187 - DNA, 36 bp, Artificial sequence
  Synthetic Sequence
gccaccctgaatatcaacgatctcattcgggagatc

SEQ ID NO: 188 - DNA, 36 bp, Artificial sequence
  Synthetic Sequence
gccaccgtgaatatcaacgatctcattcgggagatc

SEQ ID NO: 189 - DNA, 36 bp, Artificial sequence
  Synthetic Sequence
gccaccacgaatatcaacgatctcattcgggagatc

SEQ ID NO: 190 - DNA, 36 bp, Artificial sequence
  Synthetic Sequence
gccaccataaatatcaacgatctcattcgggagatc

SEQ ID NO: 191 - DNA, 36 bp, Artificial sequence
  Synthetic Sequence
gccaccttgaatatcaacgatctcattcgggagatc

SEQ ID NO: 192 - DNA, 36 bp, Artificial Sequence
  Synthetic Sequence
gccaccatgaacatcaatgacctgatccgggagatc

(19) United States
(12) Patent Application Publication
(10) Pub. No.: US 2023/0374502 A1
(43) Pub. Date: Nov. 23, 2023

(54) COMPOSITIONS AND METHODS FOR ENHANCED NUCLEIC ACID TARGETING SPECIFICITY

(71) Applicant: Acrigen Biosciences, Inc., Kensington, CA (US)

(72) Inventors: David Rabuka, Kensington, CA (US); Michael Schelle, San Francisco, CA (US); Luisa Mayumi Arake de Tacca, Albany, CA (US)

(21) Appl. No.: 18/027,723

(22) PCT Filed: Sep. 30, 2021
(86) PCT No.: PCT/US2021/052933

§ 371 Date: Mar. 22, 2023

Related U.S. Application Data
(60) Provisional application No. 63/086,974, filed on Oct. 2, 2020.
(60) Provisional application No. 63/086,976, filed on Oct. 2, 2020.
(60) Provisional application No. 63/086,992, filed on Oct. 2, 2020.

(51) Int. Cl. C12N 15/11; C12N 9/22; C12N 15/85

(52) U.S. Cl. CPC C12N 15/111; C12N 9/22; C12N 15/85; C12N 2310/20; C12N 2840/203

(57) ABSTRACT

Provided are compositions and methods that utilize Acr proteins with CRISPR Cas proteins to achieve a balance in which Cas proteins retain activity to perform on-target nucleic acid targeting functions (e.g., DNA cleavage for gene editing applications), but are inhibited by one or more Acr proteins to a degree that decreases off-target activity—thus resulting in an increased ratio of on-target to off-target nucleic acid targeting events. For example, provided is a system that includes one or more nucleic acids that comprise: a first nucleotide sequence encoding a Cas effector protein and a second nucleotide sequence encoding an Acr protein, wherein the first, second or both nucleotide sequences are operably linked to a translational control element.

### CROSS REFERENCE

This application claims benefit of U.S. Provisional Patent Application Nos. 63/086,974, filed Oct. 2, 2020, 63/086,976, filed Oct. 2, 2020, and 63/086,992, filed Oct. 2, 2020, which applications are incorporated herein by reference in their entirety.

### INCORPORATION BY REFERENCE OF SEQUENCE LISTING

### Provided as a Text File

A Sequence Listing is provided herewith as a text file, “ACRG-002WO_SeqList_ST25.txt” created on Sep. 29, 2021 and having a size of 294 KB. The contents of the text file are incorporated by reference herein in their entirety

### I. INTRODUCTION

CRISPR (clustered, regularly interspaced, short palindromic repeats)-Cas systems are found in diverse bacterial and archaeal species, serving as an immune defense mechanism against phage infection. The simplicity, programmability, and versatility of Class 2 CRISPR-Cas systems (e.g., Cas9 and Cas12 systems) have facilitated the genetic modification of many organisms and offer immense therapeutic potential for the treatment of human disease. However, in practice CRISPR-Cas mediated genome editing is associated with off-targeting, e.g., introduction of unintended mutations, insertions, or deletions, and DNA restructuring at unintended “off-target” sites. Off-target editing caused by CRISPR-Cas systems has been reported in various cell and animal models, including in human cells, and such events might accumulate in vivo with prolonged nuclease activity. Further, human genetic variations and uncertain Cas protein expression lifetimes in vivo add to the unpredictability of off-target events, which should be addressed for safe clinical translation of CRISPR-Cas systems. Unintended editing events can potentially lead to genomic instability, disrupt the functionality of genes, and cause serious adverse events including cell death and cancer.

There is a need for compositions and methods that decrease off-target events (e.g., relative to on-target events), thus leading to enhanced nucleic acid targeting specificity. The present disclosure provides such compositions and methods.

### II. SUMMARY

Recently, proteins referred to as anti-CRISPR (Acr) proteins were discovered in phages. These Acr proteins bind to and inhibit certain Cas proteins, thwarting the CRISPR system's attempt to cleave invading phage DNA. Thus, Acr proteins are apparently used by phage to evade CRISPR-Cas systems.

The present disclosure provides compositions and methods that utilize Acr proteins in combination with CRISPR Cas proteins outside of their naturally-occurring context to achieve a balance in which Cas proteins retain sufficient activity to perform a desired on-target nucleic acid functions (e.g., loading, complex assembling, binding and cleavage), but are inhibited to a degree that decreases off-target activity. Thus, the compositions and methods disclosed herein combine one or more Acr proteins with one or more Cas proteins in a ratio relative to one another that results in an increase of the ratio of on-target to off-target nucleic acid targeting of a CRISPR complex. The present disclosure provides compositions and methods for delivering one or more Acr proteins with one or more Cas proteins in a coordinated delivery system at a ratio relative to one another such that the ratio of on-target to off-target nucleic acid targeting (of the CRISPR complex) is enhanced relative to the ratio of on-target to off-target in the absence of the Acr protein. For example, when the one or more Acr proteins is delivered together with the one or more Cas proteins (i.e., the one or more Acr proteins are not delivered after but are instead delivered with the one or more Cas proteins), off-target events are reduced, but the CRISPR complex retains on-target function (e.g., DNA cleavage function).

The present disclosure includes a coordinated delivery system for the expression of the Acr protein and Cas nuclease. In one embodiment, the coordinated delivery system provides one or more nucleic acids that include: (a) a first nucleotide sequence encoding a CRIPSR-associated (Cas) protein (e.g., a Cas effector protein), (b) a second nucleotide sequence encoding an anti-CRISPR protein (Acr protein), wherein the Acr protein is an inhibitor of the Cas effector protein, and (c) a translational control element that regulates translation of the Cas effector protein or the Acr protein, thereby modulating activity of the Cas effector protein. In other words, the sequence encoding the Cas protein or the Acr protein is operably linked to that translational control element. The Acr protein is an inhibitor of the Cas protein, and the translational control element can provide for an expression ratio of the Acr protein to the Cas protein in a host cell sufficient to increase the ratio of on-target to off-target nucleic acid activity (e.g., cleavage) by a CRISPR complex relative to the ratio of on-target to off-target nucleic acid activity of said CRISPR complex in the absence of the Acr protein. The coordinated delivery system retains a sufficient level of on-target nucleic acid activity such that the desired or intended outcome (e.g., nucleic acid editing) is accomplished and the CRISPR complex activity is not completely inhibited (i.e., at least some detectable activity is retained).

In some cases, the first (Cas-encoding) and second (Acr-encoding) nucleotide sequences are positioned in tandem (one of the sequences upstream of the other sequence), are operably linked to the same promoter, and a translational control element is positioned between them. In some such cases the first nucleotide sequence is positioned 5′ of the second nucleotide sequence and in other cases, the second nucleotide sequence is positioned 5′ of the first nucleotide sequence.

In some embodiments, the translational control element encodes one or more 2A peptides (e.g., P2A, F2A, E2A, T2A, or any combination thereof). In some cases, the translational control element encodes 2 or more 2A peptides in tandem to each other (e.g., in some cases 2, 3, 4, or 5 2A peptides in tandem). In some cases, at least one of the one or more 2A peptides comprise an amino acid sequence set forth in any one of SEQ ID Nos. 133-138.

In some embodiments, the translational control element comprises an IRES sequence. In some cases, an IRES sequence comprises a nucleic acid sequence set forth in any one of SEQ ID Nos. 139-159. In some cases, an IRES sequence is selected from the group consisting of the following IRES sequences: EMCV, BIP, CAT-1, c-myc, HCV, VCIP, Apaf-1, mEMCV-1, mEMCV-2, HRV, NRF, FGF-1, KMI1, KM12, (GAAA)16, (PPT19)4, EMCV mutant 5, EMCV mutant 10, EMCV mutant 15, and EMCV mutant 21.

In some cases, the first and second nucleotide sequences are operably linked to different and/or separate promoters. In some such cases, a spacer-encoding sequence is positioned 5′ of the first (Cas-encoding) nucleotide sequence and is operably linked to the same promoter—where the translational control element is positioned between the spacer encoding sequence and the first nucleotide sequence. Likewise, in some cases a spacer-encoding sequence is positioned 5′ of the second (Acr-encoding) nucleotide sequence and is operably linked to the same promoter—where the translational control element is positioned between the spacer encoding sequence and the second nucleotide sequence. In some embodiments where the first and second nucleotide sequences are operably linked to different and/or separate promoters, the first nucleotide sequence and second nucleotide sequence are carried on a single host-compatible vector. In some embodiments, the first nucleotide sequence and second nucleotide sequence are carried on separate host-compatible vectors.

In some cases, the first nucleotide sequences is operably linked to a first promoter and the second nucleotide sequences is operably linked to a second promoter such that the Acr and Cas encoding sequences are transcribed as separate RNAs. In some such cases the first and second promoters are different from one another and in other cases they are the same (i.e., the first and second promoters are copies of one another).

In some embodiments, the translational control element is a non-AUG start codon. In some cases, the non-AUG start codon is used as the initiation codon for the Acr encoding sequence (i.e., the non-AUG start codon is in frame with and 5′ of the Acr encoding sequence). In some such cases, the sequence encoding the Acr protein does not include the native AUG start codon (e.g., the non-AUG start codon replaces the native AUG).

In some cases, the non-AUG start codon is used as the initiation codon for the Cas encoding sequence (i.e., the non-AUG start codon is in frame with and 5′ of the Cas encoding sequence). In some such cases the sequence encoding the Cas protein (e.g., Cas effector protein) does not include the native AUG start codon (e.g., the non-AUG start codon replaces the native AUG).

In some cases, a non-AUG start codon (used with the Acr sequence or with the Cas sequence) is any one of: CUG, GUG, ACG, AUA, UUG, GCG, AGG, AAG, AUC, or AUU (e.g., in some cases CUG, GUG, ACG, AUA, or UUG). In some cases, a non-AUG start codon (used with the Acr sequence or with the Cas sequence) is GUG.

In some cases, the coordinated delivery system includes a single host-compatible vector to express both the Acr protein and the Cas protein. In some cases, the coordinated delivery system provides 2 or more vectors for the Acr protein and the Cas protein (e.g., one for each protein). Host compatible vectors include vectors for expression in any convenient organism, e.g., for expression in any eukaryotic cell such as for insect expression, plant expression and animal expression. For example, host compatible vectors can include vectors for microbial expression, insect expression, plant expression and animal expression. In some cases, the vector is a viral vector. In some cases, the vector(s) is a viral vector compatible with an animal, such as a mammalian host (e.g., an AAV, lentivirus, adenovirus, and the like).

The coordinated delivery system provided herein includes a selected Cas protein (e.g., a nuclease) and an Acr protein that inhibits the selected Cas protein. In some cases, the Cas protein is a Cas 9 protein, and the Acr protein is selected from the group consisting of: AcrIIA1, AcrIIA2, AcrIIA3, AcrIIA4, AcrIIA5, AcrIIA6, AcrIIA7, AcrIIA8, AcrIIA9, AcrIIA10, AcrIIA11, AcrIIA12, AcrIIA13, AcrIIA14, AcrIIA15, AcrIIA16, AcrIIA17, AcrIIA18, and AcrIIA19. In some such cases the Cas 9 protein is NmeCas9 and the Acr protein is selected from the group consisting of Acr-IIC1, Acr-IIC2, Acr-IIC3, Acr-IIC4, and Acr-IIC5. In some cases, the Cas protein is a Cas 12 protein and the Acr is AcrVA2 or AcrVA4. In some cases, the Cas protein is a Cas 13 protein.

In some cases, the Cas protein is provided as a split-cas protein (e.g., a Cas9 protein can in some cases be delivered as a split-Cas9, or a nucleic acid(s) encoding a split-Cas9) such that two separate proteins together form a functional Cas protein. In some such cases the sequences that encode the two parts of the split-cas protein are present on the same vector and In some cases, they are present on separate vectors, e.g., as part of a vector system that comprises the coordinated delivery system.

Also provided are methods (e.g., methods for nucleic acid targeting, cleavage, and editing). In some embodiments, the coordinated delivery system is introduced into a host cell (e.g., a eukaryotic cell such as a plant, animal, invertebrate, insect, vertebrate, mammalian, or human cell). In some embodiments, the coordinated delivery system is introduced into a host cell (e.g., a bacterial cell, an archaeal cell, or a eukaryotic cell such as a plant, animal, invertebrate, insect, vertebrate, mammalian, or human cell). The host cell can be ex vivo (e.g., fresh isolate—early passage), in vivo, or in culture in vitro (e.g., immortalized cell line). In some cases, the targeted nucleic acid (e.g., for cleavage/editing) is the host cell's genome and in some cases the targeted nucleic acid (e.g., for cleavage/editing) is from a pathogen, e.g., the genome of a pathogen within the host cell.

The on-target/off-target CRISPR complex activity referred to in a subject composition or method can include genome editing (e.g., via DNA cleavage in the presence or absence of a donor polynucleotide).

### III. BRIEF DESCRIPTION OF THE DRAWINGS

FIG. 1 depicts a schematic showing an Acr protein binding to Cas9 and inhibiting Cas9's DNA cleavage activity.

FIG. 2 depicts a schematized summary of CRISPR-Cas immunity (left) and Anti-CRISPR mechanisms (right). “Cas”: CRISPR associated gene/protein; “AcrIIA”: Anti-CRISPR for Type II-A CRISPR system, “AcrVA”: Anti-CRISPR for Type V-A CRISPR system.

FIG. 3A-3C depict schematics that illustrate CRISPR-Cas nuclease and Acr protein activities to achieve a desired targeting outcome in a cell (e.g., gene editing). (FIG. 3A) Differential rates of on-target (matched target sequence) versus off-target (mismatched sequence) editing by a Cas nuclease. Mismatches (off-target events) are expected to occur at slower rates than matches (on-target events). (FIG. 3B) control of Cas and Acr protein expression levels over time in a cell over time to achieve a desired outcome (e.g., DNA editing in this example). (FIG. 3C) schematic illustrating that limiting Cas editing with Acr inhibition reduces off-target editing while maintaining on-target editing.

FIG. 4A-4B depict an example expression vector that includes a 2A sequence (translational control element) upstream of an Acr-encoding sequence. FIG. 4A depicts a schematic drawing of a Mammalian expression vector delivering CRISPR components, single guide RNA (sgRNA), SpCas9, and Acr protein driven by U6, CMV promoter and a 2A self-cleaving peptide respectively. The numbers indicate the estimated sizes of the various payload components. FIG. 4B is a diagram showing a snapshot of the sequence joinders of the 3′ end of Cas9, nucleoplasmin NLS, 2A peptide and Acr

FIG. 5 depicts results from on-target and off-target measurements after using 2A peptides as translational control elements. All tested 2A peptides were efficient at producing Acr protein. The Acx137 was the construct that contains an Acr that does not inhibit SpCas9 and was used here as a control. The use of F2A resulted in the strongest inhibition of SpCas9 editing by Acx-105; followed by the combination E2A-F2A and T2A. The least efficient configuration was the tandem use of T2A-E2A-F2A. Also see Table 9b.

FIG. 6 depicts results from on-target and off-target measurements after using 2A peptides with Acx-153 and Acx-164. Also see Table 10.

FIG. 7A-7D depicts results from an example evaluation of a 2A peptide elements. Acx105 reduced all editing (on and off target), whereas Acx153 and Acx162 in combination with the F2A peptide had a greater effect on off-targeting, with only a moderate reduction in on-targeting editing efficiency (FIG. 7A). In comparison to the no Acr control, both Acx153 and Acx162 in combination with the F2A peptide improved the on-target to off-target ratio (FIG. 7B). The combination of Acx162 with the F2A peptide significantly reduced the off-target editing but had only a small impact on the on-target efficiency (FIG. 7C and FIG. 7D).

FIG. 8 depicts schematics of non-limiting examples of arrangements of components in a subject nucleic acid. “P1” and “P2” are promoters, “Cas” is the Cas-encoding sequence, “Acr” is the Acr-encoding sequence, “2A” is a 2A peptide encoding sequence, and “X” is a ‘spacer’ sequence.

FIG. 9 depicts a schematic of an example mammalian expression vector for delivering CRISPR components, single guide RNA (sgRNA), SpCas9, and Acr protein driven by U6, and CMV promoter. An IRES sequence is the translational control element. The numbers indicate the estimated sizes of the various payload components and the black stripe on IRES indicates where the mutations are in the variants v5, v10, v15, v21.

FIG. 10 depicts results from on-target and off-target measurements after using different IRES sequences as translational control elements. All variants V5, V10, V15 and V21 are a result of mutations on the 10th, 11th and 12th AUG segments of the IRES element. The use of the wild-type EMCV IRES element provided AcrIIA4 with a strong translation profile, allowing the Acr protein to inhibit SpCas9 activity almost completely. Variants V5 and V10 increasingly weakened translation/expression and an increase in SpCas9 editing capabilities was observed due to less Acr protein being produced. Variants V15 and V21 were responsible for very weak translation/expression and the values of SpCas9 editing were similar to those of no-Acr protein control. Also see Table 9a.

FIG. 11A-11B depicts results from evaluating IRES elements in combination with SpyCas9 and Acr variants. Background measurement (sample that did not contain either the Cas nuclease or the Acr) was subtracted and the resulting on and off target measurements are graphed in FIG. 11A. FIG. 11B shows the on/off target ratio.

FIG. 12 depicts schematics of non-limiting examples of arrangements of components in a subject nucleic acid. “P1” and “P2” are promoters, “Cas” is the Cas-encoding sequence, “Acr” is the Acr-encoding sequence, “IRES” is an IRES sequence, and “X” is a ‘spacer’ sequence.

FIG. 13A-13D depicts sequences of example IRES sequences. Top to bottom: SEQ ID NOs: 139-159.

FIG. 14 depicts an example mammalian expression vector delivering CRISPR components, single guide RNA (sgRNA), SpCas9, and Acr protein driven by U6, CMV promoter and EF1-alpha promoters respectively. AUG mutation is shown in grey at the start position of the Acr coding sequence. The numbers indicate the estimated sizes of the various payload components.

FIG. 15A-B depicts results from on-target and off-target measurements after using non-AUG start codons. The presence of a canonical start codon, AUG, resulted in strong inhibition by AcrIIA4, reducing SpCas9 editing more than 80%. The use of non-canonical start codons decreased the inhibitory profile and the mutants had a sliding effect, with CUG being the strongest with—36% inhibition of ON target editing and 50% inhibition of OFF target editing. GUG, UUG and ACG all had very weak inhibitory profiles, with editing percentages similar to no Acr control. Numbers for indel frequencies are shown in Table 9c. FIG. 15A shows on/off target efficiencies. FIG. 15B shows the on-target efficiencies with the background (“NT”) subtracted.

FIG. 16A-16B depicts results from evaluating non-canonical start sites. Acx137 and Acx105 were compared for on-target and off-target editing efficiencies. FIG. 16A shows on target and off target measurements, and FIG. 16B shows the measured on-target to off-target ratios.

FIG. 17A-17B depicts results from comparing Acx137 and Acx105 for on-target and off-target editing efficiencies. FIG. 17A shows on target and off target measurements, and FIG. 17B shows the measured on-target to off-target ratios. All three tested constructs had similar levels of on-target editing efficiency. Selectivity for on-targeting versus off-targeting editing was enhanced with the Acx162 constructs.

FIG. 18 depicts schematics two non-limiting examples of arrangements of components in a subject nucleic acid. “P1” and “P2” are promoters, “Cas” is the Cas-encoding sequence, “Acr” is the Acr-encoding sequence, Asterisks denotes a non-AUG start codon.

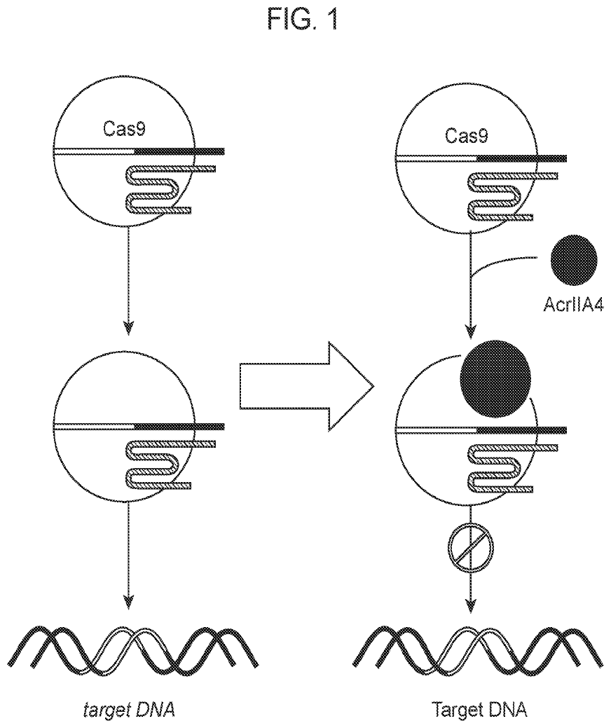

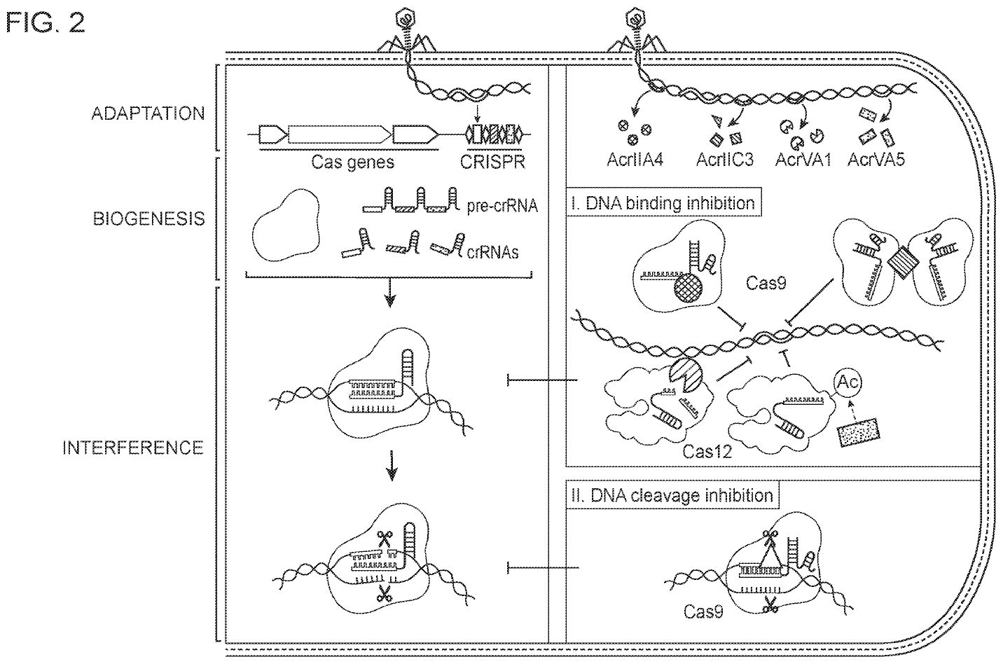

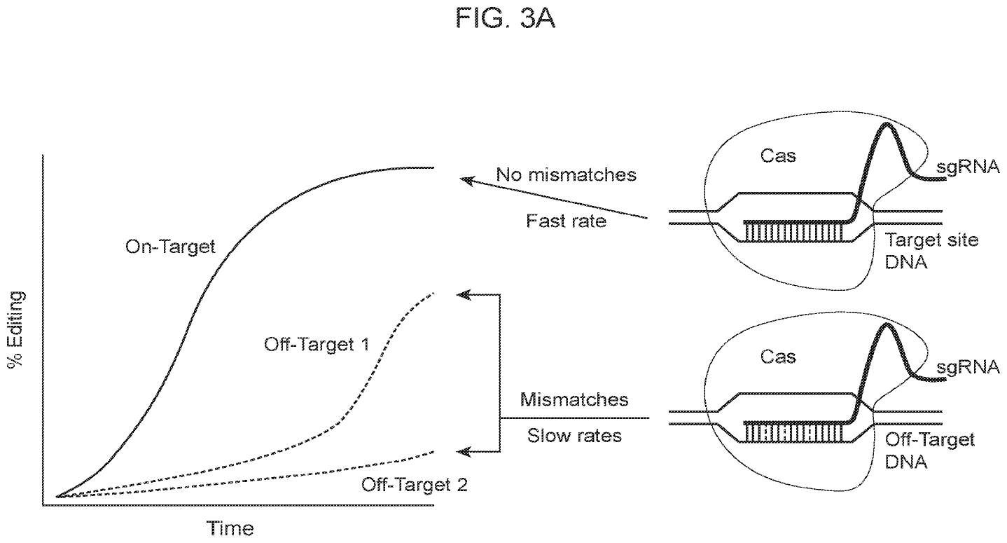

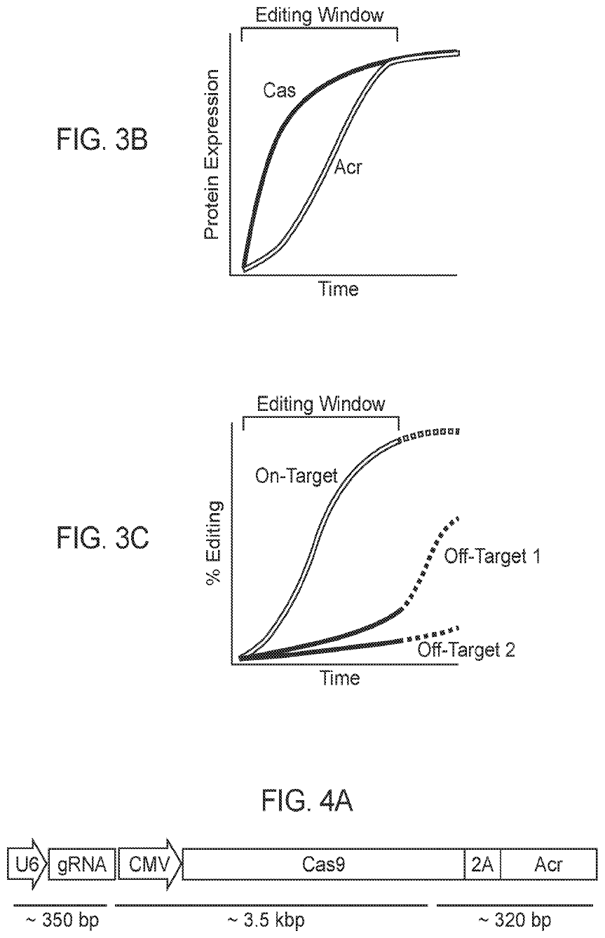

### IV. DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable”, “hybridizes”, “complementary”, or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 17 nucleotides or more, 18 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to a nucleic acid binding domain of a polypeptide, binding to a target nucleic acid, and the like) refers to a non-covalent interaction between macromolecules (e.g., between a protein and a nucleic acid such as DNA or RNA). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions can generally be characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Eukaryotic promoters will often, but not always, contain “TATA” boxes and “CAT” boxes. Various promoters, including constitutive, tissue-specific, and inducible promoters may be used to drive expression by the various vectors of the present disclosure. The level of expression of a given promoter can be described as weak, medium, or strong—and thus, promoters can be categorized as weak, medium, or strong promoters.

“Operably linked” refers to a juxtaposition wherein the components so described are in a relationship permitting them to function in their intended manner. For instance, a promoter is operably linked to a nucleotide sequence (the nucleotide sequence can also be said to be operably linked to the promoter) if the promoter affects transcription of said nucleotide sequence. As another example, a translational control element is operably linked to a protein-coding sequence (the protein-coding sequence can also be said to be operably linked to the translational control element) if the translational control element affects translation of protein from the protein-coding sequence.

A “coordinated delivery system” as used herein refers to the coordinated delivery of a an Acr protein and a Cas nuclease. A coordinated delivery system includes 1 or more nucleic acids (e.g., vectors) for expression of an Acr protein and a Cas effector protein, e.g., in a host cell. In some cases, a coordinated delivery system provides a single nucleic acid (e.g., vector) for expression of an Acr protein and a Cas effector protein. In some cases, a coordinated delivery system provides more than one nucleic acid (e.g., vector) for expression of an Acr protein and a Cas nuclease and the expression and/or function is coordinated such as with the provision of a split-Cas from 2 vectors. In some cases of the coordinated delivery system the expression and/or function of an Acr protein and a Cas nuclease is linked (i.e., coordinated) by virtue of translational control element selected to regulate the translation of the Acr protein and/or Cas effector protein.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “subject,” and “host,”,” used interchangeably herein, refer to an individual organism that expresses or is intended to express the coordinated delivery system and/or the Cas nuclease and/or Acr proteins described herein. Hosts include, but are not limited to fungi (such as yeasts), plants, algae, insects, animals such as birds and mammals, (e.g., a mammal, including, but not limited to, murines, simians, humans, mammalian farm animals, mammalian sport animals, and mammalian pets). Hosts include, but are not limited to, microbes such as bacteria and fungi (such as yeasts), plants, algae, insects, animals such as birds and mammals, (e.g., a mammal, including, but not limited to, murines, simians, humans, mammalian farm animals, mammalian sport animals, and mammalian pets).

The terms “on-target” and “off-target” are used herein to refer to the locations of CRISPR complex activity (e.g., target DNA cleavage, DNA editing) within a target DNA. Both location types (on- and off-target) are based on the guide sequence of the guide RNA. CRISPR complex mediated events that take place at a location based on a 100% match with the guide sequence are considered “on-target” while those that take place at (undesired) locations that are not based on a 100% match with the guide sequence are “off-target”. If the sequence of the target DNA is known (e.g., a large portion of the genome of a target cell has been sequenced), then likely off-target sites can be predicted for a given guide sequence. In general, off-target events are more likely to take place at sequences that are closer to a 100% match than sequences that are farther. As such, most off-target events tend to take place at sequences with 50% or more (e.g., 75% or more) sequence identity with the intended target sequence. As such, a sequence analysis of the target DNA can provide a list of expected possible off-target sites within a target DNA. The number of predicted off-target sites will depend on the target DNA sequence, but in some cases the number of predicted off-target sites will be in a range of from 10-200 predicted sites (e.g., from 10-150, 10-100, 10-50, 15-200, 15-150, 15-100, 15-80, 20-200, 20-150, 20-100, or 20-80 predicted sites).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that as used herein and in the appended claims, the singular forms “a,” “an,” and “the” include plural referents unless the context clearly dictates otherwise. Thus, for example, reference to “a protein” includes a plurality of such proteins and reference to “the protein” includes reference to one or more such proteins and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. For example, as will be apparent to those of skill in the art upon reading this disclosure, each of the individual embodiments described and illustrated herein has discrete components and features which may be readily separated from or combined with the features of any of the other several embodiments without departing from the scope or spirit of the present invention. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### V. DETAILED DESCRIPTION

As noted above, the present disclosure provides compositions and methods that provide a coordinated delivery system, where the system utilizes an Acr protein in combination with a CRISPR Cas protein to achieve a balance in which Cas protein retains sufficient activity to perform the desired on-target nucleic acid functions (e.g., DNA cleavage for gene editing applications), but is inhibited by one or more Acr proteins to a degree that decreases off-target activity. The Acr protein is an inhibitor of the Cas protein, and the translational control element can provide for an expression ratio of the Acr protein to the Cas protein in a host cell sufficient to increase the ratio of on-target to off-target nucleic acid activity (e.g., cleavage) of a CRISPR complex, e.g., relative to the ratio of on-target to off-target nucleic acid activity of said CRISPR complex in the absence of the Acr protein. The coordinated delivery system retains a sufficient level of on-target nucleic acid activity such that the desired or intended outcome (e.g., nucleic acid editing) is accomplished and the CRISPR complex activity is not completely inhibited (i.e., at least some detectable activity is retained). In some embodiments, the coordinated delivery system includes more than one vector such as that include sequences encoding a split-cas protein such as split-Cas9 and methods (e.g., methods for nucleic acid targeting). In some embodiments a subject vector or vector system is introduced into a host (e.g. organism) or host cell (e.g., a eukaryotic cell such as a plant, animal, invertebrate, insect, vertebrate, mammalian, or human cell). In some embodiments a subject vector or vector system is introduced into a host (e.g. organism) or host cell (e.g., a bacterial cell, an archaeal cell, or a eukaryotic cell such as a plant, animal, invertebrate, insect, vertebrate, mammalian, or human cell).

### CRISPR Complex

The terms “CRISPR complex” and “effector complex” as used herein refer to the protein-RNA complex that is guided to a specific sequence within a target nucleic acid (e.g., target genomic DNA) by the RNA component—often referred to as a “guide RNA” (the term “guide RNA” is discussed in more detail below). In Class 2 CRISPR systems, the functions of the effector complex are carried out by a single protein (which can be referred to as an “effector protein”)—where the natural protein is an endonuclease (e.g., see Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; and Shmakov et al., Nat Rev Microbiol. 2017 March; 15(3):169-182: “Diversity and evolution of class 2 CRISPR-Cas systems”).

As such, the term “class 2 CRISPR/Cas protein” or “CRISPR/Cas effector protein” or more simply “Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

In Class 1 CRISPR systems (e.g., type I, III, and IV systems), the functions of the effector complex (CRISPR complex) are carried out by multiple proteins. Examples include the ‘Cascade’ of type I systems and the Csm-Cmr complexes of type III systems.

### Acr and Cas Proteins

Acr proteins of the disclosure are proteins that inhibit Cas proteins—thereby acting as negative regulators of the CRISPR complex. In some cases, a subject Acr protein is an inhibitor of a Cas protein of a class 2 CRISPR complex (e.g., a class 2 effector protein such as Cas9 or a Cas12 protein such as Cas12a—also known as Cpf1)—thereby directly regulating the effector protein of a CRISPR complex. In some cases, a subject Acr protein is an inhibitor of a Cas protein of a class 1 CRISPR complex. In such cases, the Acr protein can be an inhibitor of any of the proteins of the complex as long as the inhibition negatively regulates the overall activity/function of the complex.

The effector CRISPR-Cas nucleases that complex with gRNA are highly diverse and spread across 6 distinct types (Types I-VI). So far, anti-CRISPR proteins (Acrs) have been discovered that inhibit CRISPR Type I, II, and V systems. The CRISPR-Cas Type II-A orthologue from S. pyogenes, SpCas9, is the most widely utilized CRISPR-Cas enzyme for biotechnological applications and has also been deployed in DNA-binding applications. Acr proteins that function against the Type II-A system have been discovered by a bioinformatics approach that surveys bacterial genomes for self-targeting. Bioinformatic identification of self-targeting Type II-A CRISPR systems followed by discovery of neighboring genes (the “guilt by association” strategy) has recently led to discovery of SaCas9 inhibitors.

The conservation of Acr-associated genes has served as a signpost for identifying novel Acr genes. Among the 44 distinct families of Acr proteins discovered so far, specific inhibitory mechanisms have been determined for 11 of them (AcrIE1, AcrIF1-3, AcrIF10, AcrIIA2, AcrIIA4, AcrIIC1-3, and AcrVA5). The known mechanisms are highly diverse, presenting a natural pool of off-switch modalities to draw from. Acr proteins can act at 3 different steps of CRISPR-Cas-mediated immunity including 1) inhibiting guide RNA loading, 2) blocking DNA binding, and 3) preventing DNA cleavage. The most common mechanism observed to date is that the anti-CRISPR protein occupies the DNA-binding site on the Cas protein, thus mimicking DNA and inhibiting the DNA-binding and cleavage activity of the protein.

However, the mechanisms by which Acrs block DNA binding can be different. For example, even though AcrIF1, AcrIF2, and AcrIF10 bind to different subunits of the cascade effector complex of the type I-F CRISPR-Cas system, they all prevent DNA binding to the complex. AcrIIC3 also blocks DNA binding but uses a fourth and distinct mechanism—promoting dimerization of Cas9. For the most potent SpCas9 inhibitor, AcrIIA4, a 3.9-Å resolution cryo-electron structure revealed that the Cas9-sgRNA-AcrIIA4 complex has AcrIIA4 bound to the PAM-interacting domain of Cas9, thus preventing the target DNA binding. Interestingly, AcrIIA4 binds only to assembled Cas9-sgRNA complexes, not to Cas9 protein alone or to preformed Cas9-sgRNA-DNA complexes. More recent biochemical studies have shown that each newly discovered anti-SaCas9 protein (AcrIIA13-15) mediates DNA cleavage by a distinct mechanism. More specifically, AcrIIA13 and AcrIIA15 inhibit dsDNA binding of Cas9, but only when added before the addition of target dsDNA, while AcrIIA14 completely inhibited Cas9 from binding to its target no matter when it was added to the reaction.

It is to be understood that when discussing particular proteins such as Cas or

Acr proteins throughout this disclosure (e.g., “Cas9”, “Cas12a”, “AcrIIA1”), and when presenting such terms in claims, such terms are intended to encompass modified/mutated versions of such proteins that maintain their intended function. As an illustrative example, in some cases an effector protein (e.g., Cas9), is mutated such that it has nickase activity (cleaves only one strand of a double stranded target). Thus, for example, the terms are intended to encompass embodiments in which an effector protein has reduced nuclease activity (e.g., has nickase activity). Likewise, the terms are intended to encompass embodiments in which is the Cas and/or Acr protein is fused to one or more heterologous proteins (e.g., a fluorescent protein such as GFP, one or more nuclear localization signals (NLSs), and/or a tag such as MBP, CBP, strep tag, GST, HA, poly(His), Myc, V5, Spot, NE, AviTag, and the like).

Thus, in some embodiments a subject “Acr protein” comprises the wild type (natural) sequence. In some cases, a subject Acr Protein is mutated. As one non-limiting example, the Acr protein can be an AcrIIA2 with an amino acid replacement at one or more positions, for example one or more selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122 and K123. As another non-limiting example, the Acr protein can be an AcrIIA4 with an amino acid replacement at one or more positions, for example one or more selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87. In some of the above cases, the one or more positions are replaced with an alanine or with an arginine. In some cases, the one or more positions are replaced with a conservative amino acid change, such as one that preserves charge or size or shape of the amino acid. In some cases, the one or more positions are replaced with a non-conservative amino acid change, such as one that alters charge, size and/or shape of the amino acid. In some cases, the Acr protein is AcrIIA4, and in some such cases the AcrIIA4 comprises one or more amino acid mutations (replacements) selected from: D14A, G38A, N39A, and any combination thereof (e.g., in some cases N39A or the amino acid replacements D14A and G38A).

In some cases (e.g., see the preceding paragraph), the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with a wild type Acr protein (see, e.g., the Acr sequences of Table 2—SEQ ID NOs: 1-79). In some cases, the Acr protein comprises an amino acid sequence having 85% or more sequence identity (e.g., 90% or more, 95% or more, 97% or more, 98% or more, 99% or more, or 100% sequence identity) with a wild type Acr protein (see, e.g., the Acr sequences of Table 2—SEQ ID NOs: 1-79). In some cases, the Acr protein comprises an amino acid sequence having 90% or more sequence identity (e.g., 95% or more, 97% or more, 98% or more, 99% or more, or 100% sequence identity) with a wild type Acr protein (see, e.g., the Acr sequences of Table 2—SEQ ID NOs: 1-79). In some cases, the Acr protein comprises an amino acid sequence having 95% or more sequence identity (e.g., 97% or more, 98% or more, 99% or more, or 100% sequence identity) with a wild type Acr protein (see, e.g., the Acr sequences of Table 2—SEQ ID NOs: 1-79). In some cases, the Acr protein comprises the amino acid sequence of a wild type Acr protein (see, e.g., the Acr sequences of Table 2—SEQ ID NOs: 1-79).

In some cases, the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with an Acr protein sequence set forth in any one of SEQ ID NOs: 1-82 and 161. In some cases, the Acr protein comprises an amino acid sequence having 85% or more sequence identity (e.g., 90% or more, 95% or more, 97% or more, 98% or more, 99% or more, or 100% sequence identity) with an Acr protein sequence set forth in any one of SEQ ID NOs: 1-82 and 161. In some cases, the Acr protein comprises an amino acid sequence having 90% or more sequence identity (e.g., 95% or more, 97% or more, 98% or more, 99% or more, or 100% sequence identity) with an Acr protein sequence set forth in any one of SEQ ID NOs: 1-82 and 161. In some cases, the Acr protein comprises an amino acid sequence having 95% or more sequence identity (e.g., 97% or more, 98% or more, 99% or more, or 100% sequence identity) with an Acr protein sequence set forth in any one of SEQ ID NOs: 1-82 and 161. In some cases, the Acr protein comprises the amino acid sequence of an Acr protein sequence set forth in any one of SEQ ID NOs: 1-82 and 161.

In some cases, the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with an Acr protein sequence set forth in any one of SEQ ID NOs: 80-82. In some cases, the Acr protein comprises an amino acid sequence having 85% or more sequence identity (e.g., 90% or more, 95% or more, 97% or more, 98% or more, 99% or more, or 100% sequence identity) with an Acr protein sequence set forth in any one of SEQ ID NOs: 80-82. In some cases, the Acr protein comprises an amino acid sequence having 90% or more sequence identity (e.g., 95% or more, 97% or more, 98% or more, 99% or more, or 100% sequence identity) with an Acr protein sequence set forth in any one of SEQ ID NOs: 80-82. In some cases, the Acr protein comprises an amino acid sequence having 95% or more sequence identity (e.g., 97% or more, 98% or more, 99% or more, or 100% sequence identity) with an Acr protein sequence set forth in any one of SEQ ID NOs: 80-82. In some cases, the Acr protein comprises the amino acid sequence of an Acr protein sequence set forth in any one of SEQ ID NOs: 80-82.

In some cases, the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with an Acr protein sequence set forth in any one of SEQ ID NOs: 81-82 and 161. In some cases, the Acr protein comprises an amino acid sequence having 85% or more sequence identity (e.g., 90% or more, 95% or more, 97% or more, 98% or more, 99% or more, or 100% sequence identity) with an Acr protein sequence set forth in any one of SEQ ID NOs: 81-82 and 161. In some cases, the Acr protein comprises an amino acid sequence having 90% or more sequence identity (e.g., 95% or more, 97% or more, 98% or more, 99% or more, or 100% sequence identity) with an Acr protein sequence set forth in any one of SEQ ID NOs: 81-82 and 161. In some cases, the Acr protein comprises an amino acid sequence having 95% or more sequence identity (e.g., 97% or more, 98% or more, 99% or more, or 100% sequence identity) with an Acr protein sequence set forth in any one of SEQ ID NOs: 81-82 and 161. In some cases, the Acr protein comprises the amino acid sequence of an Acr protein sequence set forth in any one of SEQ ID NOs: 81-82 and 161.

Likewise, in some cases, a subject “Cas protein” comprises the wild type (natural) sequence. In some cases, the Cas protein comprises an amino acid sequence having 70% or more sequence identity (e.g., 75% or more, 80% or more, 85% or more, 90% or more, 95% or more, 97% or more, 98% or more, 99% or more, or 100% sequence identity) with a wild type Cas protein (see, e.g., see the Cas protein sequences of Table 3). In some cases, the Cas protein comprises an amino acid sequence having 80% or more sequence identity (e.g., 85% or more, 90% or more, 95% or more, 97% or more, 98% or more, 99% or more, or 100% sequence identity) with a wild type Cas protein (see, e.g., see the Cas protein sequences of Table 3). In some cases, the Cas protein comprises an amino acid sequence having 90% or more sequence identity (e.g., 95% or more, 97% or more, 98% or more, 99% or more, or 100% sequence identity) with a wild type Cas protein (see, e.g., see the Cas protein sequences of Table 3). In some cases, the Cas protein comprises an amino acid sequence having 95% or more sequence identity (e.g., 97% or more, 98% or more, 99% or more, or 100% sequence identity) with a wild type Cas protein (see, e.g., see the Cas protein sequences of Table 3). In some cases, the Cas protein comprises the amino acid sequence of a wild type Cas protein (see, e.g., see the Cas protein sequences of Table 3). In some cases, a subject Cas protein has been ‘evolved’ such that it has low overall sequence homology to the natural Cas protein, but retains the identifiable characteristic domain(s) of that protein. In some cases, a subject Cas protein is a Cas3 protein. In some cases, a subject Cas protein is a Cas9 protein. In some cases, a subject Cas protein is a Cas12a protein.

Suitable class 2 effector proteins that can be used as a subject Cas protein include but are not limited to: Cas9 (e.g., SpCas9, NmeCas9, saCas9), Cas12 (e.g., Cas12a also known as Cpf1, Cas12b also known as C2c1, Cas12c also known as C2c3, Cas12d also known as CasY, Cas12e also known as CasX, and the like), and Cas 13 (e.g., Cas13a, Cas13b, Cas13d, and the like). In some cases, a subject cas protein is not an effector protein of a class 2 CRISPR system. For example, a cas protein can be one of the proteins that make up the CRISPR complex (‘Cascade’ or ‘Csm-Cmr’) of a class 1 CRISPR system (e.g., type I or type III CRISPR system, respectively).

In some cases, a subject cas protein (e.g., when the cas protein is a class 2 effector protein) has been mutated such that it has reduced catalytic activity. In some such cases this can render the protein to be a nickase (cleaves one strand of a double stranded target but not the other strand). Catalytic residues of class 2 effector proteins are readily identifiable and are readily found in the literature. For example, mutations that affect the RuvC or HNH domains of Cas9 (such as D10A or H840A of SpCas9, respectively), result in a nickase, while mutations that inactivate both domains (e.g., the double mutant D10A, H840A) result in a ‘dead’ Cas9 protein (dCas9).

In some cases, a protein (e.g., a Cas protein such as Cas9 or Cas12a, an Acr protein) is fused to one or more heterologous polypeptides (also referred to herein as fusion partners) (e.g., one or more NLSs, a protein tag, and the like). Suitable fusion partners include but are not limited to: (i) subcellular localization sequences (e.g., one or more, two or more, or three or more nuclear localization signals (NLSs) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like); (ii) protein tags, e.g., for ease of tracking and/or purification (e.g., a fluorescent protein, such as green fluorescent protein (GFP), YFP, RFP, CFP, mCherry, tdTomato, and the like, MBP, CBP, strep tag, GST, HA, FLAG, poly(His), Myc, V5, Spot, NE, AviTag, and the like); and (iii) polypeptides that provide for increased or decreased stability (e.g., a degron, which in some cases is controllable, e.g., a temperature sensitive or drug controllable degron sequence).

A subject protein (e.g., a Cas protein such as Cas9 or Cas12a, an Acr protein) can have multiple (1 or more, 2 or more, 3 or more, etc.) fusion partners in any combination. As an illustrative example, a subject protein can have a fusion partner that provides for tagging (e.g., GFP), and can also have a subcellular localization sequence (e.g., one or more NLSs). In some cases, such a fusion protein might also have a tag for ease of tracking and/or purification. As another illustrative example, a subject protein can have one or more NLSs (e.g., two or more, three or more, four or more, five or more, 1, 2, 3, 4, or 5 NLSs). In some cases, a fusion partner is located at or near the C-terminus (e.g., within about 50 amino acids of the C-terminus), near the N-terminus (e.g., within about 50 amino acids of the N-terminus), or at both the N-terminus and C-terminus. In some cases, a Cas protein (e.g., Cas9) that is fused to a heterologous polypeptide is also mutated (relative to the natural Cas protein sequence) such that it has nickase activity.

Non-limiting examples of NLSs include an NLS sequence derived from: the NLS of the SV40 virus large T-antigen, having the amino acid sequence PKKKRKV (SEQ ID NO: 87); the NLS from nucleoplasmin (e.g., the nucleoplasmin bipartite NLS with the sequence KRPAATKKAGQAKKKK (SEQ ID NO: 88)); the c-myc NLS having the amino acid sequence PAAKRVKLD (SEQ ID NO: 89) or RQRRNELKRSP (SEQ ID NO: 90); the hRNPA1 M9 NLS having the sequence NQSSNFGPMKGGNFGGRSSGPYGGGGQYFAKPRNQGGY (SEQ ID NO: 91); the sequence RMRIZFKNKGKDTAELRRRRVEVSVELRKAKKDEQILKRRNV (SEQ ID NO: 92) of the IBB domain from importin-alpha; the sequences VSRKRPRP (SEQ ID NO: 93) and PPKKARED (SEQ ID NO: 94 of the myoma T protein; the sequence PQPKKKPL (SEQ ID NO: 95) of human p53; the sequence SALIKKKKKMAP (SEQ ID NO: 96) of mouse c-abl IV; the sequences DRLRR (SEQ ID NO: 97) and PKQKKRK (SEQ ID NO: 98) of the influenza virus NS1; the sequence RKLKKKIKKL (SEQ ID NO: 99) of the Hepatitis virus delta antigen; the sequence REKKKFLKRR (SEQ ID NO: 100) of the mouse Mx1 protein; the sequence KRKGDEVDGVDEVAKKKSKK (SEQ ID NO: 101) of the human poly(ADP-ribose) polymerase; and the sequence RKCLQAGMNLEARKTKK (SEQ ID NO: 102) of the steroid hormone receptors (human) glucocorticoid. In general, NLS (or multiple NLSs) are of sufficient strength to drive accumulation of the Cas protein in a detectable amount in the nucleus of a eukaryotic cell. In some cases, a fusion partner includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of a polypeptide and in some embodiments, a PTD is covalently linked to the carboxyl terminus of a polypeptide. In some cases, the PTD is inserted internally at a suitable insertion site. In some cases, a subject Cas protein includes (is conjugated to, is fused to) one or more PTDs (e.g., two or more, three or more, four or more PTDs). Examples of PTDs include but are not limited to a minimal undecapeptide protein transduction domain (corresponding to residues 47-57 of HIV-1 TAT comprising YGRKKRRQRRR; SEQ ID NO:103); a polyarginine sequence comprising a number of arginines sufficient to direct entry into a cell (e.g., 3, 4, 5, 6, 7, 8, 9, 10, or 10⁻⁵⁰ arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:104); Transportan GWTLNSAGYLLGKINLKALAALAKKIL (SEQ ID NO:105); KALAWEAKLAKALAKALAKHLAKALAKALKCEA (SEQ ID NO:106); and RQIKIWFQNRRMKWKK (SEQ ID NO:107). Exemplary PTDs include but are not limited to, YGRKKRRQRRR (SEQ ID NO:108), RKKRRQRRR (SEQ ID NO:109); an arginine homopolymer of from 3 arginine residues to 50 arginine residues; Exemplary PTD domain amino acid sequences include, but are not limited to, any of the following: YGRKKRRQRRR (SEQ ID NO:110); RKKRRQRR (SEQ ID NO:111); YARAAARQARA (SEQ ID NO:112); THRLPRRRRRR (SEQ ID NO:113); and GGRRARRRRRR (SEQ ID NO:114). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some cases, a Cas effector protein is fused to a heterologous protein sequence that provides for an activity (e.g., nuclease activity such as that provide by FokI nuclease, protein modification activity such as histone modification activity including acetylation or deacetylation or demethylation or methyltransferase activity, transcription modulation activity such as activity provided by fusing the Cas protein to a transcriptional activator or repressor, base editing activity such as deaminase activity, DNA modifying activity such as DNA methylation activity, and the like). In some such cases the Cas effector protein has nickase activity or is catalytically inactive, e.g., the Cas effector protein can be mutated such that it now longer has cleavage activity and instead the activity is provided by the heterologous polypeptide to which the Cas protein is fused.

### Linkers (e.g., for Fusion Partners)

In some embodiments, a subject Cas protein can fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)n, glycine-serine polymers (including, for example, (GS)n, GSGGSn (SEQ ID NO: 115), GGSGGSn (SEQ ID NO: 116), and GGGSn (SEQ ID NO: 117 where n is an integer of at least one), glycine-alanine polymers, alanine-serine polymers. Exemplary linkers can comprise amino acid sequences including, but not limited to, GGSG (SEQ ID NO: 118), GGSGG (SEQ ID NO: 119), GSGSG (SEQ ID NO: 120), GSGGG (SEQ ID NO: 121), GGGSG (SEQ ID NO: 122), GSSSG (SEQ ID NO: 123), and the like. The ordinarily skilled artisan will recognize that design of a peptide conjugated to any desired element can include linkers that are all or partially flexible, such that the linker can include a flexible linker as well as one or more portions that confer less flexible structure.

Suitable Acr proteins include those that inhibit any desired Cas proteins. Table 1 and Table 2 include examples of Acr proteins. Examples of suitable Acr proteins include, but are not limited to, those that inhibit class 1 Cas proteins such as Cas proteins from type I, type III, or type IV CRISPR systems; as well as those that inhibit class 2 Cas effector proteins such as type II proteins (e.g., Cas9), type V proteins (e.g., Cpf1/Cas12a, C2c1/Cas12b, C2C3/Cas12c) and type VI proteins (e.g., C2c2/Cas13a, C2c6/Cas13b, C2C7/Cas13c).

TABLE 1
Examples of Acr proteins and their target Cas proteins

| | | CRISPR | |
|---|---|---|---|
| Acr | | system | |
| protein | Origin | inhibited | Mechanism Of Action |
| AcrIIA1 | Listeria (L) monocytogenes prophage | II-A (Lmo) | Binds Cas9, inhibits cleavage, triggers |
| | J0161a | | degradation |
| AcrIIA2 | L monocytogenes prophage J0161a | II-A (Lmo, Spy) | Binds Cas9, in PAM interaction motif |
| AcrIIA3 | L monocytogenes prophage SLCC2482 | II-A (Lmo) | unknown |
| AcrIIA4 | L monocytogenes prophage J0161b | II-A (Lmo, Spy) | Binds Cas9, in PAM interaction motif |
| AcrIIA5 | Streptococcus (S) thermophilus phage | II-A (Sth, Spy, Sau) | Destabilizes Cas9, sgRNA gets cleavaed |
| | D4276 | | somehow |
| AcrIIA6 | S thermophilus phage D1811 | II-A (Sth) | Dimerizes Cas9, allosterically prevents |
| | | | DNA binding |
| AcrIIA7 | Metagenomic libraries from human gut | II-A (Spy) | unknown |
| AcrIIA8 | Metagenomic libraries from human gut | II-A (Spy) | unknown |
| AcrIIA9 | Metagenomic libraries from human gut | II-A (Spy) | unknown |
| AcrIIA10 | Metagenomic libraries from human gut | II-A (Spy) | unknown |
| AcrIIA11 | Clostridium sp. from human gut | II-A (Spy) | Vague (Forsberg et al Elife) |
| | metagenome | | |
| AcrIIA12 | L monocytogenes prophage | II-A (Sa, Lmo) | Likely Binds Cas9, PAM interaction domain |
| AcrIIA13 | Staphylococcus schleiferi strain | II-A (Sa) | Blocks DNA binding |
| AcrIIA14 | Staphylococcus simulans strain | II-A (Sa) | Might bind SaCas9 active site |
| | | | without inhibiting DNA binding |
| | | | or triggering complex dimerization |
| | | | to form an inactive conformation |
| AcrIIA15 | unclear (Watters et al) | II-A (Sa) | Blocks DNA binding-Binds directly |
| | | | to SaCas9 to prevent RNP formation |
| AcrIIA16 | Listeria monocytogenes plasmid | II-A (Sp, Sa, St1, Nme) | Potential sgRNA cleavage |
| AcrIIA16 | Enterococcus faecalis | II-A | |
| AcrIIA17 | Enterococcus faecalis plasmid | II-A (Sa, Nme) | Potential sgRNA cleavage or loading |
| | | | interference |
| AcrIIA17 | Streptococcus gallolyticus | II-A | |
| AcrIIA18 | Streptococcus gallolyticus prophage | II-A (Spy) | Unknown |
| AcrIIA18 | Streptococcus macedonicus | II-A | |
| AcrIIA19 | Staphylococcus simulans MGE | II-A (Spy) | Potential sgRNA cleavage or loading |
| | | | interference |
| AcrIIC1 | Neisseria meningitidis | II-C (Nme, Cje, Geo, | Binds HNH domain, prevents cleavage |
| | | Hpa, Smu) | |
| AcrIIC2 | N meningitidis prophage | II-C (Nme, Hpa, Smu) | Prevents sgRNA loading |
| AcrIIC3 | N meningitidis prophage | II-C (Nme, Hpa, Smu) | Dimerizes Cas9, prevents DNA binding |
| AcrIIC4 | Haemophilus parainfluenzae prophage | II-C (Nme, Hpa, Smu) | Blocks DNA binding |
| AcrIIC5 | Simonsiella muelleri prophage | II-C (Nme, Hpa, Smu) | Blocks DNA binding |
| AcrVA1 | M bovoculi prophage | V-A (Mb, As, Lb, Fn) | crRNA cleavage |
| AcrVA2 | M bovoculi prophage | V-A (Mb) | potentially cleaves mRNA of Cas12 |
| AcrVA3 | M bovoculi prophage | V-A (Mb) | unknown |
| AcrVA4 | M bovoculi mobile element | V-A (Mb, Lb) | Induces Cas12 dimer, allosteric |
| | | | inhibition of DNA binding |
| AcrVA5 | M bovoculi mobile element | V-A (Mb, Lb) | Acetyltrasnferase that puts PTM |
| | | | on PAM interacting residue. |

| Acr | | CRISPR-Cas |
|---|---|---|
| Protein | Origin | system inhibited |
| AcrIC1 | Moraxella (M) bovoculi prophage | I-C (Pae) |
| AcrID1 | Sulfolobus islandicus rudivirus 3 | I-D (Sis) |
| AcrIE1 | Pseudomonas (P) aeruginosa phage JBD5 | I-E (Pae) |
| AcrIE2 | P aeruginosa phage JBD88a | I-E (Pae) |
| AcrIE3 | P aeruginosa phage DMS3 | I-E (Pae) |
| AcrIE4 | P aeruginosa phage D3112 | I-E (Pae) |
| AcrIE4-IF7 | Ps citronellolis prophage | I-E/I-F (Pae) |
| AcrIE5 | P otitidis prophage | I-E (Pae) |
| AcrIE6 | P aeruginosa prophage | I-E (Pae) |
| AcrIE7 | P aeruginosa prophage | I-E (Pae) |
| AcrIF1 | P aeruginosa phage JBD30 | I-F (Pae, Pec) |
| AcrIF2 | P aeruginosa phage D3112 | I-F (Pae, Pec) |
| AcrIF3 | P aeruginosa phage JBD5 | I-F (Pae) |
| AcrIF4 | P aeruginosa phage JBD26 | I-F (Pae) |
| AcrIF5 | P aeruginosa phage JBD5 | I-F (Pae) |
| AcrIF6 | P aeruginosa prophage | I-E (Pae),/I-F |
| | | (Pae, Pec) |
| AcrIF7 | P aeruginosa prophage | I-F (Pae, Pec) |
| AcrIF8 | Pectobacterium phage ZF40 | I-F (Pae, Pec) |
| AcrIF9 | Vibrio parahaemolyticus mobile element | I-F (Pae, Pec) |
| AcrIF10 | Shewanella xiamenensis prophage | I-F (Pae, Pec) |
| AcrIF11 | P aeruginosa prophage | I-F (Pae) |
| AcrIF12 | P aeruginosa mobile element | I-F (Pae) |
| AcrIF13 | M catarrhalis prophage | I-F (Pae) |
| AcrIF14 | Moraxella phage Mcat5 | I-F (Pae) |
| AcrIIIB1 | Sulfolobus islandicus | III-B |

TABLE 2
Examples of Acr proteins

| | Cas | | SEQ ID |
|---|---|---|---|
| Acr | inhibited | Amino Acid sequence | NO: |
| AcrIC1_mbo | Cas3 | MNNLKKTAITHDGVFAYKNTETVIGSVGRNDI | 1 |
| | | VMAIDATHGEFNDKNFIIYADTNGNPIYLGYA | |
| | | YLDDNNDAHIDLAVGACNEDDDFDEKEIHEMI | |
| | | AEQMELAKRYQELGDTVHGTTRLAFDDDGY | |
| | | MTVRLDQQAYPDYRPENDDKHIMWRALALT | |
| | | ATGKELEVFWLVEDYEDEEVNSWDFDIADD | |
| | | WREL | |
| AcrIC3_pae | Cas3 | MSIQVTSTNGRTVNLEIELGSVVASSGQVKF | 2 |
| | | MADKTDRGLESRFLVPEAGNRRIEVALTGRD | |
| | | LEAANALFSELAASVEATNEMYRELDAERAQI | |
| | | NKALEG | |
| AcrIC4_pae | Cas3 | MDNKITPADEEKIREWLNCEEASVDNDGDV | 3 |
| | | WWVAVPMTGHWLSDEQKAKYIEWRGDET | |
| AcrIC5_pae | Cas3 | MSKVTLNGQQIDFDAAVNLMDAELREELHSA | 4 |
| | | QEWTNDQEFLDAYVQAHAAKFDGEEFQVA | |
| AcrIC6_pae | Cas3 | MTESLIHLRVPAATKGRWWRASRAVGLRLTD | 5 |
| | | YITQAVEAYMQQQLTRVAIPDDIEFSDLKLAR | |
| | | DPDGAVSFDWAVIERICHASGLPLEMMRDAP | |
| | | EDNVASLIIGWYQAHRADGGAADPVADDLIA | |
| | | EAMAEDAAGQQFSHQPGRA | |
| AcrIC7_pae | Cas3 | MATVTKITLNGQNHYNFGSECSEADAEGYRE | 6 |
| | | WIAQELAENFPGAEIEINEADSTYSVVVEIDD | |
| | | ESYYDEARGLKDDVNVFCIDAWDRCPWDW | |
| | | VS | |
| AcrIC8_pae | Cas3 | MYAIRKIQFFYGPTDKKSYVGEEAGGRRELF | 7 |
| | | KTRAEAQARIEDLEEGVYYLAHNESGRPDYK | |
| | | IVWVRGEAQFEHARWMRG | |
| AcrID1_sis | Cas3 | MNYKELEKMLDVIFENSEIKEIDLFFDPEVEIS | 8 |
| | | KQEFEDLVKNADPLQKVVGDNYITETFEWW | |
| | | EFENQYLEFELDYYVKDEKIFVLEMHFWRKIR | |
| | | KLE | |
| AcrIE1_pae | Cas3 | MEKKLSDAQVALVAAWRKYPDLRESLEEAA | 9 |
| | | SILSLIVFQAETLSDQANELANYIRRQGLEEAE | |
| | | GACRNIDIMRAKWVEVCGEVNQHGIRVYGD | |
| | | AIDRDVD | |
| AcrIE2_pae | Cas3 | MNTYLIDPRKNNDNSGERFTVDAVDITAAAK | 10 |
| | | SAAQQILGEEFEGLVYRETGESNGSGMFQA | |
| | | YHHLHGTNRTETTVGYPFHVMEL | |
| AcrIE3_pae | Cas3 | MKITNDTTTYEVAELMGSEADELDGRIMMGL | 11 |
| | | LSRECVVDTDDLSEDQWLALIDESQKVRREQ | |
| | | FESDEA | |
| AcrIE4_pae | Cas3 | MSTQYTYEQIAEDFRLWGEYMDPNAEMTEE | 12 |
| | | EFQALSTEEKVAMQVEAFGAEA | |
| AcrIE5_pae | Cas3 | MSNDRNGIINQIIDYTGTDRDHAERIYEELRA | 13 |
| | | DDRIYFDDSVGLDRQGLLIREDVDLMAVAAEI | |
| | | E | |
| AcrIE6_pae | Cas3 | MNNDTEVLEQQIKAFELLADELKDRLPTLEIL | 14 |
| | | SPMYTAVMVTYDLIGKQLASRRAELIEILEEQ | |
| | | YPGHAADLSIKNLCP | |
| AcrIE7_pae | Cas3 | MIGSEKQVNWAKSIIEKEVEAWEAIGVDVRE | 15 |
| | | VAAFLRSISDARVIIDNRNLIHFQSSGISYSLES | |
| | | SPLNSPIFLRRFSauCSVGFEEIPTALQRIRSV | |
| | | YTAKLLEDE | |
| AcrIE4-IF7_ | Cas3 | MSTQYTYQQIAEDFRLWSEYVDTAGEMSKD | 16 |
| pae | | EFNSLSTEDKVRLQVEAFGEEKSPKFSTKVT | |
| | | TKPDFDGFQFYIEAGRDFDGDAYTEAYGVAV | |
| | | PTNIAARIQAQAAELNAGEWLLVEHEA | |
| AcrIF1_pae | Cas3 | MKFIKYLSTAHLNYMNIAVYENGSKIKARVEN | 17 |
| | | VVNGKSVGARDFDSTEQLESWFYGLPGSGL | |
| | | GRIENAMNEISRRENP | |
| AcrIF2_pae | Cas3 | MIAQQHKDTVAACEAAEAIAIAKDQVWDGEG | 18 |
| | | YTKYTFDDNSVLIQSGTTQYAMDADDADSIK | |
| | | GYADWLDDEARSAEASEIERLLESVEEE | |
| AcrIF3_pae | Cas3 | MSSTISDRIISRSVIEAARFIQSWEDADPDNLT | 19 |
| | | ESQVLAASSFAARLHEGLQATVLQRLVDESN | |
| | | RDEYREFQAWEEALLNADGRVTSNPFADWG | |
| | | WWYRIANVMLATASQNVGVAWGSHVHGRL | |
| | | MAIFQDRFQQHYEDEEC | |
| AcrIF4_pae | Cas3 | MMTISKTDIDCYLQTYVVIDPVSNGWQWGID | 20 |
| | | ENGVGGALHHGRVEMVEGENGYFGLRGAT | |
| | | HPTEKEAMAAALGYLWKCRQDLVAIARNDAI | |
| | | EAEKYRAKA | |
| AcrIF5_pae | Cas3 | MSRPTVVTVTETPRNPGSYEVNVERDGKMV | 21 |
| | | VGRARAGSDPGAAAAKAMQMAMEWGSPNY | |
| | | VILGSNKVLAFIPEQLRVKM | |
| AcrIF6_pae | Cas3 | MKVPAFFAANILTIEQIIEAINNDGSAMTSAPEI | 22 |
| | | AGYYAWDAATDALESENDLEQLTEDDFVAHL | |
| | | EVLEERGAKIDRDAAIAVALQFQAAAVNDLHS | |
| | | GDE | |
| AcrIF7_pae | Cas3 | MSHASHNGEAPKRIEAMTTFTSIVTTNPDFG | 23 |
| | | GFEFYVEAGQQFDDSAYEEAYGVSVPSAVV | |
| | | EEMNAKAAQLKDGEWLNVSHEA | |
| AcrIF8_pca | Cas3 | MARIAPNEDSTMSTAYIIFNSSVAAVVDTEIAN | 24 |
| | | GANVTFSTVTVKEEINANRDFNLVNAQNGKIS | |
| | | RAKRWGNEASKCEYFGREINPTEFFIK | |
| AcrIF9_vpa | Cas3 | MKAAYIIKEVQNINSEREGTQIEATSLSQAKRI | 25 |
| | | ASKEQCFHGTVMRIETVNGLWLAYKEDGKR | |
| | | WWDCQ | |
| AcrIF10_sxi | Cas3 | MTTFRIENVRIETINDFDMVKFDLVTDLGRVE | 26 |
| | | LAEHVNYDSEGDFKSVEYTDSNIRYNMVDEL | |
| | | CSVFDLTDKPSLMPAIDYVTFAEIIEAVEEMLE | |
| | | A | |
| AcrIF11_pae | Cas3 | MSMELFHGSYEEISEIRDSGVFGGLFGAHEK | 27 |
| | | ETALSHGETLHRIISPLPLTDYALNYEIESAWE | |
| | | VALDVAGGDENVAEAIMAKACESDSNDGWE | |
| | | LQRLRGVLAVRLGYTSVEMEDEHGTTWLCL | |
| | | PGCTVEKI | |
| AcrIF12_pae | Cas3 | MAYEKTWHRDYAAESLKRAETSRWTQDANL | 28 |
| | | EWTQLALECAQVVHLARQVGEELGNEKIIGIA | |
| | | DTVLSTIEAHSQATYRRPCYKRITTAQTHLLA | |
| | | VTLLERFGSARRVANAVWQLTDDEIDQAKA | |
| AcrIF13_mca | Cas3 | MKLLNIKINEFAVTANTEAGDELYLQLPHTPD | 29 |
| | | SQHSINHEPLDDDDFVKEVQEICDEYFGKGD | |
| | | RTLARLSYAGGQAYDSYTEEDGVYTTNTGD | |
| | | QFVEHSYADYYNVEVYCKADLV | |
| AcrIF14_mca | Cas3 | MKKIEMIEISQNRQNLTAFLHISEIKAINAKLAD | 30 |
| | | GVDVDKKSFDEICSIVLEQYQAKQISNKQASE | |
| | | IFETLAKANKSFKIEKFRCSHGYNEIYKYSPD | |
| | | HEAYLFYCKGGQGQLNKLIAENGRFM | |
| AcrIE4-IF7_ | Cas3 | MSTQYTYQQIAEDFRLWSEYVDTAGEMSKD | 31 |
| pae2 | | EFNSLSTEDKVRLQVEAFGEEKSPKFSTKVT | |
| | | TKPDFDGFQFYIEAGRDFDGDAYTEAYGVAV | |
| | | PTNIAARIQAQAAELNAGEWLLVEHEA | |
| AcrIIA1_lmo | Cas9 | MTIKLLDEFLKKHDLTRYQLSKLTGISQNTLK | 32 |
| | | DQNEKPLNKYTVSILRSLSLISGLSVSDVLFEL | |
| | | EDIEKNSDDLAGFKHLLDKYKLSFPAQEFELY | |
| | | CLIKEFESANIEVLPFTFNRFENEEHVNIKKDV | |
| | | CKALENAITVLKEKKNELL | |
| AcrIIA2_lmo | Cas9 | MTLTRAQKKYAEAMHEFINMVDDFEESTPDF | 33 |
| | | AKEVLHDSDYVVITKNEKYAVALCSLSTDECE | |
| | | YDTNLYLDEKLVDYSTVDVNGVTYYINIVETN | |
| | | DIDDLEIATDEDEMKSGNQEIILKSELK | |
| AcrIIA3_lmo | Cas9 | MFNKAEIMKQAWNWFNDSNIWLSDIEWVSY | 34 |
| | | TDKEKSFSVCLKAAWSKAKEEVEESKKESKH | |
| | | IAKSEELKAWNWAERKLGLHFNISDDEKFTS | |
| | | VKDETKINFGLSVWACAMKAVKLHNDLFPQT | |
| | | AA | |
| AcrIIA4_lmo | Cas9 | MNINDLIREIKNKDYTVKLSGTDSNSITQLIIRV | 35 |
| (Acx 105) | | NNDGNEYVISESENESIVEKFISAFKNGWNQ | |
| | | EYEDEEEFYNDMQTITLKSELN | |
| AcrIIA5_sth | Cas9 | MAYGKSRYNSYRKRSFNRSNKQRREYAQE | 36 |
| | | MDRLEKAFENLDGWYLSSMKDSAYKDFGKY | |
| | | EIRLSNHSADNKYHDLENGRLIVNIKASKLNF | |
| | | VDIIENKLDKIIEKIDKLDLDKYRFINATNLEH | |
| | | DIKCYYKGFKTKKEVI | |
| AcrIIA6_sth | Cas9 | MKINDDIKELILEYMSRYFKFENDFYKLPGIKF | 37 |
| | | TDANWQKFKNGGTDIEKMGAARVNAMLDCL | |
| | | FDDFELAMIGKAQTNYYNDNSLKMNMPFYTY | |
| | | YDMFKKQQLLKWLKNNRDDVIGGTGRMYTA | |
| | | SGNYIANAYLEVALESSSLGSGSYMLQMRFK | |
| | | DYSKGQEPIPSGRQNRLEWIENNLENIR | |
| AcrIIA7_meta | Cas9 | MTFGQALESLKRGHLVARKGWNGKGMFIFM | 38 |
| | | RPEDSLPTNMIVNQVKSLPESFKRWVANNH | |
| | | GDSETDRIKFTAYLCMKAADGTIVNGWLASQ | |
| | | TDMLANDWVIVE | |
| AcrIIA8_meta | Cas9 | MSIFTDMIPAELLINEYKKGQSGAKHDNYVSV | 39 |
| | | GRIMVAIYKNNSFKNTGTVKYQDSTHSGITM | |
| | | SKVFIDGKEYRIDIDTQHYEVQDFDTSGRQTT | |
| | | LILKRIDLYG | |
| AcrIIA9_meta | Cas9 | MKGTEHFKQTIKEYLDGRAQTDELFAVSYAK | 40 |
| | | ENKNLDDCITFILNQVKASGCCGMT1DDEVW | |
| | | SLAIHYYDEDNIDVGNPISCGVVVNHKVELTE | |
| | | EEKAQARKEALKAYQEEEMRKIQQRHSKPK | |
| | | PTAKAAQSNQTELSLFDF | |
| AcrIIA10_meta | Cas9 | MDNKFKLRKAINGIEELNFAFDKLTAIDYKTIC | 41 |
| | | RIERKMNGLSVDALADSIIASAGTRKTSSEFRI | |
| | | ACAWWAAVKGTDGLTVDDYDQLSLDDLLELE | |
| | | TFGLLFFVGSLE | |
| AcrIIA11_meta | Cas9 | MADMTLRQFCERYRKGDFLAKDRETQIEAG | 42 |
| | | WYDWFCDDKALAGRLAKIWGILKGITSDYILD | |
| | | NYRVWFKNNCPMVGPLYDDVRFEPLDEEQR | |
| | | DELYFGVAIDDKRREKKYVIFTARNDYENEC | |
| | | GFNNVREVRQFINGWEDELKNEEFYKAREK | |
| | | KRQEMEEANNKFAEIMQRADEILWNLKED | |
| AcrIIA12 | Cas9 | MSKTMYKNDVIELIKNAKTNNEELLFTSVERN | 43 |
| | | TREAATQYFRCPEKHVSDAGVYYGEDFEFD | |
| | | GFEIFEDDLIYTRSYDKEELN | |
| AcrIIA13 | Cas9 | MEVMNKSIEIKDQNNIVLIDSLGQFFTDIENDN | 44 |
| | | NGRYNIDYVLLNEVEHDNGNTYYEVGMYRT | |
| | | EEVPFSDKVTQDNVELLEDKWLQIDQQGESY | |
| | | VESIFFENEEDAREYIKLVLKGHETFEETAKAI | |
| | | GV | |
| AcrIIA14 | Cas9 | LKKTIEKLLNSDLNSNYIAKKTGVEQSTIYRLR | 45 |
| | | TGERQLGKLGLDSAERLYNYQKEIENMKSVK | |
| | | YISNMSKQEKGYRVYVNVVNEDTDKGFLFPS | |
| | | VPKEVIENDKIDELFNFEHHKPYVQKAKSRYD | |
| | | KNGIGYKIVQLDEGFQKFIELNKEKMKENLDY | |
| AcrIIA15 | Cas9 | MRKTIERLLNSELSSNSIAVRTGVSQAVISKL | 46 |
| | | RNGKKELGNLTLNSAEKLFEYQKEMEKVDT | |
| | | WIVYRGRTADMNKSYIAEGSTYEEVYNNFVD | |
| | | KYGYDVLDEDIYEIQLLKKNGENLDDYDVDS | |
| | | DGINNYDKLDEFRESDYVDLEDYDYRELFEN | |
| | | SSSQVYYHEFEITHE | |
| AcrIIA16_lmo | Cas9 | MGYIGTKRSERSQDAIEDYEVPLNHFNKDLIQ | 47 |
| | | AFIDENEAYDTLKTKKVRLWKFVAPRAGATS | |
| | | WHHTGTYYNKTDHYSLEKVADELLQNGDEW | |
| | | EEQFKAYVKEEQETATSEPVFLSVIKVQIWG | |
| | | GSMKRPKLVGHEVVMGVKKEGWLHAVSKAT | |
| | | QSKYKLSANKVEMQKHYSLEDYSALTKDFPE | |
| | | FKAQKRAINKKMKEMYN | |
| AcrIIA16_efa | Cas9 | MGYVGKSRSVRSQIAIDNAEVPLNHITKDYIL | 48 |
| | | TFVTENNIDETLKNESVAMWKFVAKRHGSTS | |
| | | WHHVSKHYNKIDHYDLHDVAEYFSMNYDSL | |
| | | KNDYQNLLDQKRQAKNDLIKNLKLGIIKVQIW | |
| | | GGTKRYPKLEGYESVMGVVKDGWLHTVTLS | |
| | | NQTKYKITGNKIEEITIFELDQYDILTKKFPEFR | |
| | | AMKRKINKEVARLSK | |
| AcrIIA17_efa | Cas9 | MAILNNKGEKISIDCADLISEVEEDILIFGGTFL | 49 |
| | | VYAICSWREIEQVEYISDYVHADNPESYKDEL | |
| | | TTKEYAELKEIYEKDLEELKITKNKQMNLNELL | |
| | | SILTIQNSIT | |
| AcrIIA17_sga | Cas9 | MKISVDSEKLLNEAINDFDIFGEDFNVYAIYSY | 50 |
| | | REDYDFEYISDYVDADEPTRDEFETEEDYQE | |
| | | VMKDFKENLDSLKFTKHKKMTIADLVHELWE | |
| | | QNRIF | |
| AcrIIA18_sma | Cas9 | MKIDTTVTEVKENGKTYLRLLKGNEQLKAVS | 51 |
| | | DKAVAGVNLFPGAKIGSFLVRQDNIVVFPDN | |
| | | KGEFDLDFFNLLNDNFETLVEYAKMADCLDIA | |
| | | FDINEKSYFNMIMWLMKNIDENWSQSPYGES | |
| | | FYSSKDIDWGYKPEGSLRVSDHWNFGQDGE | |
| | | HCPTAEPVDGWAVCKFENGKYHLIKKF | |
| AcrIIA18_sga | Cas9 | MKIDTTVTEVKENGKTYLRLVEGTEQLKAISD | 52 |
| | | KAMAGVNLFPGAKIDSFLVKQDSIVVFPDNK | |
| | | GEFDLDFFKQLDENFDTIAKYARVATCFEEVA | |
| | | FDEKSYFNMIMWLMDNMDENWSQSPYGES | |
| | | FYSSKNIDWGYKPEGSLRVSDHWNFGENGE | |
| | | HCPTAEPVDGWAVCKFENGKYHLIKKF | |
| AcrIIA19_ssi | Cas9 | MKLIVEVEETNYKNLVNYTKLTNESHNILVNR | 53 |
| | | LISEYITKPYELRLDLSERYSNRDLIEFKFMLIE | |
| | | YCKEALQDIKELANSDEAYETDEAFEAVFRQ | |
| | | LFEEVISNPDTVLKAFHSYTSFLEENK | |
| AcrIIA19_sps | Cas9 | MKLIINIEDKNYKYLTELAQQDNTNIGSIVNNLI | 54 |
| | | QTHITDVNESYRSVDKKELDEFSRVMQHYFH | |
| | | EDLASMYDVIGSDEELSTDKQMLKVYKKLYQ | |
| | | DVALRNGIALELFNAYKKG | |
| AcrIIA20_ML1 | Cas9 | MKNYEVTNEVKNLNTQVETIGQAVDLYKEYG | 55 |
| | | SNTIVWSIDKNEDLIDEVTELVAEYAEKGTVIK | |
| AcrIIA21_ML8 | Cas9 | MDYDNENYLIPKILLQDDFYSSLSAKDILVYAV | 56 |
| | | LKDRQIEALEKGWIDTDGSIYLNFKLIELAKMF | |
| | | SCSRTTMIDVMQRLEEVNLIERERVDVFYGY | |
| | | SLPYKTYINEV | |
| AcrIIC1_boe | Cas9 | MANKTYKIGKNAGYDGCGLCLAAISENEAIKV | 57 |
| | | KYLRDICPDYDGDDKAEDWLRWGTDSRVKA | |
| | | AALEMEQYAYTSVGMASCWEFVEL | |
| AcrIIC2_nme | Cas9 | MSKNNIFNKYPTIIHGEARGENDEFVVHTRYP | 58 |
| | | RFLARKSFDDNFTGEMPAKPVNGELGQIGEP | |
| | | RRLAYDSRLGLWLSDFIMLDNNKPKNMEDW | |
| | | LGQLKAACDRIAADDLMLNEDAADLEGWDD | |
| AcrIIC3_nme | Cas9 | MFKRAIIFTSFNGFEKVSRTEKRRLAKIINARV | 59 |
| | | SIIDEYLRAKDTNASLDGQYRAFLFNDESPAM | |
| | | TEFLAKLKAFAESCTGISIDAWEIEESEYVRLP | |
| | | VERRDFLAAANGKEIFKI | |
| AcrIIC4_hpa | Cas9 | MKITSSNFATIATSENFAKLSVLPKNHREPIKG | 60 |
| | | LFKSAVEQFSSARDFFKNENYSKELAEKFNK | |
| | | EAVNEAVEKLQKAIDLAEKQGIQF | |
| AcrIIC5_smu | Cas9 | MNNSIKFHVSYDGTARALFNTKEQAEKYCLV | 61 |
| | | EEINDEMNGYKRKSWEEKLREENCASVQDW | |
| | | VEKNYTSSYSDLFNICEIEVSSAGQLVKIDNT | |
| | | EVDDFVENCYGFTLEDDLEEFNKAKQYLQKF | |
| | | YAECEN | |
| AcrIIC6 | Cas9 | MTESLIHLRVPAATKGRWWRASRAVGLRLTD | 62 |
| | | YITQAVEAYMQQQLTRVAIPDDIEFSDLKLAR | |
| | | DPDGAVSFDWAVIERICHASGLPLEMMRDAP | |
| | | EDNVASLIIGWYQAHRADGGAADPVADDLIA | |
| | | EAMAEDAAGQQFSHQPGRA | |
| AcrIIC7 | Cas9 | MATVTKITLNGQNHYNFGSECSEADAEGYRE | 63 |
| | | WIAQELAENFPGAEIEINEADSTYSVVVEIDD | |
| | | ESYYDEARGLKDDVNVFCIDAWDRCPWDW | |
| | | VS | |
| AcrIIC8 | Cas9 | MYAIRKIQFFYGPTDKKSYVGEEAGGRRELF | 64 |
| | | KTRAEAQARIEDLEEGVYYLAHNESGRPDYK | |
| | | IVWWRGEAQFEHARWMRG | |
| AcrIII-1 | | MNKVYLANAFSINMLTKFPTKVVIDKIDRLEFC | 65 |
| | | ENIDNEDIINSIGHDSTIQLINSLCGTTFQKNRV | |
| | | EIKLEKEDKLYVVQISQRLEEGKILTLEEILKLY | |
| | | ESGKVQFFEIIVD | |
| AcrIIIB1_sis | | MEVKQIKKLNNLPWFLDTYLNKFALDKNFV | 66 |
| | | NCAYYSSRSGMTQEGCVQVMQVGDNFKVD | |
| | | TMREVHGIYFTPHASIISLIYRQKGIRSIDDLKE | |
| | | ILGSLNLSKVSPKHYQLLVKYSNYTIEIYDIYFK | |
| | | GHIYEFPLVSQQGHLNVYNVPEPRNVYLIYYE | |
| | | NNEEKKELNKDLFNEVSEFMIYNHRVTFEKP | |
| | | VLEFKNLQITPGGGALVYVPESMYVKLESSD | |
| | | HQSVEFRPSRDDWLLFSHPRPRRSGND | |
| AcrVA1_mbo | Cas12 | MYEAKERYAKKKMQENTKIDTLTDEQHDALA | 67 |
| (Acx 137) | | QLCAFRHKFHSNKDSLFLSESAFSGEFSFEM | |
| | | QSDENSKLREVGLPTIEWSFYDNSHIPDDSF | |
| | | REWFNFANYSELSETIQEQGLELDLDDDETY | |
| | | ELVYDELYTEAMGEYEELNQDIEKYLRRIDEE | |
| | | HGTQYCPTGFARLR | |
| AcrVA2_mbo | Cas12 | MHHTIARMNAFNKAFANAKDCYKKMQAWHL | 68 |
| | | LNKPKHAFFPMQNTPALDNGLAALYELRGGK | |
| | | EDAHILSILSRLYLYGAWRNTLGIYQLDEEIIK | |
| | | DCKELPDDTPTSIFLNLPDWCVYVDISSAQIA | |
| | | TFDDGVAKHIKGFWAIYDIVEMNGINHDVLDF | |
| | | VVDTDTDDNVYVPQPFILSSGQSVAEVLDYG | |
| | | ASLFDDDTSNTLIKGLLPYLLWLCVAEPDITYK | |
| | | GLPVSREELTRPKHSINKKTGAFVTPSEPFIY | |
| | | QIGERLGSEVRRYQSIIDGEQKRNRPHTKRP | |
| | | HIRRGHWHGYWQGTGQAKEFRVRWQPAVF | |
| | | VNSGRVSS | |
| AcrVA3_mbo | Cas12 | MVGKSKIDWQSIDWTKTNAQIAQECGRAYNT | 69 |
| | | VCKMRGKLGKSHQGAKSPRKDKGISRPQPH | |
| | | LNRLEYQALATAKAKASPKAGRFETNTKAKT | |
| | | WTLKSPDNKTYTFTNLMHFVRTNPHLFDPDD | |
| | | VVWRTKSNGVEWCRASSGLALLAKRKKAPL | |
| | | SWKGWRLISLTKDNK | |
| AcrVA4_mbo | Cas12 | MYEIKLNDTLIHQTDDRVNAFVAYRYLLRRGD | 70 |
| | | LPKCENIARMYYDGKVIKTDVIDHDSVHSDEQ | |
| | | AKVSNNDIIKMAISELGVNNFKSLIKKQGYPFS | |
| | | NGHINSWFTDDPVKSKTMHNDEMYLVVQALI | |
| | | RACIIKEIDLYTEQLYNIIKSLPYDKRPNVVYSD | |
| | | QPLDPNNLDLSEPELWAEQVGECMRYAHND | |
| | | QPCFYIGSTKRELRVNYIVPVIGVRDEIERVM | |
| | | TLEEVRNLHK | |
| AcrVA5_mbo | Cas12 | MKIELSGGYICYSIEEDEVTIDMVEVTTKRQGI | 71 |
| | | GSQLIDMVKDVAREVGLPIGLYAYPQDDSISQ | |
| | | EDLIEFYFSNDFEYDPDDVDGRLMRWS | |
| AcrVIA1_lwa | Cas13 | MEKIKLICLRINNDELITTDKDEWLKFIKRHRG | 72 |
| | | KVSSIEQFNWKIPGNKLQKALEYSFDELYKFK | |
| | | QKENRRETD | |
| AcrVIA2_lwa | Cas13 | MWKCKKCGCDRFYQDITGGISEVLEMDKDG | 73 |
| | | EVLDEIDDVEYGDFSCAKCDNSSSKIQEIAYW | |
| | | DEINGKNKTYLSKDK | |
| AcrVIA3_lwa | Cas13 | MFKEFLEKCLRYGNLYILEETGDRKKVKRISK | 74 |
| | | RHGKVTEASVLLFDSGTKRTTINEIYLNSQGY | |
| | | FIIRDQKRLKLEKFK | |
| AcrVIA4_lwa | Cas13 | MDKANRCLKAKDKILNILEKEEITLDEFNNISK | 75 |
| | | DIAKEYVEKAVLKPKDIAERIINMVKNAKSISF | |
| | | DELASEISEE | |
| AcrVIA5_lwa | Cas13 | MERNFKKVTENTGRKEVFKVMHDKVEIINDF | 76 |
| | | NTNEKREARIIFHDQKIYVILYQNLNFEELKWL | |
| | | NFYILIYGNQSYGKNTFFEFKLNKNNLIYHLQV | |
| | | WNIIENKKFKSKSISLLVKALSSKAGV | |
| AcrVIA6_lwa | Cas13 | MADKVKSIQPGPIFYDVFLVYLRVIGTNLKDW | 77 |
| | | CAPHGVTATNAKSAATGGWNGTKARALRQK | |
| | | MIDEVGEETFLRLYTERLRREAA | |
| AcrVIA7_lwa | Cas13 | MRIIKLYERIIPKTSSTSYISRWEALNIPDENRN | 78 |
| | | TAAWHPRTYLFSYDKDKAINLYNTTNVLGNS | |
| | | GIKKRIIDYPSKREVYIANFPRAIADLVLTMKD | |
| | | YQLSSLHNCCNDFFNEDETEQLYQYLRSIKD | |
| | | NRRVDEFLKYEFTVRYFNDKKF | |
| AcrVIA1_lse | Cas13 | MIYYIKDLKVKGKIFENLMNKEAVEGLITFLKK | 79 |
| | | AEFEIYSRENYSKYNKWFEMWKSPTSSLVF | |
| | | WKNYSFRCHLLFVIEKDGECLGIPASVFESVL | |
| | | QIYLADPFAPDTKELFVEVCNLYECLADVTVV | |
| | | EHFEAEESAWHKLTHNETEVSKRVYSKDDD | |
| | | ELLKYIPEFLDTIATNKKSQKYNQIQGKIQEIN | |
| | | KEIATLYESSEDYIFTEYVSNLYRESAKLEQH | |
| | | SKQILKEELN | |
| Acx 137 | Cas12 | MYEAKERYAKKKMQENTKIDTLTDEQHD | 80 |
| | | ALAQLCAFRHKFHSNKDSLFLSESAFSG | |
| | | EFSFEMQSDENSKLREVGLPTIEWSFYD | |
| | | NSHIPDDSFREWFNFANYSELSETIQEQ | |
| | | GLELDLDDDETYELVYDELYTEAMGEYE | |
| | | ELNQDIEKYLRRIDEEHGTQYCPTGFARL | |
| | | R | |
| Acx 153 | Cas9 | MNINDLIREIKNKDYTVKLSGTDSNSITQLI | 81 |
| (mutant Acr) | | IRVNNDGAEYVISESENESIVEKFISAFKN | |
| | | GWNQEYEDEEEFYNDMQTITLKSELN | |
| Acx 164 | Cas9 | MNINDLIREIKNKAYTVKLSGTDSNSITQLI | 82 |
| (mutant Acr) | | IRVNNDANEYVISESENESIVEKFISAFKN | |
| | | GWNQEYEDEEEFYNDMQTITLKSELN | |
| Acx 162 | Cas9 | MRNLKELVREIEKKGYRVINQTTDLVIDIN | 161 |
| (mutant Acr) | | GNGADYPIKANHYKSILEQFIEIFKNGWN | |
| | | GVYEDEETFYNDMQEIAKNIVLENLEVTY | |
| | | DS | |

TABLE 3
Examples of Cas Effector proteins

| | | | SEQ ID |
|---|---|---|---|
| Name | UniProt | Amino Acid sequence | NO: |
| SaCas9 | J7RUA5 | MKRNYILGLDIGITSVGYGI | 83 |
| | | IDYETRDVIDAGVRLFKEAN | |
| | | VENNEGRRSKRGARRLKRRR | |
| | | RHRIQRVKKLLFDYNLLTDH | |
| | | SELSGINPYEARVKGLSQKL | |
| | | SEEEFSAALLHLAKRRGVHN | |
| | | VNEVEEDTGNELSTKEQISR | |
| | | NSKALEEKYVAELQLERLKK | |
| | | DGEVRGSINRFKTSDYVKEA | |
| | | KQLLKVQKAYHQLDQSFIDT | |
| | | YIDLLETRRTYYEGPGEGSP | |
| | | FGWKDIKEWYEMLMGHCTYF | |
| | | PEELRSVKYAYNADLYNALN | |
| | | DLNNLVITRDENEKLEYYEK | |
| | | FQIIENVFKQKKKPTLKQIA | |
| | | KEILVNEEDIKGYRVTSTGK | |
| | | PEFTNLKVYHDIKDITARKE | |
| | | IIENAELLDQIAKILTIYQS | |
| | | SEDIQEELTNLNSELTQEEI | |
| | | EQISNLKGYTGTHNLSLKAI | |
| | | NLILDELWHTNDNQIAIFNR | |
| | | LKLVPKKVDLSQQKEIPTTL | |
| | | VDDFILSPVVKRSFIQSIKV | |
| | | INAIIKKYGLPNDIIIELAR | |
| | | EKNSKDAQKMINEMQKRNRQ | |
| | | TNERIEEIIRTTGKENAKYL | |
| | | IEKIKLHDMQEGKCLYSLEA | |
| | | IPLEDLLNNPFNYEVDHIIP | |
| | | RSVSFDNSFNNKVLVKQEEN | |
| | | SKKGNRTPFQYLSSSDSKIS | |
| | | YETFKKHILNLAKGKGRISK | |
| | | TKKEYLLEERDINRFSVQKD | |
| | | FINRNLVDTRYATRGLMNLL | |
| | | RSYFRVNNLDVKVKSINGGF | |
| | | TSFLRRKWKFKKERNKGYKH | |
| | | HAEDALIIANADFIFKEWKK | |
| | | LDKAKKVMENQMFEEKQAES | |
| | | MPEIETEQEYKEIFITPHQI | |
| | | KHIKDFKDYKYSHRVDKKPN | |
| | | RELINDTLYSTRKDDKGNTL | |
| | | IVNNLNGLYDKDNDKLKKLI | |
| | | NKSPEKLLMYHHDPQTYQKL | |
| | | KLIMEQYGDEKNPLYKYYEE | |
| | | TGNYLTKYSKKDNGPVIKKI | |
| | | KYYGNKLNAHLDITDDYPNS | |
| | | RNKVVKLSLKPYRFDVYLDN | |
| | | GVYKFVTVKNLDVIKKENYY | |
| | | EVNSKCYEEAKKLKKISNQA | |
| | | EFIASFYNNDLIKINGELYR | |
| | | VIGVNNDLLNRIEVNMIDIT | |
| | | YREYLENMNDKRPPRIIKTI | |
| | | ASKTQSIKKYSTDILGNLYE | |
| | | VKSKKHPQIIKKG | |
| SpCas9 | Q99ZW2 | MDKKYSIGLDIGTNSVGWAV | 84 |
| | | ITDEYKVPSKKFKVLGNTDR | |
| | | HSIKKNLIGALLFDSGETAE | |
| | | ATRLKRTARRRYTRRKNRIC | |
| | | YLQEIFSNEMAKVDDSFFHR | |
| | | LEESFLVEEDKKHERHPIFG | |
| | | NIVDEVAYHEKYPTIEGDLN | |
| | | PDNSDVDKLFIQLVQTYNQL | |
| | | FEENPINASGVDAKAILSAR | |
| | | LSKSRRLENLIAQLPGEKKN | |
| | | GLFGNLIALSLGLTPNFKSN | |
| | | FDLAEDAKLQLSKDTYDDDL | |
| | | DNLLAQIGDQYADLFLAAKN | |
| | | LSDAILLSDILRVNTEITKA | |
| | | PLSASMIKRYDEHHQDLTLL | |
| | | KALVRQQLPEKYKEIFFDQS | |
| | | KNGYAGYIDGGASQEEFYKF | |
| | | IKPILEKMDGTEELLVKLNR | |
| | | EDLLRKQRTFDNGSIPHQIH | |
| | | LGELHAILRRQEDFYPFLKD | |
| | | NREKIEKILTFRIPYYVGPL | |
| | | ARGNSRFAWMTRKSEETITP | |
| | | WNFEEVVDKGASAQSFIERM | |
| | | TNFDKNLPNEKVLPKHSLLY | |
| | | EYFTVYNELTKVKYVTEGMR | |
| | | KPAFLSGEQKKAIVDLLFKT | |
| | | NRKVTVKQLKEDYFKKIECF | |
| | | DSVEISGVEDRFNASLGTYH | |
| | | DLLKIIKDKDFLDNEENEDI | |
| | | LEDIVLTLTLFEDREMIEER | |
| | | LKTYAHLFDDKVMKQLKRRR | |
| | | YTGWGRLSRKLINGIRDKQS | |
| | | GKTILDFLKSDGFANRNFMQ | |
| | | LIHDDSLTFKEDIQKAQVSG | |
| | | QGDSLHEHIANLAGSPAIKK | |
| | | GILQTVKVVDELVKVMGRHK | |
| | | PENIVIEMARENQTTQKGQK | |
| | | NSRERMKRIEEGIKELGSQI | |
| | | LKEHPVENTQLQNEKLYLYY | |
| | | LQNGRDMYVDQELDINRLSD | |
| | | YDVDHIVPQSFLKDDSIDNK | |
| | | VLTRSDKNRGKSDNVPSEEV | |
| | | VKKMKNYWRQLLNAKLITQR | |
| | | KFDNLTKAERGGLSELDKAG | |
| | | FIKRQLVETRQITKHVAQIL | |
| | | DSRMNTKYDENDKLIREVKV | |
| | | ITLKSKLVSDFRKDFQFYKV | |
| | | REINNYHHAHDAYLNAVVGT | |
| | | ALIKKYPKLESEFVYGDYKV | |
| | | YDVRKMIAKSEQEIGKATAK | |
| | | YFFYSNIMNFFKTEITLANG | |
| | | EIRKRPLIETNGETGEIVWD | |
| | | KGRDFATVRKVLSMPQVNIV | |
| | | KKTEVQTGGFSKESILPKRN | |
| | | SDKLIARKKDWDPKKYGGFD | |
| | | SPTVAYSVLVVAKVEKGKSK | |
| | | KLKSVKELLGITIMERSSFE | |
| | | KNPIDFLEAKGYKEVKKDLI | |
| | | IKLPKYSLFELENGRKRMLA | |
| | | SAGELQKGNELALPSKYVNF | |
| | | LYLASHYEKLKGSPEDNEQK | |
| | | QLFVEQHKHYLDEIIEQISE | |
| | | FSKRVILADANLDKVLSAYN | |
| | | KHRDKPIREQAENIIHLFTL | |
| | | TNLGAPAAFKYFDTTIDRKR | |
| | | YTSTKEVLDATLIHQSITGL | |
| | | YETRIDLSQLGGD | |
| NmeCas9 | C6SFU3 | MAAFKPNPINYILGLDIGIA | 85 |
| | | SVGWAMVEIDEDENPICLID | |
| | | LGVRVFERAEVPKTGDSLAA | |
| | | ARRLARSVRRLTRRRAHRLL | |
| | | RARRLLKREGVLQAADFDEN | |
| | | GLIKSLPNTPWQLRSAALDR | |
| | | KLTPLEWSAVLLHLIKHRGY | |
| | | LSQRKNEGETADKELGALLK | |
| | | GVADNAHALQTGDFRTPAEL | |
| | | ALNKFEKESGHIRNRRGDYS | |
| | | HTFSRKDLQAELDLLFEKQK | |
| | | EFGNPHISDDLKEGIETLLM | |
| | | TQRPALSGDAVQKMLGHCTF | |
| | | EPTEPKAAKNTYTAERFVWL | |
| | | TKLNNLRILEQGSERPLTDT | |
| | | ERATLMDEPYRKSKLTYAQA | |
| | | RKLLELDDTAFFKGLRYGKD | |
| | | NAEASTLMEMKAYHAISRAL | |
| | | EKEGLKDKKSPLNLSPELQD | |
| | | EIGTAFSLFKTDEDITGRLK | |
| | | DRVQPEILEVLLKHISFDKF | |
| | | VQISLKALRRIVPLMEQGKR | |
| | | YDEACAEIYGDHDGKKNTEE | |
| | | KIYLPPIPADEIRNPVVLRA | |
| | | LSQARKVINAVVRRYGSPAR | |
| | | IHIETAREVGKSFKDRKEIE | |
| | | KRQEENRKDREKAAAKFREY | |
| | | FPNFVGEPKSKDILKLRLYE | |
| | | QQHGKCLYSYHLRKKLVDST | |
| | | DKADLRLIYLALAHMIKFRG | |
| | | HFLIGKEINLGRLNEKGYVE | |
| | | IDHALPFSRTWDDSFNNKVL | |
| | | VLGSENQNKGNQTPYEYFNG | |
| | | KDNSREWQEFKARVETSRFP | |
| | | RSKKQRILLQKFDEEGFKER | |
| | | NLNDTRYVNRFLCQFVADHM | |
| | | LLTGKGKRRVFASNGQITNL | |
| | | LRGFWGLRKVRAENNRHHAL | |
| | | DAVVVACSTVAMQQKITRFV | |
| | | RYKEMNAFDGKTIDKETGEV | |
| | | LHQKTHFPQPWEFFAQEVMI | |
| | | RVFGKPDGKPEFEEADTPEK | |
| | | LRTLLAEKLSSRPEAVHEYV | |
| | | TPLFVSRAPNRKMSGQGHME | |
| | | TVKSAKRLDEGVSVLRVPLT | |
| | | QLKLKDLEKMVNREREPKLY | |
| | | EALKARLEAHKDDPAKAFAE | |
| | | PFYKYDKAGNRTQQVKAVRV | |
| | | EQVQKTGVWWVRNHNGIADN | |
| | | ATMVRVDVFEKAGKYYLVPI | |
| | | YSWQVAKGILPDRAVVAYAD | |
| | | EEGWTVIDESFRFKFVLYSN | |
| | | DLIKVQLKKDSFLGYFSGLD | |
| | | RATGAISLREHDLEKSKGKD | |
| | | GMHRIGVKTALSFQKYQIDE | |
| | | MGKEIRLCRLKKRPPVR | |
| Cas12a | U2UMQ6 | MTQFEGFTNLYQVSKTLRFE | 86 |
| | | LIPQGKTLKHIQEQGFIEED | |
| | | KARNDHYKELKPIIDRIYKT | |
| | | YADQCLQLVQLDWENLSAAI | |
| | | DSYRKEKTEETRNALIEEQA | |
| | | TYRNAIHDYFIGRTDNLTDA | |
| | | INKRHAEIYKGLFKAELFNG | |
| | | KVLKQLGTVTTTEHENALLR | |
| | | SFDKFTTYFSGFYENRKNVF | |
| | | SAEDISTAIPHRIVQDNFPK | |
| | | FKENCHIFTRLITAVPSLRE | |
| | | HFENVKKAIGIFVSTSIEEV | |
| | | FSFPFYNQLLTQTQIDLYNQ | |
| | | LLGGISREAGTEKIKGLNEV | |
| | | LNLAIQKNDETAHIIASLPH | |
| | | RFIPLFKQILSDRNTLSFIL | |
| | | EEFKSDEEVIQSFCKYKTLL | |
| | | RNENVLETAEALFNELNSID | |
| | | LTHIFISHKKLETISSALCD | |
| | | HWDTLRNALYERRISELTGK | |
| | | ITKSAKEKVQRSLKHEDINL | |
| | | QEIISAAGKELSEAFKQKTS | |
| | | EILSHAHAALDQPLPTTLKK | |
| | | QEEKEILKSQLDSLLGLYHL | |
| | | LDWFAVDESNEVDPEFSARL | |
| | | TGIKLEMEPSLSFYNKARNY | |
| | | ATKKPYSVEKFKLNFQMPTL | |
| | | ASGWDVNKEKNNGAILFVKN | |
| | | GLYYLGIMPKQKGRYKALSF | |
| | | EPTEKTSEGFDKMYYDYFPD | |
| | | AAKMIPKCSTQLKAVTAHFQ | |
| | | THTTPILLSNNFIEPLEITK | |
| | | EIYDLNNPEKEPKKFQTAYA | |
| | | KKTGDQKGYREALCKWIDFT | |
| | | RDFLSKYTKTTSIDLSSLRP | |
| | | SSQYKDLGEYYAELNPLLYH | |
| | | ISFQRIAEKEIMDAVETGKL | |
| | | YLFQIYNKDFAKGHHGKPNL | |
| | | HTLYWTGLFSPENLAKTSIK | |
| | | LNGQAELFYRPKSRMKRMAH | |
| | | RLGEKMLNKKLKDQKTPIPD | |
| | | TLYQELYDYVNHRLSHDLSD | |
| | | EARALLPNVITKEVSHEIIK | |
| | | DRRFTSDKFFFHVPITLNYQ | |
| | | AANSPSKFNQRVNAYLKEHP | |
| | | ETPIIGIDRGERNLIYITVI | |
| | | DSTGKILEQRSLNTIQQFDY | |
| | | QKKLDNREKERVAARQAWSV | |
| | | VGTIKDLKQGYLSQVIHEIV | |
| | | DLMIHYQAVVVLENLNFGFK | |
| | | SKRTGIAEKAVYQQFEKMLI | |
| | | DKLNCLVLKDYPAEKVGGVL | |
| | | NPYQLTDQFTSFAKMGTQSG | |
| | | FLFYVPAPYTSKIDPLTGFV | |
| | | DPFVWKTIKNHESRKHFLEG | |
| | | FDFLHYDVKTGDFILHFKMN | |
| | | RNLSFQRGLPGFMPAWDIVF | |
| | | EKNETQFDAKGTPFIAGKRI | |
| | | VPVIENHRFTGRYRDLYPAN | |
| | | ELIALLEEKGIVFRDGSNIL | |
| | | PKLLENDDSHAIDTMVALIR | |
| | | SVLQMRNSNAATGEDYINSP | |
| | | VRDLNGVCFDSRFQNPEWPM | |
| | | DADANGAYHIALKGQLLLNH | |
| | | LKESKDLKLQNGISNQDWLA | |
| | | YIQELRN | |

In some embodiments, a subject Acr protein inhibits a Cas protein from a Type II or type V CRISPR system. In some cases, a subject Acr protein is selected from the group consisting of: AcrIIA1, AcrIIA2, AcrIIA3, AcrIIA4, AcrIIA5, AcrIIA6, AcrIIA7, AcrIIA8, AcrIIA9, AcrIIA10, AcrIIA11, AcrIIA12, AcrIIA13, AcrIIA14, AcrIIA15, AcrIIA16, AcrIIA17, AcrIIA18, AcrIIA19, AcrIIC1, AcrIIC2, AcrIIC3, AcrIIC4, AcrIIC5, AcrVA1, AcrVA2, AcrVA3, AcrVA4, and AcrVA5.

In some embodiments, a subject Acr protein inhibits a Cas protein from a Type II CRISPR system. Thus, in some cases, a subject Acr protein is selected from the group consisting of: AcrIIA1, AcrIIA2, AcrIIA3, AcrIIA4, AcrIIA5, AcrIIA6, AcrIIA7, AcrIIA8, AcrIIA9, AcrIIA10, AcrIIA11, AcrIIA12, AcrIIA13, AcrIIA14, AcrIIA15, AcrIIA16, AcrIIA17, AcrIIA18, AcrIIA19, AcrIIC1, AcrIIC2, AcrIIC3, AcrIIC4, and AcrIIC5.

In some embodiments, a subject Acr protein inhibits a Cas protein from a Type V CRISPR system. Thus, in some cases, a subject Acr protein is selected from the group consisting of: AcrVA1, AcrVA2, AcrVA3, AcrVA4, and AcrVA5.

In some embodiments, a subject Acr protein inhibits a Cas protein from a Type I or type III CRISPR system. Thus, in some cases, a subject Acr protein is selected from the group consisting of: AcrIC1, AcrID1, AcrIE1, AcrIE2, AcrIE3, AcrIE4, AcrIE4-IF7, AcrIE5, AcrIE6, AcrIE7, AcrIF1, AcrIF2, AcrIF3, AcrIF4, AcrIF5, AcrIF6, AcrIF7, AcrIF8, AcrIF9, AcrIF10, AcrIF11, AcrIF12, AcrIF13, AcrIF14, and AcrIIIB1.

In some embodiments, a subject Acr protein inhibits a Cas protein from a Type I

CRISPR system. Thus, in some cases, a subject Acr protein is selected from the group consisting of: AcrIC1, AcrID1, AcrIE1, AcrIE2, AcrIE3, AcrIE4, AcrIE4-IF7, AcrIE5, AcrIE6, AcrIE7, AcrIF1, AcrIF2, AcrIF3, AcrIF4, AcrIF5, AcrIF6, AcrIF7, AcrIF8, AcrIF9, AcrIF10, AcrIF11, AcrIF12, AcrIF13, and AcrIF14.

In some cases, the Cas protein is a Cas 9 protein, and the Acr protein is selected from the group consisting of: AcrIIA1, AcrIIA2, AcrIIA3, AcrIIA4, AcrIIA5, AcrIIA6, AcrIIA7, AcrIIA8, AcrIIA9, AcrIIA10, AcrIIA11, AcrIIA12, AcrIIA13, AcrIIA14, AcrIIA15, AcrIIA16, AcrIIA17, AcrIIA18, and AcrIIA19. In some cases, the Cas 9 protein is NmeCas9 and the Acr protein is selected from the group consisting of Acr-IIC1, Acr-IIC2, Acr-IIC3, Acr-IIC4, and Acr-IIC5. In some cases, the case protein is a Cas 12 protein (e.g., Cas12a) and the Acr is AcrVA2 or AcrVA4.

In some cases, the Acr protein included in the coordinate delivery system is a wildtype Acr amino acid sequence. In some cases, the Acr protein includes one or more amino acid replacements as compared to the wildtype Acr sequence. For example, in some cases the coordinated delivery system includes a Cas protein (e.g., a wildtype Cas protein), and an Acr protein with one or more amino acid replacements (“modified Acr protein”) and the ratio of the Cas and modified Acr proteins are controlled using a translation element described herein.

In some such cases, the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with wild type Acr (see, e.g., Table 2, SEQ ID NOs: 1-79). In some such cases, the Acr protein comprises an amino acid sequence having 80% or more sequence identity (e.g., 85% or more, 90% or more, 95% or more, 97% or more, 98% or more, 99% or more, or 100% sequence identity) with wild type Acr. In some such cases, the Acr protein comprises an amino acid sequence having 90% or more sequence identity (e.g., 95% or more, 97% or more, 98% or more, 99% or more, or 100% sequence identity) with wild type Acr. In some such cases, the Acr protein comprises an amino acid sequence having 95% or more sequence identity (e.g., 97% or more, 98% or more, 99% or more, or 100% sequence identity) with wild type Acr. In some such cases, the Acr protein comprises a wild type Acr amino acid sequence.

In some cases, the Cas protein comprises a SpCas9 (Streptococcus. pyogenes Cas9) and the modified Acr Protein is AcrIIA2 with an amino acid replacement at one or more positions, for example one or more selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122 and K123. In some cases, the one or more positions are replaced with an alanine or with an arginine. In some cases, the one or more positions are replaced with a conservative amino acid change, such as one that preserves charge or size or shape of the amino acid. In some cases, the one or more positions are replaced with a non-conservative amino acid change, such as one that alters charge, size and/or shape of the amino acid. In combination any of the above amino acid replacement embodiments, in some case the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with wild type AcrIIA2. In some such cases, the Acr protein comprises an amino acid sequence having 80% or more sequence identity (e.g., 85% or more, 90% or more, 95% or more, 97% or more, 98% or more, 99% or more, or 100% sequence identity) with wild type AcrIIA2. In some such cases, the Acr protein comprises an amino acid sequence having 90% or more sequence identity (e.g., 95% or more, 97% or more, 98% or more, 99% or more, or 100% sequence identity) with wild type AcrIIA2. In some such cases, the Acr protein comprises an amino acid sequence having 95% or more sequence identity (e.g., 97% or more, 98% or more, 99% or more, or 100% sequence identity) with wild type AcrIIA2.

In some cases, the Cas nuclease comprises a SpCas9 and the modified Acr Protein is an AcrIIA4 with an amino acid replacement at one or more positions, for example one or more selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87. In some cases, the one or more positions are replaced with an alanine or with an arginine. In some cases, the one or more positions are replaced with a conservative amino acid change, such as one that preserves charge or size or shape of the amino acid. In some cases, the one or more positions are replaced with a non-conservative amino acid change, such as one that alters charge, size and/or shape of the amino acid. In some cases, the AcrIIA4 comprises one or more amino acid mutations (replacements) selected from: D14A, G38A, N39A, and any combination thereof (e.g., in some cases N39A or the amino acid replacements D14A and G38A). In combination any of the above amino acid replacement embodiments, in some case the Acr protein comprises an amino acid sequence having 70% or more sequence identity (e.g., 75% or more, 80% or more, 85% or more, 90% or more, 95% or more, 97% or more, 98% or more, 99% or more, or 100% sequence identity) with wild type AcrIIA4. In some such cases, the Acr protein comprises an amino acid sequence having 80% or more sequence identity (e.g., 85% or more, 90% or more, 95% or more, 97% or more, 98% or more, 99% or more, or 100% sequence identity) with wild type AcrIIA4. In some such cases, the Acr protein comprises an amino acid sequence having 90% or more sequence identity (e.g., 95% or more, 97% or more, 98% or more, 99% or more, or 100% sequence identity) with wild type AcrIIA4. In some such cases, the Acr protein comprises an amino acid sequence having 95% or more sequence identity (e.g., 97% or more, 98% or more, 99% or more, or 100% sequence identity) with wild type AcrIIA4.

In some cases, the Cas effector protein included in a subject coordinated delivery system includes a wildtype amino acid sequence (see, e.g., the example Cas Effector proteins of Table 3). As noted above, in some cases, the Cas effector protein is a variant (is modified/mutated) (i.e., includes one or more amino acid mutations such as substitution(s), insertion(s), deletion(s) relative to a wildtype Cas effector protein). See, e.g., Kleinstiver, B. P. et al. Nature 529, 490-495 (2016); Slaymaker, I. M. et al., Science 351, 84-88 (2016); as well as U.S. Pat. Nos. 11,124,783; 11,098,297; 11,091,798; 11,060,078; and 11,060,115; all of which are incorporated herein by reference. For example, in some cases the coordinated delivery system includes an Acr protein (as described above) and a Cas effector protein with one or more amino acid mutations relative to the wild type protein. In some such cases, the Cas effector protein comprises an amino acid sequence having 70% or more sequence identity (e.g., 75% or more, 80% or more, 85% or more, 90% or more, 95% or more, 97% or more, 98% or more, 99% or more, or 100% sequence identity) with a wild type Cas Effector protein of Table 3 (any one of SEQ ID Nos: 83-86). In some such cases, the Cas Effector protein comprises an amino acid sequence having 80% or more sequence identity (e.g., 85% or more, 90% or more, 95% or more, 97% or more, 98% or more, 99% or more, or 100% sequence identity) with a wild type Cas Effector protein of Table 3 (any one of SEQ ID Nos: 83-86). In some such cases, the Cas Effector protein comprises an amino acid sequence having 90% or more sequence identity (e.g., 95% or more, 97% or more, 98% or more, 99% or more, or 100% sequence identity) with a wild type Cas Effector protein of Table 3 (any one of SEQ ID Nos: 83-86). In some such cases, the Cas Effector protein comprises an amino acid sequence having 95% or more sequence identity (e.g., 97% or more, 98% or more, 99% or more, or 100% sequence identity) with a wild type Cas Effector protein of Table 3 (any one of SEQ ID Nos: 83-86). In some such cases, the Cas Effector protein comprises an amino acid sequence of Table 3 (any one of SEQ ID Nos: 83-86).

### Translational Control Elements

The present disclosure provides coordinated delivery systems (and methods of using such systems) that includes delivery of one or more nucleic acids, where the nucleic acids encode an Acr protein and a Cas effector protein—where the Acr protein is an inhibitor of the Cas protein (e.g., Cas effector protein). In some cases, both coding sequences are present on the same nucleic acid (e.g., vector) and in some cases they are present on separate nucleic acids (e.g., separate vectors).

Subject nucleic acids also include a translational control element that is operably linked to the Acr protein or the Cas protein (e.g., Cas effector protein), or both—in order to achieve a proper balance (expression level ratio) between the two proteins. For example, in some cases a subject nucleic acid includes a translational control element that is operably linked to (and therefore regulates/modulates translation of) a sequence encoding the Acr protein. In some cases, a subject nucleic acid includes a translational control element that is operably linked to (and therefore regulates/modulates translation of) a sequence encoding the Cas protein. In some cases, the sequence encoding the Acr protein and the sequence encoding the Cas protein are both operably linked to a translational control element, e.g., independently (in which case the two control elements can be the same or different).

In some cases, the sequence encoding the Acr protein and the sequence encoding the Cas protein are both operably linked to the same translational control element (e.g., IRES element, 2A peptide encoding sequence) such that the sequences are part of a polycistronic transcript. As such, in some cases a subject translational control element is a polycistronic linker. In other words, in some cases the translational control element promotes (causes) the production of independent gene products (e.g., the Acr protein and the Cas effector protein) from the same transcript.

Thus, in some cases a subject translational control element links a first protein coding sequence (e.g., a sequence encoding an Acr protein) with a second protein coding sequence (e.g., a sequenced encoding a Cas effector protein) such that the first and second proteins (e.g., the Acr protein and Cas effector protein) are encoded by a polycistronic sequence. In such cases, both protein sequences would therefore both be operably linked to the same promotor, and the RNA transcribed therefrom would include both protein-coding sequences in addition to the sequence encoded by the translational control element.

As described in more detail below, in some cases more than one translational control element (e.g., IRES element, 2A peptide, non-AUG start codon) is used to control expression of a given protein (e.g., an Acr protein, a Cas effector protein such as Cas9 or Cas12a). Any convenient combination of translational control elements can be used.

2A Peptide

One non-limiting example of a translational control element that can function as a polycistronic linker and facilitate the production of separate protein products (e.g., two separate proteins) from the same single RNA transcript is a 2A peptide sequence.

By a “2A peptide” it is meant a small peptide sequence (usually 18-25 amino acids although several of such sequences can be placed in tandem) that allows for expression (translation) of discrete protein products from a single RNA transcript (e.g., through a self-“cleaving” event often referred to as “ribosome skipping”—although the disclosure herein does not rely on and is not bound by the mechanism of action), even though the separate proteins are encoding as part of the same open reading frame (ORF). 2A peptides are readily identifiable by their consensus motif (DXEXNPGP, sometimes described as DVEXNPGP) and their ability to promote protein cleavage/skipping. Any convenient 2A peptide sequence may be used in a subject nucleic acid. Examples of 2A peptides include, but are not limited to 2A peptides from a virus such as foot-and-mouth disease virus (F2A), equine Rhinitis A virus (E2A), porcine teschovirus-1 (P2A) or Thosea asigna virus (T2A). See, e.g., Szymczak-Workman, A. et al. “Design and Construction of 2A Peptide-Linked Multicistronic Vectors”. Cold Spring Harb Protoc. 2012 Feb. 1; 2012(2):199-204; Liu et al., Sci Rep. 2017; 7: 2193; Kim et al., PLOS One 6:e18556, 2011; and U.S. Pat. Nos. 10,738,325; 9,655,956; 10,577,417; the disclosures of which, as they relate to 2A peptides, are incorporated herein by reference.

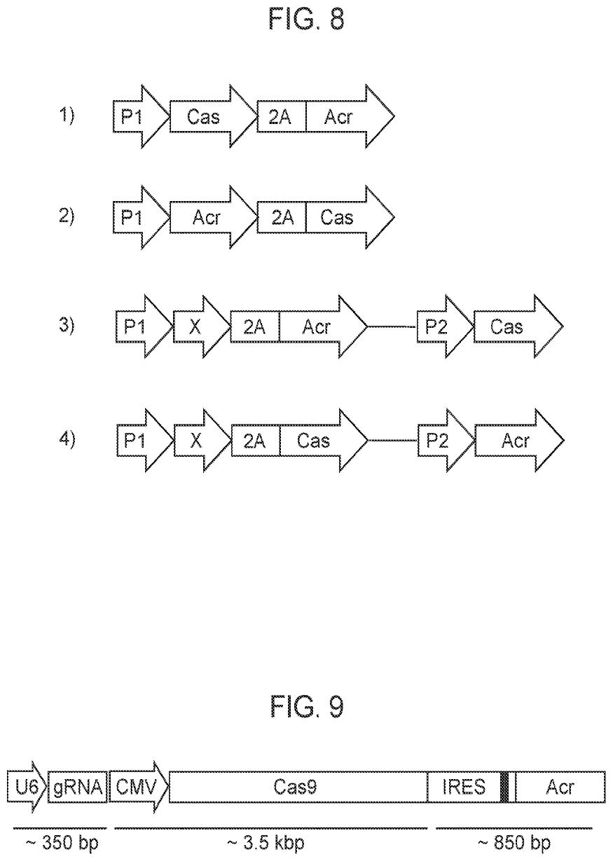

Typically, a subject 2A peptide coding sequence will be positioned in a subject nucleic acid so as to regulate the expression (translation) of a subject protein (e.g., Acr protein or Cas protein)—and as such will be positioned 5′ of (usually immediately 5′ of) and in frame with the protein coding sequence which it regulates. FIG. 8 provides non-limiting illustrative examples of embodiments in which a 2A peptide coding sequence is positioned in different ways. In some cases, a 2A peptide sequence is position 5′ of (and usually immediately 5′ of) a Cas protein coding sequence. In some cases, a 2A peptide sequence is position 5′ of (and usually immediately 5′ of) an Acr protein coding sequence.

In some cases, the Cas-encoding sequence and the Acr-encoding sequence are operably linked to the same promoter (encoded by a polycistronic sequence) and are positioned in tandem, with a 2A peptide sequence positioned between them (see, e.g., FIG. 8, first and second examples). In some such cases, the Cas protein encoding sequence is positioned 5′ of the Acr encoding sequence, and the 2A peptide encoding sequence is therefore 3′ of the Cas sequence and 5′ of the Acr sequence. In other such cases, the Acr protein encoding sequence is positioned 5′ of the Cas encoding sequence, and the 2A peptide encoding sequence is therefore 3′ of the Acr sequence and 5′ of the Cas sequence.

In some cases, the Cas-encoding sequence and the Acr-encoding sequence are operably linked to a first promoter and a second promoter, respectively, such that they are transcribed as separate transcripts (see, e.g., FIG. 8, third and fourth examples). The first and second promoters (labeled “P1” and “P2” in the figure) can be different from one another or can be the same (i.e., can be a copy of the same promoter). In some such cases, the 2A peptide sequence regulates (and is therefore positioned 5′ of) the Acr sequence. In other cases, the 2A peptide sequence regulates (and is therefore positioned 5′ of) the Cas sequence. In some cases, in which the Cas-encoding sequence and the Acr-encoding sequence are transcribed as separate sequences, each one is regulated by a 2A peptide sequence (and each is therefore positioned 3′ of the 2A peptide sequence).

In some embodiments in which the Cas-encoding sequence and the Acr-encoding sequence are transcribed as separate sequences, a “spacer” protein coding sequence is used 5′ of the 2A peptide sequence such that the ‘spacer’ sequence is transcribed as part of a polycistronic sequence with the protein sequence being regulated. For example, in the third example of FIG. 8, a Cas protein coding sequence and an Acr protein coding sequence are operably linked to different promoters (P1 and P2). A spacer sequence (labeled as “X” in the figure) is positioned 5′ of a 2A peptide sequence, which is 5′ of an Acr coding sequence—and therefore the spacer sequence and the Acr sequence are transcribed as part of the same RNA. However, presence of the 2A peptide sequence results in production of the Acr protein as a separate protein. Likewise, in the fourth example of FIG. 8, a Cas protein coding sequence and an Acr protein coding sequence are again operably linked to different promoters (P1 and P2). In this example, a spacer sequence (labeled as “X” in the figure) is positioned 5′ of a 2A peptide sequence, which is 5′ of a Cas coding sequence—and therefore the spacer sequence and the Cas sequence are transcribed as part of the same RNA. However, presence of the 2A peptide sequence in this RNA results in the production of the Cas protein as a separate protein.

A ‘spacer’ protein can be any desired sequence—as its purpose is to simply provide a sequence to be translated that is 5′ of (N-terminal to) the 2A peptide sequence. A spacer sequence can be any convenient length, from very short to encoding an entire protein sequence. In some cases, the spacer is 2 or more amino acids long (e.g., 3 or more, 4 or more 5 or more, 10 or more, or 20 or more amino acids). In some cases, the spacer has a length of from 1 to 100 amino acids (e.g., 1 to 80, 1 to 50, 1 to 40, 1 to 30, 1 to 20, 1 to 10, 2 to 100, 2 to 80, 2 to 50, 2 to 40, 2 to 30, 2 to 20, 2 to 10, 5 to 100, 5 to 80, 5 to 50, 5 to 40, 5 to 30, 5 to 20, or 5 to 10 amino acids). Examples of spacer sequence include, but are not limited to: linker sequences, repeated single amino acids (e.g., AAAA), random sequences, fragments of proteins, and marker proteins (e.g., a fluorescent protein such as GFP, YFP, CFP, RFP, and the like, a drug selectable protein marker, an enzyme such as beta-galactosidase, etc.).

Examples of 2A peptide sequences include, but are not limited to:

(P2A)
(SEQ ID NO: 133)
ATNFSLLKQAGDVEENPGP
(E2A)
(SEQ ID NO: 134)
QCTNYALLKLAGDVESNPGP
(F2A)
(SEQ ID NO: 135)
VKQTLNFDLLKLAGDVESNPGP
(T2A)
(SEQ ID NO: 136)
EGRGSLLTCGDVEENPGP
(EZA-F2A)
(SEQ ID NO: 137)
QCTNYALLKLAGDVESNPGPVKQTLNFDLLKLAGDVESNPGP
(T2A-E2A-F2A)
(SEQ ID NO: 138)
EGRGSLLTCGDVEENPGPQCTNYALLKLAGDVESNPGPVKQTL
NFDLLKLAGDVESNPGP

2A peptide sequences can be used in tandem, and multiple different 2A peptide sequences can be positioned one after another, in any desired combination (see “E2A-F2A” and “T2A-E2A-F2A” above as non-limiting examples). Thus, in some cases a 2A peptide sequence is selected from the group consisting of: P2A, F2A, E2A, T2A, and any combination thereof. In some embodiments, a translational control element encodes 2 or more 2A peptides in tandem (e.g., 3 or more, 4 or more, or 5 or more). In some embodiments, a translational control element encodes 2, 3, 4, or 5 2A peptides in tandem. In some embodiments, a translational control element encodes one 2A peptide.

In some cases, a 2A peptide sequence comprises an amino acid sequence having 70% or more sequence identity (e.g., 75% or more, 80% or more, 85% or more, 90% or more, 95% or more, 97% or more, 98% or more, 99% or more, or 100% sequence identity) with the amino acid sequence set forth in any one of SEQ ID NOs: 133-138. In some cases, a 2A peptide sequence comprises an amino acid sequence having 90% or more (e.g., 95% or more, 97% or more, 98% or more, 99% or more, or 100% sequence identity) with the amino acid sequence set forth in any one of SEQ ID NOs: 133-138. In some cases, a 2A peptide sequence comprises an amino acid sequence having 95% or more (e.g., 97% or more, 98% or more, 99% or more, or 100% sequence identity) with the amino acid sequence set forth in any one of SEQ ID NOs: 133-138. In some cases, a 2A peptide sequence comprises an amino acid sequence having the amino acid sequence set forth in any one of SEQ ID NOs: 133-138.

IRES

One non-limiting example of a translational control element that can function as a polycistronic linker and facilitate the production of separate protein products (e.g., two separate proteins) from the same single RNA transcript is an internal ribosome entry site (IRES) sequence. By an “internal ribosome entry site,” or “IRES” it is meant a nucleotide sequence that allows for the initiation of protein translation within the interior of a messenger RNA (mRNA) sequence (i.e., downstream of the first start codon). For example, when an IRES segment is located between two open reading frames in a bicistronic eukaryotic mRNA molecule, it can drive translation of the downstream protein-coding region independently of the 5′-cap structure at the 5′ end of the mRNA molecule, i.e. in front of the upstream protein coding region. In such a setup, both proteins are produced in the cell. The protein located in the first cistron is synthesized by the cap-dependent initiation mechanism, while translation initiation of the second protein is directed by the IRES segment located in the intercistronic spacer region between the two protein coding regions. IRESs have been isolated from viral genomes and cellular genomes. Artificially engineered IRESs are also known in the art. One of ordinary skill in the art will recognize that the sequences described herein as IRES sequences, which function as part of RNA molecules, will have correlative sequences in the encoding DNA molecules, e.g., RNA sequence 5′-uuacuggc-3′ would correspond to DNA sequence 5′-ttactggc-3′, and vice versa”. The term “IRES sequence” is used herein to refer to either sequence.

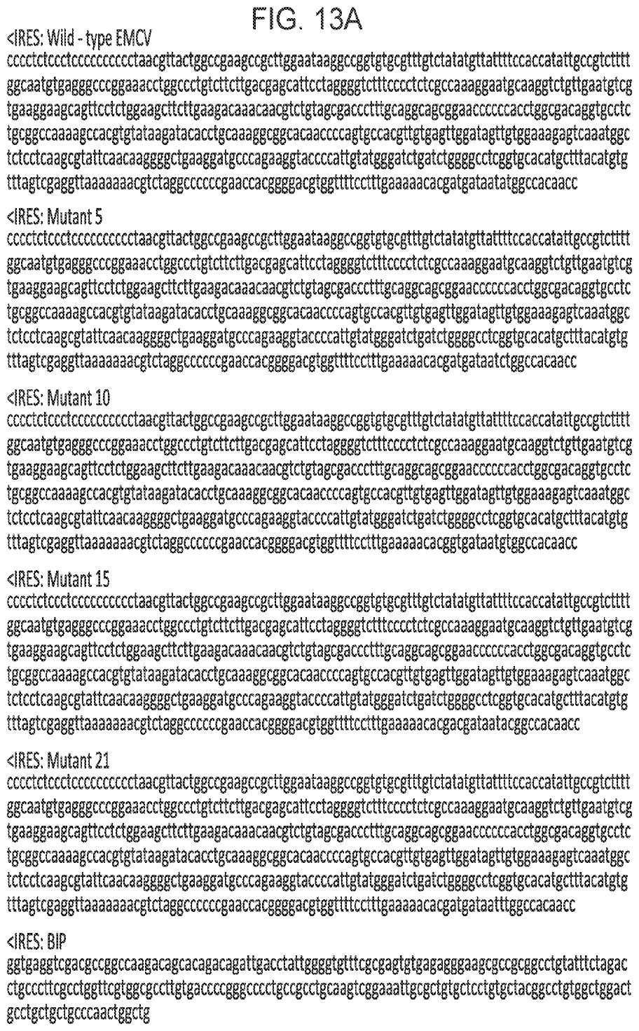

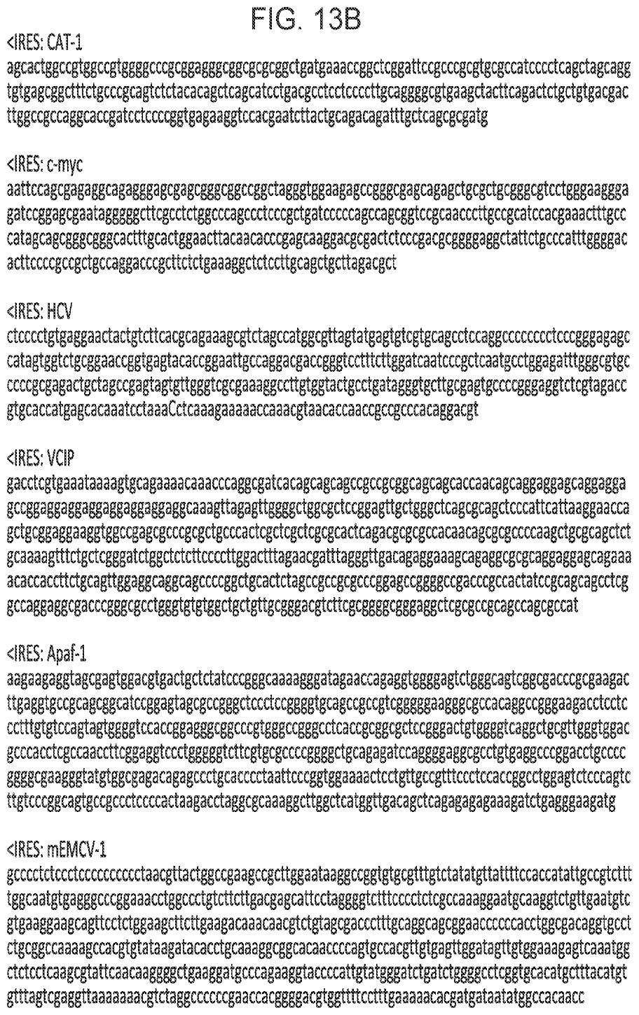

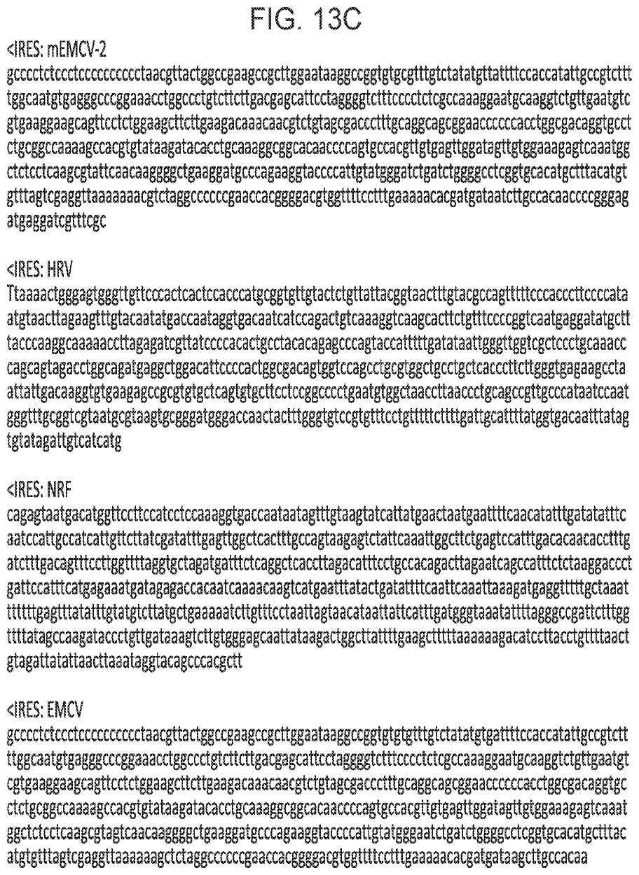

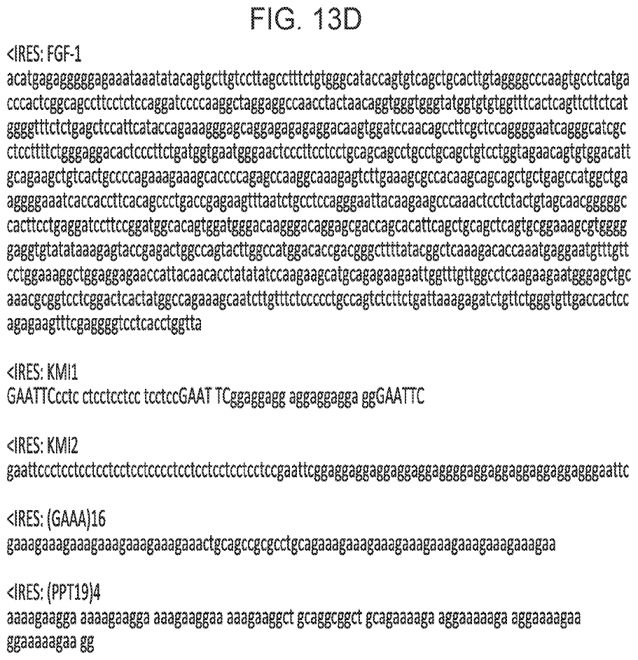

Any convenient IRES may be employed in the subject compositions and methods. Examples of IRES sequences include but are not limited to those listed in FIG. 13A-13D (SEQ ID NOs: 139-159). One of ordinary skill in the art will recognize that when a subject system is to be used for expressing Cas and Acr proteins in non-animal cells (e.g., plants/plant cells), they should select a convenient IRES sequence appropriate for the desired cell type (e.g., an IRES from Triticum mosaic virus (TriMV)). See, e.g., Urwin et al., Plant J. 2000 December; 24(5):583-9 as well U.S. Pat. Nos. 8,772,465; 9,879,271, each of which is incorporated by reference with respect to teachings related to using IRES sequences in plant.

In some cases, an IRES sequence comprises a nucleotide sequence having 70% or more sequence identity (e.g., 75% or more, 80% or more, 85% or more, 90% or more, 95% or more, 97% or more, 98% or more, 99% or more, or 100% sequence identity) with the nucleotide sequence set forth in any one of SEQ ID NOs: 139-159. In some cases, an IRES sequence comprises a nucleotide sequence having 90% or more (e.g., 95% or more, 97% or more, 98% or more, 99% or more, or 100% sequence identity) with the nucleotide sequence set forth in any one of SEQ ID NOs: 139-159. In some cases, an IRES sequence comprises a nucleotide sequence having 95% or more (e.g., 97% or more, 98% or more, 99% or more, or 100% sequence identity) with the nucleotide sequence set forth in any one of SEQ ID NOs: 139-159. In some cases, an IRES sequence comprises a nucleotide sequence comprising the nucleotide sequence set forth in any one of SEQ ID NOs: 139-159.

In some cases, an IRES sequence is selected from the group consisting of the following IRES sequences: EMCV, BIP, CAT-1, c-myc, HCV, VCIP, Apaf-1, mEMCV-1, mEMCV-2, HRV, NRF, FGF-1, KMI1, KM12, (GAAA)16, (PPT19)4, EMCV mutant 5 (see FIG. 13A “mutant 5”), EMCV mutant 10 (see FIG. 13A “mutant 10”), EMCV mutant 15 (see FIG. 13A “mutant 15”), and EMCV mutant 21 (see FIG. 13A “mutant 21”).

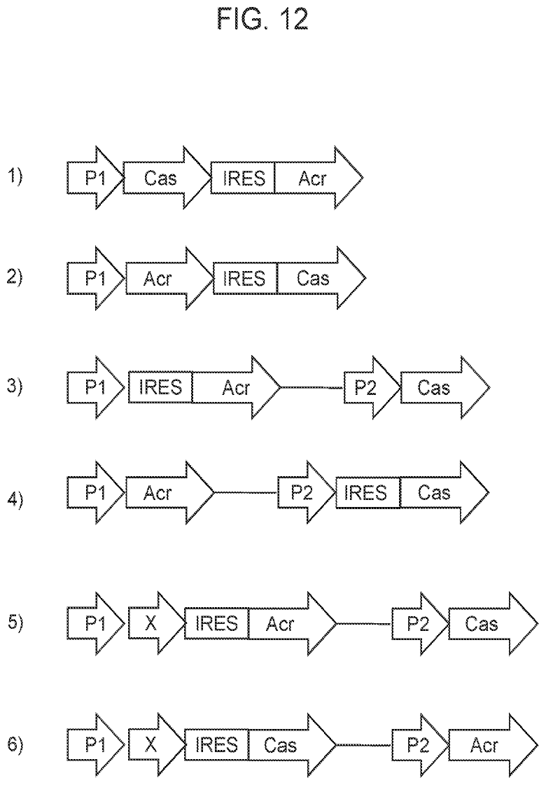

Typically, a subject IRES sequence will be positioned in a subject nucleic acid so as to regulate the expression (translation) of a subject protein (e.g., Acr protein or Cas protein)—and as such will be positioned 5′ of (usually immediately 5′ of) the protein coding sequence which it regulates. FIG. 12 provides non-limiting illustrative examples of embodiments in which and IRES sequence is positioned in different ways. In some cases, an IRES sequence is positioned 5′ of (and usually immediately 5′ of) a Cas protein coding sequence. In some cases, an IRES sequence is positioned 5′ of (and usually immediately 5′ of) an Acr protein coding sequence.

In some cases, the Cas-encoding sequence and the Acr-encoding sequence are operably linked to the same promoter (encoded by a polycistronic sequence) and are positioned in tandem, with an IRES sequence positioned between them (see, e.g., FIG. 12, first and second examples). In some such cases, the Cas protein encoding sequence is positioned 5′ of the Acr encoding sequence, and the IRES sequence is therefore 3′ of the Cas sequence and 5′ of the Acr sequence. In some such cases, the Acr protein encoding sequence is positioned 5′ of the Cas encoding sequence, and the IRES sequence is therefore 3′ of the Acr sequence and 5′ of the Cas sequence.

In some cases, the Cas-encoding sequence and the Acr-encoding sequence are operably linked to a first promoter and a second promoter, respectively, such that they are transcribed as separate transcripts (see, e.g., FIG. 12, third through sixth examples). The first and second promoters (labeled “P1” and “P2” in the figure) can be different from one another or can be the same (i.e., can be a copy of the same promoter). In some such cases, the IRES sequence regulates (and is therefore positioned 5′ of) the Acr sequence. In other cases, the IRES sequence regulates (and is therefore positioned 5′ of) the Cas sequence. In some cases, in which the Cas-encoding sequence and the Acr-encoding sequence are transcribed as separate sequences, each one is regulated by an IRES sequence (and each is therefore positioned 3′ of an IRES sequence).

In some embodiments in which the Cas-encoding sequence and the Acr-encoding sequence are transcribed as separate sequences, a “spacer” protein coding sequence is used 5′ of the IRES sequence such that the ‘spacer’ sequence is transcribed as part of a polycistronic sequence with the protein sequence being regulated (see, e.g., the fifth and sixth examples of FIG. 12). For example, in the fifth example of FIG. 12, a Cas protein coding sequence and an Acr protein coding sequence are operably linked to different promoters (P1 and P2). A spacer sequence (labeled as “X” in the figure) is positioned 5′ of an IRES sequence, which is 5′ of an Acr coding sequence—and therefore the spacer sequence and the Acr sequence are transcribed as part of the same RNA, but the presence of the IRES sequence causes the Acr protein to be produced as a separate protein. Likewise, in the sixth example of FIG. 12, a Cas protein coding sequence and an Acr protein coding sequence are again operably linked to different promoters (P1 and P2). In this example, a spacer sequence (labeled as “X” in the figure) is positioned 5′ of an IRES sequence, which is 5′ of a Cas coding sequence—and therefore the spacer sequence and the Cas sequence are transcribed as part of the same RNA, but the presence of the IRES sequence causes the Cas protein to be produced as a separate protein.

A ‘spacer’ protein can be any desired sequence—as its purpose is to simply provide a sequence to be translated that is 5′ of the IRES sequence. A spacer sequence can be any convenient length, from very short to encoding an entire protein sequence. In some cases, the spacer is 2 or more amino acids long (e.g., 3 or more, 4 or more 5 or more, 10 or more, or 20 or more amino acids). In some cases, the spacer has a length of from 1 to 100 amino acids (e.g., 1 to 80, 1 to 50, 1 to 40, 1 to 30, 1 to 20, 1 to 10, 2 to 100, 2 to 80, 2 to 50, 2 to 40, 2 to 30, 2 to 20, 2 to 10, 5 to 100, 5 to 80, 5 to 50, 5 to 40, 5 to 30, 5 to 20, or 5 to 10 amino acids). Examples of spacer sequence include, but are not limited to: linker sequences, repeated single amino acids (e.g., AAAA), random sequences, fragments of proteins, and marker proteins (e.g., a fluorescent protein such as GFP, YFP, CFP, RFP, and the like, a drug selectable protein marker, an enzyme such as beta-galactosidase, etc.).

In some cases, in which the Acr encoding sequence and the Cas encoding sequences are operably linked to separate promoters, a spacer sequence is not used (see, e.g., the third and fourth examples of FIG. 12).

Start Codon

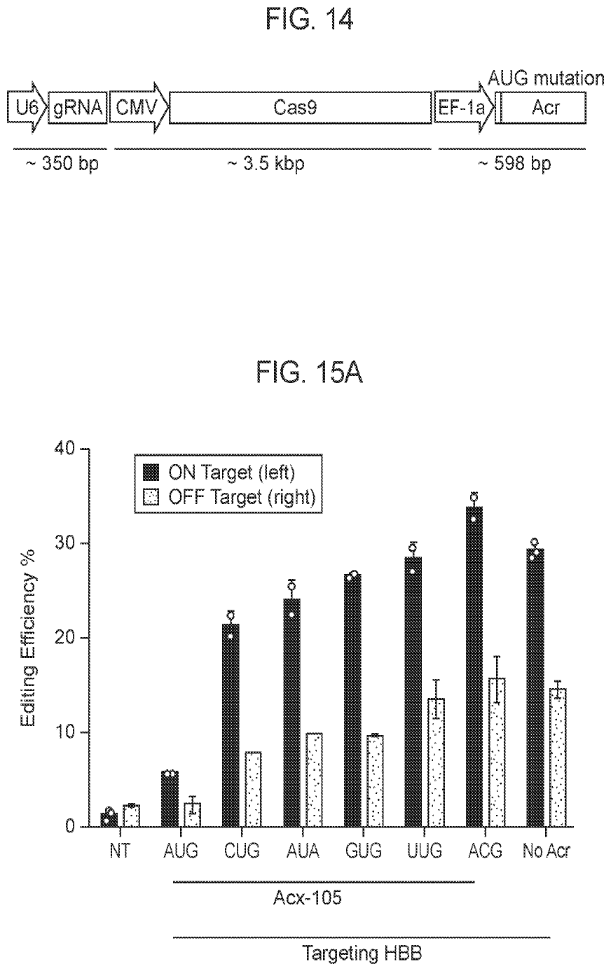

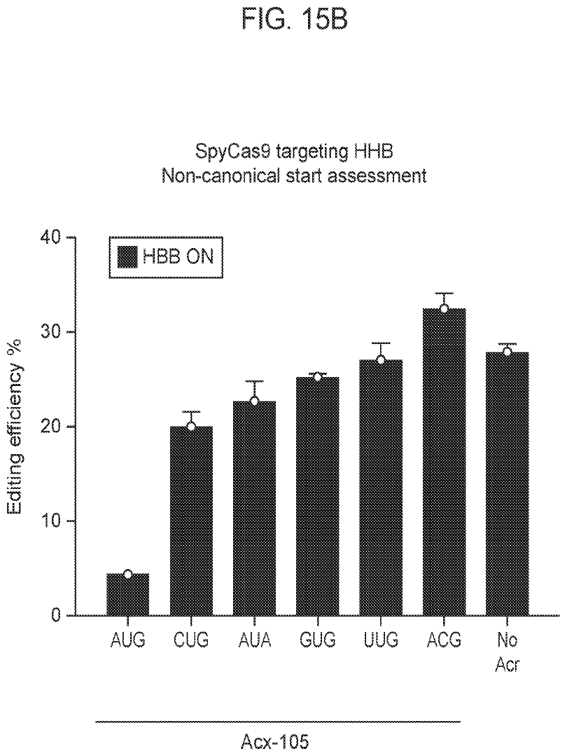

One non-limiting example of a translational control element is a non-AUG start codon (also referred to as a non-AUG initiation codon). The term “non-AUG start codon” or “non-AUG initiation codon” is meant to include any non-AUG polynucleotide (typically a triplet) that functions as a start site for translation initiation with reduced efficiency relative to that of an AUG start codon. Examples of naturally occurring alternate start codon usage are described for example in Kozak (1991) J. Cell Biol. 115(4): 887-903; Mehdi et al. (1990) Gene 91:173-178; Kozak (1989) Mol. Cell. Biol. 9(11): 5073-5080. In general, non-AUG start codons have decreased translation efficiencies compared to that of an AUG; for example, the alternate start codons CUG, GUG, ACG, AUA, and UUG were tested in the working examples below (see, e.g., FIG. 14, FIG. 15, Table 8c and Table 9c) and all exhibited decreased translation relative to AUG.

In some cases, a non-AUG start codon is used as the initiation codon for the sequence encoding the Acr protein. In some cases, a non-AUG start codon is used as the initiation codon for the sequence encoding the Cas protein (e.g., Cas effector protein). In some cases, a non-AUG start codon (used with the Acr sequence or with the Cas sequence) is any one of: CUG, GUG, ACG, AUA, UUG, GCG, AGO. AAG, AUC. or AUU. In some cases, a non-AUG start codon (used with the Acr sequence or with the Cas sequence) is any one of: CUG, GUG, ACG, AUA, or UUG. For example, in some cases a non-AUG start codon used with the Acr sequence is any one of: CUG, GUG, ACG, AUA, UUG, GCG, AGG, AAG, AUC, or AUU. In some cases, a non-AUG start codon used with the Acr sequence is any one of: CUG, GUG, ACG, AUA, or UUG. As another example, in some cases a non-AUG start codon used with the Cas sequence is any one of: CUG, GUG, ACG, AUA, UUG, GCG, AGG, AAG, AUC, or AUU. In some cases, a non-AUG start codon used with the Cas sequence is any one of: CUG, GUG, ACG, AUA, or UUG. In some cases, a non-AUG start codon used with the Acr sequence is CUG. In some cases, a non-AUG start codon used with the Acr sequence is GUG. In some cases, a non-AUG start codon used with the Acr sequence is ACG. In some cases, a non-AUG start codon used with the Cas sequence is CUG. In some cases, a non-AUG start codon used with the Cas sequence is GUG. In some cases, a non-AUG start codon used with the Cas sequence is ACG.

The translation efficiency of a non-AUG start codon can also be affected by its sequence context; for example, in eukaryotic cells an optimal Kozak consensus sequence has been reported to have a positive effect on translation initiation at non-AUG start codons (Mehdi et al. (1990) Gene 91:173-178; Kozak (1989) Mol. Cell. Biol. 9(11): 5073-5080). The complete Kozak DNA consensus sequence is GCCRCCATGG (SEQ ID NO:160), where the start codon ATG (AUG in RNA) is just prior to the final “G”, the A of the ATG start codon is designated as the +1 position, and “R” at position −3 is a purine (A or G). The two most highly conserved positions are a purine, usually an A, at −3 and a G at +4 (Kozak (1991) J Cell Biol 115(4): 887-903). In some cases, a subject non-AUG start codon (e.g., any of those discussed above) is coupled with an impaired Kozak sequence (i.e., a Kozak sequence that does not conform to the consensus.

For examples of the above, see, e.g., Kearse and Wilusz, Genes Dev. 2017 Sep. 1; 31(17):1717-1731; U.S. Patent Application Publication Nos. US20060172382 and US20060141577; and U.S. Pat. Nos. 5,648,267; 5,733,779; 8,828,976; 10,030,252; 10,317,329; the disclosures of which, as they relate to Kozak sequences and non-AUG start codons (as well as assays associated therewith), are incorporated herein by reference. One of skill in the art will recognize that the sequences described herein as DNA will have correlative sequences as RNA molecules, e.g., DNA sequence ATG would correspond to RNA sequence AUG, and vice versa.

Typically, a subject non-AUG initiation codon will be positioned in a subject nucleic acid so as to regulate the expression (translation initiation) of a subject protein (e.g., Acr protein or Cas protein)—and as such will be positioned 5′ of (usually immediately 5′ of) and in frame with the protein coding sequence which it regulates. In some cases (e.g., when a non-AUG start codon is used as the initiation codon for the Acr encoding sequence), the sequence encoding the Acr protein does not include its native AUG start codon. In some such cases, the sequence encoding the Acr protein does not include an AUG codon. In some cases (e.g., when a non-AUG start codon is used as the initiation codon for the Cas encoding sequence), the sequence encoding the Cas protein (e.g., Cas effector protein) does not include its native AUG start codon. In some cases, the sequence encoding the Cas protein (e.g., Cas effector protein) does not include an AUG codon. In some cases, (e.g., when a non-AUG start codon is used as the initiation codon for the Cas-encoding or Acr-encoding sequence), the sequence encoding the subject protein, i.e., the Cas protein (e.g., Cas effector protein) or the Acr protein, is codon optimized as a whole or in part, to avoid having an out-of-frame AUG that could direct translational machinery to an incorrect reading frame contained in the nucleic acid encoding the Cas or Acr protein (i.e., a reading frame that does not encode the subject protein). In some such cases, the first 10, 15, 20, 25, 30, 35, 40, 45, 50 or more than 50 codons from the start of the subject protein are optimized so as not to include an out-of-frame AUG.

In some cases, the Cas-encoding sequence and the Acr-encoding sequence are operably linked to a first promoter and a second promoter, respectively, such that they are transcribed as separate transcripts (see, e.g., FIG. 18). The first and second promoters (labeled “P1” and “P2” in the figure) can be different from one another or can be the same (i.e., can be a copy of the same promoter).

In some embodiments, more than one translational control element can be used to control expression of a protein (e.g., Acr. Cas effector protein). For example, in some cases more than one (e.g., two, two or more, three) translational control elements are used to control expression of the Acr protein. In some cases, more than one (e.g., two, two or more, three) translational control elements are used to control expression of the Cas effector protein. As such, in some cases one or more (e.g., two, two or more, three) translational control elements (e.g., 2A peptide. IRES, non-AUG start codon) are used to control expression of the Acr protein and/or the Cas effector protein. In some cases, one or more (e.g., two, two or more, three) translational control elements (e.g., 2A peptide, IRES, non-AUG start codon) are used to control expression of the Acr protein. In some cases, one or more (e.g., two, two or more, three) translational control elements (e.g., 2A peptide. IRES, non-AUG start codon) are used to control expression of the Cas effector protein.

In some cases, a combination of translational control elements is used to control expression of both the Acr protein and the Cas effector protein. For example, in some cases a non-AUG start codon is used to control translation from a polycistronic transcript that includes a 2A peptide coding sequence separating the Acr protein and Cas effector protein coding sequences. For example, in some cases the following are present, in order from 5′ to 3′ prime: a non-AUG start codon, a sequence encoding an Acr protein, a 2A peptide coding sequence, a Cas effector protein coding sequence. In some cases the following are present, in order from 5′ to 3′ prime: a non-AUG start codon, a sequence encoding a Cas effector protein, a 2A peptide coding sequence, an Acr protein coding sequence.

Likewise, in some cases a non-AUG start codon is used to control translation from a polycistronic transcript that includes an IRES sequence, such that the non-AUG start codon can be used to control expression of a first protein (e.g., Acr protein, Cas effector protein), and an IRES can be present on the same transcript to control expression of a second protein (e.g., Acr protein, Cas effector protein). For example, in some cases the following are present, in order from 5′ to 3′ prime: a non-AUG start codon, a sequence encoding an Acr protein, an IRES sequence, a Cas effector protein coding sequence. In some cases, the following are present, in order from 5′ to 3′ prime: a non-AUG start codon, a sequence encoding a Cas effector protein, an IRES sequence, an Acr protein coding sequence.

In some cases, a 2A peptide sequence and an IRES sequence can be used in combination. For example, an Acr protein or a Cas effector protein can be separated from a third protein coding sequence using a 2A peptide sequence, while the Acr protein and Cas effector protein are separated from one another by an IRES sequence. As an example, the Acr protein coding sequence can be separated from a third protein coding sequence using a 2A peptide sequence, while an IRES sequence separates those coding sequences from the Cas effector protein coding sequence. Likewise, the Cas effector protein coding sequence can be separated from a third protein coding sequence using a 2A peptide sequence, while an IRES sequence separates those coding sequences from the Acr protein coding sequence. And in any of the above scenarios, a non-AUG start codon can be used to control the translation of the proteins that are separated by a 2A peptide sequence. Moreover, a 2A peptide sequence can be used between two protein coding sequences that both follow an IRES sequence. For example, both an Acr protein coding sequence and a Cas effector protein coding sequence could follow an IRES sequence, and a 2A peptide sequence can be used to separate the Acr protein coding sequence from the Cas effector protein coding sequence.

Thus, any convenient combination of translational control elements can be used.

The following are illustrative examples of using multiple translational control elements:
a 2A peptide and an IRES sequence (e.g.: protein 1—2A—protein 2—IRES—protein 3; protein 1—IRES—protein 2—2A—protein 3) [where the Acr protein and the Cas effector protein can each be protein 1, 2, or 3]
a 2A peptide and a non-AUG start codon (e.g., non-AUG—protein 1—2A—protein 2) [where the Acr protein and the Cas effector protein can each be protein 1 or 2]
an IRES sequence and a non-AUG start codon (e.g., non-AUG—protein 1—IRES—protein 2) [where the Acr protein and the Cas effector protein can each be protein 1 or 2]
a 2A peptide, an IRES sequence, and a non-AUG start codon (e.g., non-AUG—protein 1—2A—protein 2—IRES protein 3; non-AUG—protein 1—IRES—protein 2—2A—protein 3) [where the Acr protein and the Cas effector protein can each be protein 1, 2, or 3]

### Promoters

The present disclosure provides coordinated delivery systems (and methods of using such systems) that includes delivery of expression cassettes, such as on one or more vectors, and which expression cassettes include promoters to drive expression of the genes encoding a Cas protein (e.g., a Class 2 effector protein) and an Acr protein. In one embodiment, the vector includes a first expression cassette that includes a first promoter operably linked to a sequence encoding an anti-CRISPR (Acr) protein, and a second expression cassette that includes a second promoter operably linked to a sequence encoding a CRISPR-associated (Cas) protein.

In some cases, an Acr coding sequence and a Cas effector protein sequence are operably linked to the same promoter and are therefore transcribed as part of the same RNA. In other cases, an Acr coding sequence and a Cas effector protein sequence are operably linked to different promoters. For example, in some cases an Acr coding sequence is operably linked to a first promoter and a Cas effector protein sequence is operably linked to a second promoter. In some such cases the first and second promoters are the same—such that the two protein coding sequences are transcribed as separate RNAs, but are controlled by the same promoter sequence (i.e., there are two copies of the same promoter—one controlling expression of one protein and another controlling expression of the other). In other such cases the first and second promoters are different promoters.

### Promoter Types

The coordinated delivery systems described herein encompass a variety of promoter types that can be used, e.g., to control the expression of an Acr protein and/or Cas effector protein (e.g., Class 2 effector nuclease). A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any convenient organism. Suitable promoters can be derived from viruses and can therefore be referred to as viral promoters, or they can be derived from any organism, including prokaryotic or eukaryotic organisms. Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like. Pol III promoters such as U6, enhanced U6, and H1, are generally used to express non-coding RNAs such as guide RNAs.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a subject site-directed modifying polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIIα) promoter (see, e.g., Mayford et al. (1996) Proc. Natl. Acad. Sci. USA 93:13250; and Casanova et al. (2001) Genesis 31:37); a CMV enhancer/platelet-derived growth factor-β promoter (see, e.g., Liu et al. (2004) Gene Therapy 11:52-60); and the like.

Suitable liver-specific promoters can in some cases include, but are not limited to: TTR, Albumin, and AAT promoters. Suitable CNS-specific promoters can in some cases include, but are not limited to: Synapsin 1, BM88, CHNRB2, GFAP, and CAMK2a promoters. Suitable muscle-specific promoters can in some cases include, but are not limited to: MYOD1, MYLK2, SPc5-12 (synthetic), a-MHC, MLC-2, MCK, MHCK7, human cardiac troponin C (cTnC) and desmin promoters.

Adipocyte-specific spatially restricted promoters include, but are not limited to, aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, α-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to, an SM22α promoter (see, e.g., Akyurek et al. (2000) Mol. Med. 6:983; and U.S. Pat. No. 7,169,874); a smoothelin promoter (see, e.g., WO 2001/018048); an α-smooth muscle actin promoter; and the like. For example, a 0.4 kb region of the SM22α promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

In some cases, a subject vector includes an enhancer sequence. Suitable enhancers include but are not limited to ApoE and HBV EII.

Examples of inducible promoters include, but are not limited to, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; an estrogen receptor; an estrogen receptor fusion; an estrogen analog; IPTG; etc.

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; an estrogen analog; IPTG; etc

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Inducible promoters include sugar-inducible promoters (e.g., lactose-inducible promoters; arabinose-inducible promoters); amino acid-inducible promoters; alcohol-inducible promoters; and the like. Suitable promoters include, e.g., lactose-regulated systems (e.g., lactose operon systems, sugar-regulated systems, isopropyl-beta-D-thiogalactopyranoside (IPTG) inducible systems, arabinose regulated systems (e.g., arabinose operon systems, e.g., an ARA operon promoter, pBAD, pARA, portions thereof, combinations thereof and the like), synthetic amino acid regulated systems, fructose repressors, a tac promoter/operator (pTac), tryptophan promoters, PhoA promoters, recA promoters, proU promoters, cst-1 promoters, tetA promoters, cadA promoters, nar promoters, PL promoters, cspA promoters, and the like, or combinations thereof. In certain cases, a promoter comprises a Lac-Z, or portions thereof. In some cases, a promoter comprises a Lac operon, or portions thereof. In some cases, an inducible promoter comprises an ARA operon promoter, or portions thereof. In certain embodiments an inducible promoter comprises an arabinose promoter or portions thereof. An arabinose promoter can be obtained from any suitable bacteria. In some cases, an inducible promoter comprises an arabinose operon of E. coli or B. subtilis. In some cases, an inducible promoter is activated by the presence of a sugar or an analog thereof. Non-limiting examples of sugars and sugar analogs include lactose, arabinose (e.g., L-arabinose), glucose, sucrose, fructose, IPTG, and the like. Suitable promoters include a T7 promoter; a pBAD promoter; a lacIQ promoter; and the like. In some cases, the promoter is a J23119 promoter. Many bacterial promoters are known in the art; bacterial promoters can be found on the internet at parts(dot)igem (dot)org/promoters.

In some cases, the promoter is a reversible promoter. Suitable reversible promoters, including reversible inducible promoters are known in the art. Such reversible promoters may be isolated and derived from many organisms. Such reversible promoters may be isolated and derived from many organisms, e.g., eukaryotes and prokaryotes. Modification of reversible promoters derived from a first organism for use in a second organism is well known in the art. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Tables 4-6 (including 4a and 4b) provide examples of promoters functional in various cell types, including bacterial, insect, plant, and mammalian cells.

TABLE 4a
Examples of bacterial promoters

| Promoter | Expression | Description |
|---|---|---|
| T7 | Constitutive but requires T7 RNA | Promoter from T7 |
| | polymerase (e.g., inducible by | bacteriophage |
| | presence of T7 RNA polymerase) | |
| Sp6 | Constitutive but requires Sp6 RNA | Promoter from Sp6 |
| | polymerase (e.g., inducible by | bacteriophage |
| | presence of Sp6 RNA polymerase) | |
| lac | Constitutive in the absense of lac | Promoter from Lac |
| | repressor (lacl or laclq). Can be | operon |
| | induced by IPTG or lactose | |
| araBad | Inducible by arabinose | Promoter of the |
| | | arabinose metabolic |
| | | operon |
| trp | Repressible by tryptophan | Promoter from E. coli |
| | | tryptophan operon |
| Ptac | Regulated like the lac promoter | Hybrid promoter of |
| | | lac and trp |

TABLE 4b
Examples of plant promoters

| Constitutive plant origin | Ref |
|---|---|
| Arabidopsis | An et al., 1966 |
| Maize pUbi1 | Christensen et al., 1992 |
| Nicotiana sylvestris Ubi. U4 | Plesse et al., 2001 |
| Inducible Promoters | |
| In2-2 | De Veylder et al., 1997 |
| Copper inducible | Mett et al., 1993 |
| Alc system | Caddick et al., 1998 |
| DEX system | Aoyama and Chua., 1997 |
| Tet/DEX system | Bohner et al., 1999 |
| potato wun1 | Siebertz et al., 1989 |
| Floral-specific | |
| Chrysanthemum UEP1 | Annadana et al., 2002 |
| Bean CHS15 | Factor et al., 1996 |
| Petunia EPSPS | Benfey et al., 1990 |
| Constitutive viral origin | |
| CaMV 35S | Odell et al., 1985 |
| Pollen Specific | |
| Maize ZMC5 | Wakeley et al., 1998 |
| Tomato lat52 | Twell et al., 1989 |
| Pistil specific | |
| Pear PsTL1 | Sassa et al., 2002 |
| Potato SK2 | Ficker et al., 1997 |
| Anther specific | |
| Tobacco TA29 | Koltunow et al., 1990 |
| Rice RA8 | Jeon et al., 1999 |
| Green tissue specific | |
| Pea rbcS-3A | Gilmartin and Chua, 1990 |
| Arabidposis CAB2 | Carre de Kay, 1995 |
| Alfalfa RAc | Potenza et al., unpublished |
| Organelle specific | |
| PsbA | Satub and Maliga, 1994 |
| RbcL | Shiina et al., 1998 |
| Prrn | Maliga, 2002 |
| Fruit specific | |
| Apple ACC oxidase | Atkinson et al., 1998 |
| Tomato polygalacturonase | Fraser et al., 2002 |
| Tomato E8 | Deikman and Fischer, 1988 |
| Tomato Pds | Corona et al., 1990 |
| Nodule specific | |
| Vicia faba VtEnod12 | Fruhling et al., 2000 |
| Bean Nv30 | Carsolio et al., 1994Szabados |
| | et al., 1990 |
| S. rostrata leghemoglobin | |
| Seed coat specific | |
| Pea PsGNS2 | Buchner et al., 2002 |
| Seed Specific | |
| Bean beta-phaseolin | Bustos et al., 1989 |
| Cotton alpha-globulin | Sunilkumar et al., 2002 |
| Wheat gbss1 | Kluth et al., 2002 |

TABLE 5
Examples of Insect promoters

| Promoter | Notes |
|---|---|
| OpIE2 | *From the Orgyia pseudotsugata multicapsid |
| | nucleopolyhedrosis virus (for high level expression). |
| | *Active in many common insect cell types including |
| | SF9, SF21, High Five ™, Mimic-SF9, S2, MG1and |
| | KC1 cells |
| PvGapdh | constitutive promoter for heterologous gene |
| | expression in Pv11 cells |
| Promoter 121 | |
| Drosophila MT | |
| promoter | |
| AcMNPV | can be linked to a AcMNPV enhancer element |
| immediate early | (hr5) for stronger expression |
| (ie1) | |
| AcMNPV | can be linked to a AcMNPV enhancer element |
| delayed early | (hr5) for stronger expression |
| (39K) | |
| AcMNPV late | can be linked to a AcMNPV enhancer element |
| (p6.9) | (hr5) for stronger expression |
| AcMNPV very | can be linked to a AcMNPV enhancer element |
| late (polh) | (hr5) for stronger expression |

TABLE 6
Examples of constitutive mammalian promoters
and their strength of expression

| Name | Description | Strength |
|---|---|---|
| CMV/miniCMV | Human cytomegalovirus immediate early | Strong |
| | enhancer/promoter | |
| EF1A | Human eukaryotic translation elongation | Strong |
| | factor 1 α1 promoter | |
| EFS | Human eukaryotic translation elongation | Medium |
| | factor 1 α1 short form | |
| CAG | CMV early enhancer fused to modified | Strong |
| | chicken β-actin promoter | |
| CBh | CMV early enhancer fused to modified | Strong |
| | chicken β-actin promoter | |
| SV40 | Simian virus 40 enhancer/early promoter | Medium |
| hPGK | Human phosphoglycerate kinase 1 promoter | Medium |
| UBC | Human ubiquitin C promoter | Weak |

Suitable promoters include but are not limited to the following:

### Mammalian (Pol II) Promoters (for Nuclease and Acrs)

retroviral Rous sarcoma virus (RSV)
LTR promoter (optionally with the RSV enhancer),
cytomegalovirus (CMV) promoter (optionally with the CMV enhancer)
SV40 promoter
dihydrofolate reductase promoter,
beta.-actin promoter,
phosphoglycerol kinase (PGK) promoter,
EF1.alpha. (EF1a) promoter.
MMLV LTR promoters
HIV LTR promoters, MCMV LTR promoters,
MND,
Ubc,
CAG,
HSV TK promoter,
fos promoter,
E2F promoter
polyoma virus
adenovirus, fowlpox virus
bovine papilloma virus
avian sarcoma virus

### Eukaryotic Tissue-Specific

Bowman et al., 1995 Proc. Natl. Acad. Sci. USA 92, 12115-12119 describe a brain-specific transferrin promoter;
synapsin I promoter is neuron specific (Schoch et al., 1996 J. Biol. Chem. 271, 3317-3323);
necdin promoter is post-mitotic neuron specific (Uetsuki et al., 1996 J. Biol. Chem. 271, 918-924);
neurofilament light promoter is neuron specific (Charron et al., 1995 J. Biol. Chem. 270, 30604-30610);
acetylcholine receptor promoter is neuron specific (Wood et al., 1995 J. Biol. Chem. 270, 30933-30940);
potassium channel promoter is high-frequency firing neuron specific (Gan et al., 1996 J. Biol. Chem 271, 5859-5865);
chromogranin A promoter is neuroendocrine cell specific (Wu et al., 1995 A. J. Clin. Invest. 96, 568-578);
Von Willebrand factor promoter is brain endothelium specific (Aird et al., 1995 Proc. Natl. Acad. Sci. USA 92, 4567-4571);
flt-1 promoter is endothelium specific (Morishita et al., 1995 J. Biol. Chem. 270, 27948-27953);
preproendothelin-1 promoter is endothelium, epithelium and muscle specific (Harats et al., 1995 J. Clin. Invest. 95, 1335-1344);
GLUT4 promoter is skeletal muscle specific (Olson and Pessin, 1995 J. Biol. Chem. 270, 23491-23495);
Slow/fast troponins promoter is slow/fast twitch myofibre specific (Corin et al., 1995 Proc. Natl. Acad. Sci. USA 92, 6185-6189);
Actin promoter is smooth muscle specific (Shimizu et al., 1995 J. Biol. Chem. 270, 7631-7643);
Myosin heavy chain promoter is smooth muscle specific (Kallmeier et al., 1995 J. Biol. Chem. 270, 30949-30957);
E-cadherin promoter is epithelium specific (Hennig et al., 1996 J. Biol. Chem. 271, 595-602);
cytokeratins promoter is keratinocyte specific (Alexander et al., 1995 B. Hum. Mol. Genet. 4, 993-999);
transglutaminase 3 promoter is keratinocyte specific (J. Lee et al., 1996 J. Biol. Chem. 271, 4561-4568);
bullous pemphigoid antigen promoter is basal keratinocyte specific (Tamai et al., 1995 J. Biol. Chem. 270, 7609-7614);
keratin 6 promoter is proliferating epidermis specific (Ramirez et al., 1995 Proc. Natl. Acad. Sci. USA 92, 4783-4787);
collagen 1 promoter is hepatic stellate cell and skin/tendon fibroblast specific (Houglum et al., 1995 J. Clin. Invest. 96, 2269-2276);
type X collagen promoter is hypertrophic chondrocyte specific (Long & Linsenmayer, 1995 Hum. Gene Ther. 6, 419-428);
Factor VII promoter is liver specific (Greenberg et al., 1995 Proc. Natl. Acad. Sci. USA 92, 12347-1235);
fatty acid synthase promoter is liver and adipose tissue specific (Soncini et al., 1995 J. Biol. Chem. 270, 30339-3034);
carbamoyl phosphate synthetase I promoter is portal vein hepatocyte and small intestine specific (Christoffels et al., 1995 J. Biol. Chem. 270, 24932-24940); the Na-K-CI transporter promoter is kidney (loop of Henle) specific (Igarashi et al., 1996 J. Biol. Chem. 271, 9666-9674);
scavenger receptor A promoter is macrophages and foam cell specific (Horvai et al., 1995 Proc. Natl. Acad. Sci. USA 92, 5391-5395);
glycoprotein IIb promoter is megakaryocyte and platelet specific (Block & Poncz, 1995 Stem Cells 13, 135-145);
yc chain promoter is hematopoietic cell specific (Markiewicz et al., 1996 J. Biol. Chem. 271, 14849-14855); CD11b promoter is mature myeloid cell specific (Dziennis et al., 1995 Blood 85, 31 9-329).

### Yeast Promoters

TRP1
ADHI
ADHII
acid phosphatase (PH05)
enolase,
glyceraldehyde-3-phosphate dehydrogenase (GAP),
3-phospho glycerate kinase (PGK),
hexokinase,
pyruvate decarboxylase,
phosphofructokinase,
glucose-6-phosphate isomerase,
3-phosphoglycerate mutase
pyruvate kinase
triose phosphate isomerase
phosphoglucose isomerase
glucokinase
GAL 4
S. pombe nmt 1
TATA binding protein (TBP)

### Inducible for Yeast

alcohol dehydrogenase 2,
isocytochrome C
acid phosphatase,
metallothionein,
GAP

### Yeast Including Pichia

YHR140W, YNL040W, NTA1, SGT1, URK1, PGI1, YHR112C, CPS1, PET18, TPA1, PFK1, SCS7, YIL166C, PFK2, HSP12, ERO1, ERG11, ENO1, SSP120, BNA1, DUG3, CYS4, YEL047C, CDC19, BNA2, TDH3, ERG28, TSA1, LCB5, PLB3, MUP3, ERV14, PDX3, NCP1, TPO4, CUS1, COX15, YBR096W, DOG1, YDL124W, YMR244W, YNL134C, YEL023C, PIC2, GLK1, ALD5, YPRO98C, ERG1, HEM13, YNL200C, DBP3, HAC1, UGA2, PGK1, YBRO56W, GEF1, MTD1, PDR16, HXT6, AQR1, YPL225W, CYS3, GPM1, THI11, UBA4, EXG1, DGK1, HEM14, SCO1, MAK3, ZRT1, YPL260W, RSB1, AIM19, YET3, YCR061W, EHT1, BAT1, YLR126C, MAE1, PGC1, YHLOO8C, NCE103, MIH1, ROD1, FBA1, SSA4, PIL1, PDC1-3, TH13, SAM2, EFT2, and INO1.

### Insect Promoters

synthetic disclosed in US 20100167389
Insect minimal promoters disclosed in US20070056051 (alone or in combination with enhancers)
   mini-white (white promoter):
   Act5C promoter
   ubi-p63E promoter
   BmA3 promoter
   hr enhancer and ie1 promoter
polyhedrin promoter (U.S. Pat. No. 4,745,051; Vasuvedan et al., 1992, FEBS Lett. 311: 7-11),
P10 promoter (Vlak et al., 1988, J. Gen. Virol. 69: 765-776),
Autographa californica polyhedrosis virus basic protein promoter (EP 397485),
baculovirus immediate-early gene promoter gene 1 promoter (U.S. Pat. Nos. 5,155,037 and 5,162,222)
baculovirus 39K delayed-early gene promoter (also U.S. Pat. Nos. 5,155,037 and 5,162,222)
OpMNPV immediate early promoter 2;

### Plant and Algae Promoters

pLGV23,
pGHlac+,
pBIN19,
pAK2004,
pVKH
pDH51 (for above see Schmidt, R. and Willmitzer, L., Plant Cell Rep. 7, 583 (1988)).
constitutive expression (Benfey et al., EMBO J. 8, 2195 (1989))
   35S CaMV (Franck et al., Cell 21, 285 (1980)),
   19S CaMV (see also U.S. Pat. No. 5,352,605 and PCT Application No. WO 84/02913)
   Rubisco small subunit described in U.S. Pat. No. 4,962,028.
   uper-promoter (Ni et al., Plant Journal 7, 661 (1995)),
   Ubiquitin promoter (Callis et al., J. Biol. Chem., 265, 12486 (1990); U.S. Pat. Nos. 5,510,474; 6,020,190; Kawalleck et al., Plant. Molecular Biology, 21, 673 (1993))
   34S promoter (GenBank Accession numbers M59930 and X16673)
Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters, and the like. Seed preferred promoters are preferentially expressed during seed development and/or germination. For example, seed preferred promoters can be embryo-preferred, endosperm preferred, and seed coat-preferred. See Thompson et al., BioEssays 10, 108 (1989).
seed preferred promoters
   cellulose synthase (ceIA),
   Cim1,
   gamma-zein,
   globulin-1,
   maize 19 kD zein (cZ19B1)
   U.S. Pat. No. 5,608,152 (napin promoter from rapeseed),
   WO 98/45461 (phaseolin promoter from Arabidopsis),
   U.S. Pat. No. 5,504,200 (phaseolin promoter from Phaseolus vulgaris),
   WO 91/13980 (Bce4 promoter from Brassica)
   Baeumlein et al., Plant J., 2 (2), 233 (1992) (LEB4 promoter from leguminosa).
   Ipt-2- or Ipt-1-promoter from barley (WO 95/15389 and WO 95/23230)
   hordein promoter from barley.
Other promoters
   major chlorophyll a/b binding protein promoter,
   histone promoters,
   Ap3 promoter,
   beta.-conglycin promoter,
   napin promoter,
   soybean lectin promoter,
   maize 15 kD zein promoter,
   22 kD zein promoter
   27 kD zein promoter
   g-zein promoter,
   waxy,
   shrunken 1,
   shrunken 2
   bronze promoter
   Zm13 promoter (U.S. Pat. No. 5,086,169)
   maize polygalacturonase promoters (PG) (U.S. Pat. Nos. 5,412,085 and 5,545,546)
   SGB6 promoter (U.S. Pat. No. 5,470,359),
   PRP1 (Ward et al., Plant. Mol. Biol. 22, 361 (1993))
   SSU,
   OCS,
   lib4,
   usp,
   STLS1
   B33,
   LEB4,
   nos,
   ubiquitin,
   napin
   phaseolin
   cytoplasmic FBPase promotor
   ST-LSI promoter of potato (Stockhaus et al., EMBO J. 8, 2445 (1989)),
   phosphorybosyl phyrophoshate amido transferase promoter of Glycine max (gene bank accession No. U87999)
   noden specific promoter described in EP-A-0 249 676
inducible promoters
   EP 388 186 (benzyl sulfonamide inducible),
   Gatz et al., Plant J. 2, 397 (1992) (tetracyclin inducible), EP-A-0 335 528 (abscisic acid inducible)
   WO 93/21334 (ethanol or cyclohexenol inducible)
   auxin-response elements E1 promoter fragment (AuxREs) in the soybean (Glycine max L.) (Liu (1997) Plant Physiol. 115:397-407);
   auxin-responsive Arabidopsis GST6 promoter (also responsive to salicylic acid and hydrogen peroxide) (Chen (1996) Plant J. 10: 955-966);
   auxin-inducible parC promoter from tobacco (Sakai (1996) 37:906-913)
   plant biotin response element (Streit (1997) Mol. Plant Microbe Interact. 10:933-937);
   promoter responsive to the stress hormone abscisic acid (Sheen (1996) Science 274:1900-1902).
drought-specific promoter
   maize rab17 drought-inducible promoter (Vilardell et al. (1991) Plant Mol. Biol. 17:985-993; Vilardell et al. (1994) Plant Mol. Biol. 24:561-569));
   cold, drought, and high salt inducible promoter from potato (Kirch (1997) Plant Mol. Biol. 33:897-909) or from Arabidopsis (e.g., the rd29A promoter (Kasuga et al. (1999) Nature Biotechnology 17:287-291).
environmental stress-inducible promoters include promoters from the following genes: Rab21, Wsi18, Lea3, Uge1, Dip1, and R1G1B in rice (Yi et al. (2010) Planta 232:743-754)

Plant Tissue-Specific Promoters
Epidermal-specific promoters
   Arabidopsis LTP1 promoter (Thoma et al. (1994) Plant Physiol. 105(1):35-45),
   CER1 promoter (Aarts et al. (1995) Plant Cell 7:2115-27),
   CER6 promoter (Hooker et al. (2002) Plant Physiol 129:1568-80),
   tomato LeCER6 (Vogg et al. (2004) J. Exp Bot. 55:1401-10).
Guard cell-specific promoters
   (Li et al (2005) Science China C Life Sci. 48:181-186).
seed promoters.
   MAC1 from maize (Sheridan (1996) Genetics 142:1009-1020);
   Cat3 from maize (GenBank No. L05934, Abler (1993) Plant Mol. Biol. 22:10131-1038);
   vivparous-1 from Arabidopsis (Genbank No. U93215);
   atmyc1 from Arabidopsis (Urao (1996) Plant Mol. Biol. 32:571-57; Conceicao (1994) Plant 5:493-505);
   napA from Brassica napus (GenBank No. J02798, Josefsson (1987) JBL 26:12196-1301);
   napin gene family from Brassica napus (Sjodahl (1995) Planta 197:264-271).
vegetative tissues, such as leaves, stems, roots and tubers,
   patatin, Kim (1994) Plant Mol. Biol. 26:603-615: Martin (1997) Plant J. 11:53-62.
   ORF 13 promoter from Agrobacterium rhizogenes that exhibits high activity in roots
   tarin promoter of the gene encoding a globulin from a major taro (Colocasia esculenta L. Schott) corm protein family,
   tarin (Bezerra (1995) Plant Mol. Biol. 28:137-144):
   curculin promoter active during taro corm development (de Castro (1992) Plant Cell 4:1549-1559)
   tobacco root-specific gene TobRB7, whose expression is localized to root meristem and immature central cylinder regions (Yamamoto (1991) Plant Cell 3:371-382).
Leaf-specific promoters,
   ribulose biphosphate carboxylase (RBCS) promoters-tomato RBCS1, RBCS2 and RBCS3A
   light harvesting chlorophyll a/b binding protein gene promoter, see, e.g., Shiina (1997) Plant Physiol. 115:477-483; Casal (1998) Plant Physiol. 116:1533-1538.
   Arabidopsis thaliana myb-related gene promoter (Atmyb5) Li (1996) FEBSLett. 379:117-121, is leaf-specific.
   leaf promoter identified in maize by Busk (1997) Plant J. 11:1285-1295,
meristematic (root tip and shoot apex) promoters.
   “SHOOTMERISTEMLESS” and “SCARECROW” promoters, Di Laurenzio (1996) Cell 86:423-433; and, Long (1996) Nature 379:66-69;
   3-hydroxy-3-methylglutaryl coenzyme A reductase HMG2 gene, (see, e.g., Enjuto (1995) Plant Cell. 7:517-527).
   kn1-related genes from maize and other species Granger (1996) Plant Mol. Biol. 31:373-378; Kerstetter (1994) Plant Cell 6:1877-1887; Hake (1995) Philos. Trans. R. S c. Lond. B. Biol. Sci. 350:45-51.
   Arabidopsis thaliana KNAT1 promoter (see, e.g., Lincoln (1994) Plant

Cell 6:1859-1876)

### Bacterial Promoters

T7
T3
lac operon promoters
trp
tac (hybrid of trp and lac promoters)
gpt
lambda PR,
lambda PL
sigma. 70 promoters (e.g., inducible pBad/araC promoter, Lux cassette right promoter, modified lambda Prm promote, plac Or2-62 (positive), pBad/AraC with extra REN sites, pBad, P(Las) TetO, P(Las) CIO, P(Rhl), Pu, FecA, pRE, cadC, hns, pLas, pLux), a “s” promoter (e.g., Pdps),
sigma. 32 promoters (e.g., heat shock)
sigma. 54 promoters (e.g., glnAp2);
negatively regulated E. coli promoters such as negatively regulated .sigma. 70 promoters (e.g., Promoter (PRM+), modified lambda Prm promoter, TetR-TetR-4C P(Las) TetO, P(Las) CIO, P(Lac) IQ, RecA_DlexO_DLac01, dapAp, FecA, Pspac-hy, pel, plux-cl, plux-lac, CinR, CinL, glucose controlled, modified Pr, modified Prm+, FecA, Pcya, rec A (SOS), RecA (SOS), EmrR_regulated, Bet1_regulated, pLac_lux, pTet_Lac, pLac/Mnt, pTet/Mnt, LsrA/cl, pLux/cl, LacI, LacIQ, pLacIQ1, pLas/cl, pLas/Lux, pLux/Las, pRecA with LexA binding site, reverse BBa_R0011, pLacl/ara-1, pLaclq, rrnB PI, cadC, hns, PfhuA, pBad/araC, nhaA, OmpF, RcnR),
sigma. S promoters (e.g., Lutz-Bujard LacO with alternative sigma factor .sigma. 38),
sigma. 32 promoters (e.g., Lutz-Bujard LacO with alternative sigma factor sigma. 32),
sigma. 54 promoters (e.g., glnAp2);
negatively regulated B. subtilis promoters such as repressible B. subtilis.sigma. A promoters (e.g., Gram-positive IPTG-inducible, Xyl, hyper-spank),
sigma. promoters, and the BioFAB promoters disclosed in Mutalik V K et al (Nature Methods, 2013, 10: 354-360, see in particular the supplementary data) as well as on the BioFAB website (http://biofab.synberc.org/data).

### Cell Types

Promoters used in a subject nucleic acid can be operable in a desired cell type and/or category of cells. For example, in some cases the promoters are operable in a prokaryotic cell and in other cases the promoters are operably in eukaryotic cells. For example, in some cases the promoters are operable in eukaryotic cells. In some cases, the promoters are plant promoters and in some cases they are animal (e.g., insect or mammalian) promoters. For all of the promoters listed herein (including in the Tables)—the corresponding cells/cell types can be used as host cells (target cells) and visa versa. Meaning for all of the types of cells listed herein a subject vector can include one or more promoters (e.g., a first and second promoter) that are operable in that cell type. For examples of promoters from a variety of different organisms, see Tables 4-6.

Host cells (also referred to as “target cells”) can be ex vivo (e.g., fresh isolate-early passage), in vivo, or in culture in vitro (e.g., immortalized cell line). In some cases, the targeted nucleic acid is chromosomal (e.g., the host cell's genome) and in some cases the targeted nucleic acid is from a pathogen, e.g., the genome of a pathogen within the host cell. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

Suitable host cells (which can comprise target nucleic acids such as genomic DNA) include, but are not limited to: a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell of a vertebrate animal (e.g., a fish, an amphibian, a reptile, a bird, a mammal); a cell of a mammal (e.g., a cell of a rodent; a cell of a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuña, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Suitable host cells (which can comprise target nucleic acids such as genomic DNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell of a vertebrate animal (e.g., a fish, an amphibian, a reptile, a bird, a mammal); a cell of a mammal (e.g., a cell of a rodent; a cell of a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuña, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells of any organism are of interest (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell of an invertebrate animal (e.g. fruit fly, cnidarian, echinoderm, nematode, etc.), a cell of a vertebrate animal (e.g., fish, amphibian, reptile, bird, mammal), a cell of a mammal, a cell of a rodent, a cell of a human, a cell of a non-human primate, etc.). As noted above—in some cases a target cell is in vivo and therefore a subject nucleic acid or protein (e.g., a subject vector) can be administered to an individual (e.g., a mammal, a rat, a mouse, a pig, a primate, a non-human primate, a human, etc.). In some case, such an administration can be for the purpose of treating and/or preventing a disease, e.g., by editing the genome of targeted cells

Cells of any eukaryotic organism are of interest (e.g. a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell of an invertebrate animal (e.g. fruit fly, cnidarian, echinoderm, nematode, etc.), a cell of a vertebrate animal (e.g., fish, amphibian, reptile, bird, mammal), a cell of a mammal, a cell of a rodent, a cell of a human, a cell of a non-human primate, etc.). As noted above—in some cases a target cell is in vivo and therefore a subject nucleic acid or protein (e.g., a subject vector) can be administered to an individual (e.g., a mammal, a rat, a mouse, a pig, a primate, a non-human primate, a human, etc.). In some case, such an administration can be for the purpose of treating and/or preventing a disease, e.g., by editing the genome of targeted cells.

Non-limiting examples of cells (target cells) include: a eukaryotic cell, a cell of a single-cell eukaryotic organism, a protozoa cell, a cell of a plant (e.g., cells from plant crops, fruits, vegetables, grains, soy bean, corn, maize, wheat, seeds, tomatos, rice, cassava, sugarcane, pumpkin, hay, potatos, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell of a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell of a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

Non-limiting examples of cells (target cells) include: a prokaryotic cell, eukaryotic cell, a bacterial cell, an archaeal cell, a cell of a single-cell eukaryotic organism, a protozoa cell, a cell of a plant (e.g., cells from plant crops, fruits, vegetables, grains, soy bean, corn, maize, wheat, seeds, tomatos, rice, cassava, sugarcane, pumpkin, hay, potatos, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell of a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell of a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A plant cell can be a cell of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

A plant cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

### Guide RNA

In some embodiments, a subject composition or method includes a guide RNA. For example, in some cases a subject composition or method (e.g., a vector or vector system) includes an expression cassette that includes a promoter operably linked to a sequence encoding a guide RNA. In some such cases the promoter is an RNA polymerase III promoter (e.g. U6, H1), which can be used to express non-coding RNAs in eukaryotic cells.

A “guide RNA” is nucleic acid that binds to a Cas protein (e.g., a Class 2 CRISPR-Cas effector protein such as Cas9 or Cas12), thus forming a CRISPR complex (a protein-RNA effector complex)—and can target the CRISPR complex to a specific ‘on-target’ target sequence within a target nucleic acid (e.g., genomic DNA, e.g., eukaryotic or prokaryotic genomic DNA). It is to be understood that in some cases, a hybrid DNA/RNA can be made such that a guide RNA includes DNA bases in addition to RNA bases—but the term “guide RNA” is still used herein to encompass such hybrid molecules.

A guide RNA provides target specificity to the CRISPR complex by including a targeting segment, which includes a guide sequence (also referred to herein as a targeting sequence), which is a nucleotide sequence that is complementary to a sequence of a target nucleic acid. Thus, a subject guide RNA includes (i) a guide sequence (also referred to as a “spacer” or “targeting sequence”) that hybridizes to a target sequence (also referred to as a “protospacer”) of a target nucleic acid, e.g., target DNA; and (ii) a constant region (e.g., a region that is adjacent to the guide sequence and binds to the Cas protein). A “constant region” can also be referred to herein as a “protein-binding segment” or a “handle.” Thus, the location of an on-target event (e.g., target DNA cleavage, transcription modulation, DNA methylation, histone modification) is in effect determined by the guide sequence of the guide RNA. CRISPR complex mediated events that take place at a location that is not a 100% match with the guide sequence is referred to herein as an off-target event.

A guide RNA can be referred to by the protein to which it corresponds. For example, when the guide RNA binds to and guides a class 2 CRISPR/Cas effector protein, the guide RNA can be referred to as a “class 2 guide RNA.” Likewise, when the class 2 CRISPR/Cas effector protein is a Cas9 protein, the corresponding guide RNA can be referred to as a “Cas9 guide RNA.” As another example, when the class 2 CRISPR/Cas effector protein is a Cpf1 (Cas12a) protein, the corresponding guide RNA can be referred to as a “Cpf1 guide RNA” or “Cas12a guide RNA.”

In some embodiments, a guide RNA includes two separate nucleic acid molecules: an “activator” (e.g., a tracrRNA) and a “targeter” (e.g., a crRNA) and is referred to herein as a “dual guide RNA”, a “double-molecule guide RNA”, a “two-molecule guide RNA”, or a “dgRNA.” In some embodiments, the guide RNA is one molecule. For example, for some class 2 CRISPR/Cas systems the corresponding guide RNA is naturally a single molecule, while for other class 2 CRISPR/Cas systems the corresponding guide RNA is naturally two separate molecules (e.g., a crRNA and a tracrRNA)—and the two molecules (an activator, e.g., tracrRNA, and a targeter, e.g., a crRNA) can be covalently linked to one another, e.g., via chemical linkage or intervening nucleotides. When the guide RNA is one molecule, the guide RNA can be referred to as a “single guide RNA”, a “single-molecule guide RNA,” a “one-molecule guide RNA”, or simply “sgRNA.” “Guide RNA” (or “gRNA”) is a generic term that encompasses dual guide and single guide formats.

The guide sequence has complementarity with (hybridizes to) a target sequence of the target nucleic acid (e.g., target DNA). In some cases, the guide sequence is 15-28 nucleotides (nt) in length (e.g., 15-26, 15-24, 15-22, 15-20, 15-18, 16-28, 16-26, 16-24, 16-22, 16-20, 16-18, 17-26, 17-24, 17-22, 17-21, 17-20, 17-19, 17-18, 18-26, 18-24, 18-22, 18-20, or 19-21 nt in length). In some cases, the guide sequence is 18-24 nucleotides (nt) in length. In some cases, the guide sequence is 17-18 nucleotides (nt) in length. In some cases, the guide sequence is at least 15 nt long (e.g., at least 16, 18, 20, or 22 nt long). In some cases, the guide sequence is at least 17 nt long. In some cases, the guide sequence is at least 18 nt long. In some cases, the guide sequence is at least 20 nt long. In some cases, the guide sequence is 20 nt long.

In some cases, the constant region (also referred to as a scaffold) of a guide

RNA is 15 or more nucleotides (nt) in length (e.g., 18 or more, 20 or more, 21 or more, 22 or more, 23 or more, 24 or more, 25 or more, 26 or more, 27 or more, 28 or more, 29 or more, 30 or more, 31 or more nt, 32 or more, 33 or more, 34 or more, 35 or more, 40 or more, 45 or more, 50 or more, 60 or more, 70 or more, 80 or more, 90 or more, or 100 or more nt in length). In some cases, the constant region of a guide RNA is 18 or more nt in length.

Guide RNAs with various modifications to increase efficiency relative to naturally existing guide RNAs (e.g., via chemical modifications, alterations in the spacer length, sequence modifications in the spacer or scaffold, fusion with additional DNA or RNA components, partial replacement with DNA, and the like) are known in the art and are readily available to one of ordinary skill in art. See, e.g., Moon et al., Trends Biotechnol. 2019 August; 37(8):870-881, “Improving CRISPR Genome Editing by Engineering Guide RNAs”. The term “guide RNA” as used herein encompasses such modifications and any convenient guide RNA can be used with the methods and compositions disclosed herein (e.g., as part of a subject system—for example as RNA or as encoded by a subject nucleic acid).

### “Protospacer Adjacent Motif” (PAM)

A wild type CRISPR/Cas effector protein (e.g., Cas9 protein) normally has nuclease activity that cleaves a target nucleic acid (e.g., a double stranded DNA (dsDNA)) at a target site defined by (i) the region of complementarity between the guide sequence of the guide RNA and the target nucleic acid; and (ii) a short motif referred to as the “protospacer adjacent motif” (PAM) in the target nucleic acid. For example, when a Cas9 protein binds to a dsDNA target nucleic acid, the PAM sequence that is recognized (bound) by the Cas9 polypeptide is present on the non-complementary strand (the strand that does not hybridize with the targeting segment of the guide nucleic acid) of the target DNA. CRISRPR/Cas (e.g., Cas9) proteins from different species can have different PAM sequence preferences.

For additional information related to programmable gene editing tools (e.g., CRISPR/Cas RNA-guided proteins such as Cas9, CasX, CasY, and Cpf1, CRISPR/Cas guide RNAs, and PAMs) refer to, for example, Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 July 34(7):768-73; Shmakov et al., Nat Rev Microbiol. 2017 March; 15(3):169-182; Kleinstiver, B. P. et al, Nature 529, 490-495 (2016); Slaymaker, I. M. et al., Science 351, 84-88 (2016); as well as U.S. patent application publication Nos. 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 8,906,616; 8,895,308; 8,889,418; 8,889,356; 8,871,445; 8,865,406; 8,795,965; 8,771,945; 8,697,359; 10,000,772; 10,113,167; 10,227,611; 10,266,850; 10,301,651; 10,308,961; 10,337,029; 10,351,878; 10,358,658; 10,358,659; 10,385,360; 10,400,253; 10,407,697; 10,415,061; 10,421,980; 10,428,352; 10,443,076; 10,487,341; 10,513,712; 10,519,467; 10,526,619, 11,124,783; 11,098,297; 11,091,798; 11,060,078; and 11,060,115; all of which are hereby incorporated by reference in their entirety.

### Vectors

A “vector” or “expression vector” is a replicon, such as plasmid, phage, virus, or cosmid, to which another DNA segment, i.e. an “insert”, may be attached so as to bring about the replication and/or expression of the attached segment in a cell. An “expression cassette” comprises a DNA sequence (coding or non-coding) operably linked to a promoter. In some cases, a subject vector is a viral vector (e.g., AAV, lentivirus, adenovirus). In some cases, a subject vector includes an origin of replication (e.g., can be a plasmid).

In some cases, both an Acr protein and its target Cas protein (the protein that the Acr inhibits) is present in a single vector—which ensures that all cells receiving the Cas protein (e.g., an endonuclease such as Cas9, Cas12a, and the like) will also express the Acr “off-switch”. Whether or not both proteins (Acr and Cas) are present on the same nucleic acid, the translation of one or both proteins can be regulated by a translational control element in order to achieve a proper balance (expression level ratio) between the two proteins.

Vectors may be provided directly to a target host cell (target cell). In other words, the cells are contacted with vectors comprising the subject nucleic acids (e.g., recombinant expression vectors) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors (e.g., adeno-associated virus (AAV)).

In some embodiments, a subject vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments a subject vector is an AAV vector. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

In some embodiments a subject vector is an integrative vector, e.g., integrates into the genome of a target cell.

By a “recombinant AAV vector”, or “rAAV vector” it is meant an AAV virus or AAV viral chromosomal material comprising a polynucleotide sequence not of AAV origin (i.e., a polynucleotide heterologous to AAV), typically a nucleic acid sequence of interest to be integrated into the cell following the subject methods. In general, the heterologous polynucleotide is flanked by at least one, and generally by two AAV inverted terminal repeat sequences (ITRs). In some instances, the recombinant viral vector also comprises viral genes important for the packaging of the recombinant viral vector material. By “packaging” it is meant a series of intracellular events that result in the assembly and encapsidation of a viral particle, e.g. an AAV viral particle. Examples of nucleic acid sequences important for AAV packaging (i.e., “packaging genes”) include the AAV “rep” and “cap” genes, which encode for replication and encapsidation proteins of adeno-associated virus, respectively. The term rAAV vector encompasses both rAAV vector particles and rAAV vector plasmids.

A “viral particle” refers to a single unit of virus comprising a capsid encapsidating a virus-based polynucleotide, e.g. the viral genome (as in a wild type virus), or, e.g., the subject targeting vector (as in a recombinant virus). An “AAV viral particle” refers to a viral particle composed of at least one AAV capsid protein (typically by all of the capsid proteins of a wild-type AAV) and an encapsidated polynucleotide AAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

A rAAV virion can be constructed using methods that are well known in the art. See, e.g., Koerber et al. (2009) Mol. Ther. 17:2088; Koerber et al. (2008) Mol Ther. 16:1703-1709; U.S. Pat. Nos. 7,439,065, 6,951,758, and 6,491,907. For example, the heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce rAAV virions, an AAV expression vector can be introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechnigues 6:742-751), liposome mediated gene transfer (Mannino et al. (1988) BioTechniques 6:682-690), lipid-mediated transduction (Feigner et al. (1987) Proc. Natl. Acad. Sci. USA 84:7413-7417), and nucleic acid delivery using high-velocity microprojectiles (Klein et al. (1987) Nature 327:70-73).

Suitable cells for producing rAAV virions include microorganisms, yeast cells, insect cells, and mammalian cells, that can be, or have been, used as recipients of a heterologous DNA molecule. Cells from the stable human cell line, 293 (readily available through, e.g., the American Type Culture Collection under Accession Number ATCC CRL1573) can be used. For example, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a convenient platform in which to produce rAAV virions. Methods of producing an AAV virion in insect cells are known in the art, and can be used to produce a subject rAAV virion. See, e.g., U.S. Patent Publication No. 2009/0203071; U.S. Pat. No. 7,271,002; and Chen (2008) Mol. Ther. 16:924.

AAV virus that is produced may be replication competent or replication-incompetent. A “replication-competent” virus (e.g. a replication-competent AAV) refers to a phenotypically wild-type virus that is infectious, and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

A detailed discussion of delivery methods and formulations is presented elsewhere herein.

As noted elsewhere herein, proteins may instead be provided to cells as RNA (e.g., an RNA comprising the translational control element as discussed elsewhere herein). Methods of introducing RNA into cells are known in the art and may include, for example, direct injection, transfection, or any other method used for the introduction of DNA.

In some cases, one or more proteins (e.g., a Cas effector protein) can be introduced into a cell as a polypeptide (as opposed to a nucleic acid). For example, one protein coding sequence (such as an Acr protein coding sequence) can be introduced as nucleic acid (RNA or DNA) where the protein coding sequence is operably linked to a translational control element; and the other protein (e.g., a Cas effector) is introduced as a polypeptide. Such a polypeptide may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. Examples of linkers are discussed elsewhere herein in a different context, but such linkers can be used in any convenient context including this one.

In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. I F2 domain, GST domain, GRPE domain, and the like. The polypeptide may be formulated for improved stability. For example, the peptides may be PEGylated, where the polyethyleneoxy group provides for enhanced lifetime in the blood stream.

### Vector Systems (e.g., Split Cas9)

Provided are coordinated delivery systems that include more than one vector. In some cases, a Cas protein can be split into two separate partial-proteins that can function as whole protein when the two parts are brought together. For example, the two parts can each be fused to a dimerization domain and dimerization can be induced in order to form a functional Cas protein. As an illustrative example, Cas9 can be split into two separate half-proteins, and its dimerization into an active form can be made to be dependent upon a small molecule dimerizer (e.g. rapamycin)—see, e.g., Zetsche et al., “A split-Cas9 architecture for inducible genome editing and transcription modulation” Nature Biotechnol. 33:139-140 (2015).

Thus, two separate portions of a Cas protein (e.g., Cas9) can in some cases be present on two separate vectors—or can be present on the same vector but be operably linked to different promoters.

Thus, in some cases the coordinated delivery system includes two Cas encoding sequences, a first portion (e.g., of a Class 2 Cas effector protein such as a Cas9); and a second portion. The first portion and second portion of the Cas protein (e.g., a Class 2 Cas effector protein) together form a functional Cas protein. In such cases the Acr protein is an inhibitor of the functional Cas protein

### Methods

The present disclosure provides a method for nucleic acid targeting (e.g., for cleaving DNA such as in genome editing applications), where the method includes contacting a target nucleic acid with a subject system. In some cases, the contact is in a cell-free environment in vitro. In some cases, the contacting occurs in a cell, which can be ex vivo, in vivo, or in vitro (e.g., a cell in culture). Thus, in some embodiments a subject method includes introducing a coordinated delivery system (where the coordinated delivery system includes a translational control element, e.g., one or more subject nucleic acids) into a host cell, whereby the Acr protein and the Cas protein are expressed (at the protein level) in the host cell at a ratio relative to one another such that the ratio of on-target to off-target nucleic acid activity (e.g., cleavage) that results from said introducing is increased relative to the ratio of on-target to off-target nucleic acid targeting that would result in the absence of the Acr protein. In some cases, the Acr protein and the Cas protein are expressed (at the protein level) in the host cell at a ratio relative to one another such that the ratio of on-target to off-target nucleic acid activity (e.g., cleavage) that results from said introducing is increased relative to the ratio of on-target to off-target nucleic acid targeting that would result in the absence of the translational control element. In some cases, the ratio of on-target to off-target nucleic acid targeting that results is caused by an increase in on-target activity. In some cases, the ratio of on-target to off-target nucleic acid targeting that results is caused by a decrease in off-target activity. In some cases, the ratio of on-target to off-target nucleic acid targeting that results is caused by both an increase in on-target activity and a decrease in off-target activity.

The targeted cell (the host cell) can be any desired cell/cell type. Examples of suitable cells and promoters are described in detail elsewhere herein (see, e.g., the “promoter” section. For example, in some cases the cell is a prokaryotic cell, a plant cell, an insect cell, a vertebrate cell, an invertebrate cell, an animal cell, a mammalian cell, or a human cell. For example, in some cases the cell is a eukaryotic cell, a plant cell, an insect cell, a vertebrate cell, an invertebrate cell, an animal cell, a mammalian cell, or a human cell. In some cases, the cell is ex vivo. In some cases, the cell is in vivo. In some cases, the cell is in culture in vitro.

In some embodiments the nucleic acid targeted by the CRISPR complex (on-target events) is the host cell's genome. In some embodiments the nucleic acid targeted by the CRISPR complex (on-target events) is the genome of a pathogen (e.g., a virus)—in some cases the pathogen is in the host cell. In some embodiments the nucleic acid targeted by the CRISPR complex (on-target events) is the genome of a pathogen (virus, bacteria, and the like)—in some cases the pathogen is in the host cell. In some embodiments the nucleic acid targeted by the CRISPR complex (on-target events) is and RNA molecule. In some cases, the on-target nucleic acid targeting alters expression of a protein within the host cell (e.g., via decreasing transcription of the mRNA). In some cases, the on-target nucleic acid targeting alters expression of an RNA (e.g., a noncoding RNA, an mRNA, a microRNA, and the like) within the host cell.

In some cases, the on-target nucleic acid targeting activity of the CRISPR complex causes gene editing (e.g., correction of a genetic mutation in the host cell genome). In some cases, the on-target nucleic acid targeting activity of the CRISPR complex causes alteration of a genetic site (editing) from a disease-associated sequence to a healthy-associated sequence—e.g., correction of the Huntington's Disease (HD), Duchenne Muscular Dystrophy (DMD), or Alpha-1 antitrypsin Disease (AATD) disease-causing alleles into alleles not associated with (non-causative of) disease.

As noted elsewhere herein, the location of an on-target event (e.g., target DNA cleavage/editing) is in effect determined by the guide sequence of the guide RNA. CRISPR complex mediated events that take place at a location that is not a 100% match with the guide sequence are referred to herein as off-target events. Any convenient method can be used to measure on-target and off-target events and the selection of method will depend on the type of CRISPR complex used and desired outcome of the complex's activity (e.g., when using a nickase protein, when performing double stranded target cleavage, when using a donor polynucleotide—which can edit the target by introducing known heterologous sequence; when not using a donor polynucleotide—which can lead to numerous different indels, etc.)), Examples of suitable assays include but are not limited to: mismatch cleavage assays (e.g., surveyor assay, T7E1 mismatch assay), PCR assays; PCR/sequencing assays, direct sequencing assays such as next generation sequencing, and the like (and any combination thereof). Sequencing assays or alternative expression assays such as qRT-PCR and/or microarray analysis can be used when the activity of the CRISPR complex results in an alteration of expression of a target sequence (e.g., when a promoter sequence is targeted, when a coding sequence is targeted and the new sequence is susceptible to nonsense-mediated decay, and the like). Various assays exist to test for on-target and off-target activities and any desired assay or combination of assays can be used.

In some cases, a desirable outcome (an acceptable outcome achieved by a selected promoter combination) is an outcome in which the off-target rate is less than 100 off-target events detected per cell population (e.g., off-target cleavage events such as insertion/deletions (indels) detected per cell population). In some such cases, the number of cells in the cell population is in a range of from 104 to 106 (e.g., in some cases the number of cells in the cell population is about 10⁵ cells). In some cases, a desirable outcome (an acceptable outcome achieved by a selected promoter combination) is an outcome in which the off-target rate is less than 90 off-target events detected per cell population (e.g., less than 80, less than 70, less than 60, less than 50, less than 40, less than 30, less than 20, less than 10, or less than 5 off-target events per cell). In some cases, a desirable outcome (an acceptable outcome achieved by a selected promoter combination) is an outcome in which the off-target rate is less than 50 off-target events detected per cell population (e.g., less than 40, less than 30, less than 20, less than 10, or less than 5 off-target events per cell).

In some cases, a desirable outcome (an acceptable outcome achieved by a selected promoter combination) is an outcome in which the off-target rate is less than 100 off-target events detected per 10⁵ cells. In some cases, a desirable outcome (an acceptable outcome achieved by a selected promoter combination) is an outcome in which the off-target rate is less than 90 off-target events detected per 10⁵ cells (e.g., less than 80, less than 70, less than 60, less than 50, less than 40, less than 30, less than 20, less than 10, or less than 5 off-target events per cell). In some cases, a desirable outcome (an acceptable outcome achieved by a selected promoter combination) is an outcome in which the off-target rate is less than 50 off-target events detected per 10⁵ cells (e.g., less than 40, less than 30, less than 20, less than 10, or less than 5 off-target events per cell).

In some cases, a desirable outcome (an acceptable outcome achieved by a selected promoter combination) is an outcome in which less than 50% (e.g., less than 45%, less than 40%, or less than 35%) of the total measured nucleic acid targeting events (e.g., cleavage) are off-target events. In other words, in some cases the ratio of on-target to off-target events (e.g., measured on-target to off-target events) is greater than 1 (e.g., greater than 1.2, greater than 1.5, greater than 1.8, greater than 2, greater than 2.2, or greater than 2.5). In some cases, the events can be measured after passaging the host cell (e.g., in some cases for 10 or more generations) after the Acr and Cas proteins are introduced. Thus, in some cases a desirable outcome is an outcome in which, after passaging the host cell (e.g., for 10 or more generations) after the Acr and Cas proteins are introduced, less than 50% (e.g., less than 45%, less than 40%, or less than 35%) of the total measured nucleic acid targeting events (e.g., cleavage) are off-target events. In other words, in some such cases the ratio of on-target to off-target events (e.g., measured on-target to off-target events) is greater than 1 (e.g., greater than 1.2, greater than 1.5, greater than 1.8, greater than 2, greater than 2.2, or greater than 2.5).

As noted above, off-target sites can in some cases be predicted. Generally, the rate (frequency) of off-target activity (e.g., cleavage/editing) will vary from site to site, e.g., when measuring rates of activity using a population of cells. As such, in some cases, a desirable outcome (an acceptable outcome achieved by a selected promoter combination) is an outcome in which the measured frequency of off-target events is less than 50% (e.g., less than 45%, less than 40%, less than 35%, less than 30%, less than 25%, less than 20%, less than 15%, less than 10%, less than 5%, less than 2%, or less than 1%) when compared to the off-target events measured (or expected) in the absence of the Acr protein. As an illustrative example, on can measure the frequency of off-target events at one particular predicted or known off-target site (or at any number of off-target sites—predicted/known or not predict/known) in the presence of the Acr protein (meaning—when the experiment is performed in the present of the Acr protein) and in the absence of the Acr protein—and the number of off-target events when the Acr protein is present is less than 50% (e.g., less than 45%, less than 40%, or less than 35%, less than 30%, less than 25%, less than 20%, less than 15%, less than 10%, less than 5%, less than 2%, or less than 1%) compared to the number of off-target evens when the Acr protein is absent. As an additional illustrative example of the above, if 100 total off-target events are measured when the method is performed in the presence of the Acr protein, but 200 such events are measured (or expected) in the absence of the Acr protein, then the outcome would be a measured frequency of off-target events in the presence of the Acr protein that is 50% when compared to the off-target events in the absence of the Acr protein.

In some cases, a desirable outcome (an acceptable outcome achieved by a subject translational control element, e.g., an IRES, 2A peptide, non-AUG start codon) is an outcome in which the off-target rate is less than 100 off-target events detected per cell population (e.g., off-target cleavage events such as insertion/deletions (indels) detected per cell population). In some such cases, the number of cells in the cell population is in a range of from 104 to 106 (e.g., in some cases the number of cells in the cell population is about 10⁵ cells). In some cases, a desirable outcome (an acceptable outcome achieved by a subject translational control element, e.g., an IRES, 2A peptide, non-AUG start codon) is an outcome in which the off-target rate is less than 90 off-target events detected per cell population (e.g., less than 80, less than 70, less than 60, less than 50, less than 40, less than 30, less than 20, less than 10, or less than 5 off-target events per cell). In some cases, a desirable outcome (an acceptable outcome achieved by a subject translational control element, e.g., an IRES, 2A peptide, non-AUG start codon) is an outcome in which the off-target rate is less than 50 off-target events detected per cell population (e.g., less than 40, less than 30, less than 20, less than 10, or less than 5 off-target events per cell).

In some cases, a desirable outcome (an acceptable outcome achieved by a subject translational control element, e.g., an IRES, 2A peptide, non-AUG start codon) is an outcome in which the off-target rate is less than 100 off-target events detected per 10⁵ cells. In some cases, a desirable outcome (an acceptable outcome achieved by a subject translational control element, e.g., an IRES, 2A peptide, non-AUG start codon) is an outcome in which the off-target rate is less than 90 off-target events detected per 10⁵ cells (e.g., less than 80, less than 70, less than 60, less than 50, less than 40, less than 30, less than 20, less than 10, or less than 5 off-target events per cell). In some cases, a desirable outcome (an acceptable outcome achieved by a subject translational control element, e.g., an IRES, 2A peptide, non-AUG start codon) is an outcome in which the off-target rate is less than 50 off-target events detected per 10⁵ cells (e.g., less than 40, less than 30, less than 20, less than 10, or less than 5 off-target events per cell).

In some cases, a desirable outcome (an acceptable outcome achieved by a subject translational control element, e.g., an IRES, 2A peptide, non-AUG start codon) is an outcome in which less than 50% (e.g., less than 45%, less than 40%, or less than 35%) of the total measured nucleic acid targeting events (e.g., cleavage) are off-target events. In other words, in some cases the ratio of on-target to off-target events (e.g., measured on-target to off-target events) is greater than 1 (e.g., greater than 1.2, greater than 1.5, greater than 1.8, greater than 2, greater than 2.2, or greater than 2.5). In some cases, the events can be measured after passaging the host cell (e.g., in some cases for 10 or more generations) after the Acr and Cas proteins are introduced. Thus, in some cases a desirable outcome is an outcome in which, after passaging the host cell (e.g., for 10 or more generations) after the Acr and Cas proteins are introduced, less than 50% (e.g., less than 45%, less than 40%, or less than 35%) of the total measured nucleic acid targeting events (e.g., cleavage) are off-target events. In other words, in some such cases the ratio of on-target to off-target events (e.g., measured on-target to off-target events) is greater than 1 (e.g., greater than 1.2, greater than 1.5, greater than 1.8, greater than 2, greater than 2.2, or greater than 2.5).

As noted above, off-target sites can in some cases be predicted. Generally, the rate (frequency) of off-target activity (e.g., cleavage/editing) will vary from site to site, e.g., when measuring rates of activity using a population of cells. As such, in some cases, a desirable outcome (an acceptable outcome achieved by a subject translational control element, e.g., an IRES, 2A peptide, non-AUG start codon) is an outcome in which the measured frequency of off-target events is less than 50% (e.g., less than 45%, less than 40%, less than 35%, less than 30%, less than 25%, less than 20%, less than 15%, less than 10%, less than 5%, less than 2%, or less than 1%) when compared to the off-target events measured (or expected) in the absence of the Acr protein. As an illustrative example, on can measure the frequency of off-target events at one particular predicted or known off-target site (or at any number of off-target sites—predicted/known or not predict/known) in the presence of the Acr protein (meaning—when the experiment is performed in the present of the Acr protein) and in the absence of the Acr protein—and the number of off-target events when the Acr protein is present is less than 50% (e.g., less than 45%, less than 40%, or less than 35%, less than 30%, less than 25%, less than 20%, less than 15%, less than 10%, less than 5%, less than 2%, or less than 1%) compared to the number of off-target evens when the Acr protein is absent. As an additional illustrative example of the above, if 100 total off-target events are measured when the method is performed in the presence of the Acr protein, but 200 such events are measured (or expected) in the absence of the Acr protein, then the outcome would be a measured frequency of off-target events in the presence of the Acr protein that is 50% when compared to the off-target events in the absence of the Acr protein.x

In some cases, a desirable outcome achieved by use of one or more translational control elements described elsewhere herein (e.g., an IRES, 2A peptide, non-AUG start codon) is an outcome in which the ratio of on-target to off-target events is improved as compared to an alternative CRISPR/Cas editing system. In some cases, the comparison is made to a system with the same Cas nuclease lacking an Acr protein or lacking an Acr protein that interacts with the selected Cas protein. In some cases, the comparison is made to a system with the same Cas nuclease and the same Acr protein but lacking the translational control element(s) regulating the Cas protein or the Acr protein. In some cases, the improvement in the ratio of on-target to off-target events is greater than 1 (e.g., greater than 1.2, greater than 1.5, greater than 1.8, greater than 2, greater than 2.2, or greater than 2.5). In some cases, the improvement in the ratio is at least 2×, 2.5×, 3×, 4×, 5×, or more than 5×.

In some cases, the off-target sites are predicted and/or known sties, and in some cases the off-target sites can be identified after the fact (e.g., based on a genome-wide hunt such as can be achieved using high throughput/next generation sequencing methods such as RNA or DNA sequencing methods).

In some cases, a number of pilot experiments are first performed to determine what the desirable translational control element and arrangement of components is for a particular CRISPR complex of interest in order to achieve a desired ratio of on-target to off-target events (see, e.g., FIG. 8, FIG. 12, and FIG. 18). For example, a plurality (e.g., a library) of translational control elements and arrangements can be tested for expressing the Acr and Cas proteins, and those combinations that achieve the most desirable activity outcomes (e.g., most desired balance of on-target to off-target activity) can then be selected for construction of a subject nucleic acid system (e.g., a single vector). Either way, once preferred combinations are determined, protein expression levels can be measured in the host cells to determine desirable ratios of Acr protein to Cas protein expression if so desired.

### Delivery

As noted above, in some embodiments both the Cas protein and the Acr protein will be delivered to a host cell as DNA and in some such cases the sequence encoding the two proteins will be present on the same nucleic acid (e.g., DNA vector) or on separate nucleic acids. However, in some embodiments a subject protein (e.g., Cas protein and/or Acr protein) is not provided as a DNA vector. For example, either protein (or both) can be introduced into a host cell as RNA encoding the protein. In such cases the RNA encoding the two proteins can be delivered in an appropriate ratio to achieve the desired affect (i.e., increased ratio of on-target to off-target CRISPR complex activity)—e.g., by decreasing off-target activity while retaining desirable on-target activity, and one or more translational control elements can be present on the RNAs.

As another example, either protein (or both) can be introduced into a host cell directly as proteins. In some such cases (e.g., if the Cas protein is a class 2 effector protein) the Cas protein can be delivered as an RNP (ribonucleoprotein complex) in which it is already complexed with an appropriate guide RNA. In such cases the other protein (e.g., the Acr protein) can be delivered as DNA or RNA and its coding sequence can be operably linked to a subject translational control element.

Thus, the Cas protein and the Acr protein can be delivered in any desired format (DNA, RNA, protein). For example, if the Cas protein is delivered as DNA, the Acr protein can be delivered as DNA, RNA, or protein; if the Cas protein is delivered as RNA, the Acr protein can be delivered as DNA, RNA, or protein; and if the Cas protein is delivered as protein, the Acr protein can be delivered as DNA or RNA. Likewise, if the Acr protein is delivered as DNA, the Cas protein can be delivered as DNA, RNA, or protein; if the Acr protein is delivered as RNA, the Cas protein can be delivered as DNA, RNA, or protein; and if the Acr protein is delivered as protein, the Cas protein can be delivered as DNA or RNA.

As would be readily understood by one of ordinary skill in the art, subject nucleic acids (e.g., vectors) and proteins can be delivered to cells using any convenient method. Methods of introducing nucleic acids and/or proteins into a host cell (e.g., prokaryotic cell, eukaryotic cell, plant cell, animal cell, insect cell, mammalian cell, human cell, and the like) are known in the art, and any convenient method can be used. Suitable methods include, e.g., viral infection (e.g., AAV, adenovirus, lentiviral), transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9), and the like.

In some cases, a protein of the present disclosure (e.g., Cas protein, Acr protein) is provided as a nucleic acid (e.g., an mRNA, a DNA, a plasmid, an expression vector, a viral vector, etc.) that encodes the protein. In some cases, a subject protein is provided directly as a protein (e.g., without an associated guide RNA or with an associate guide RNA, i.e., as a ribonucleoprotein complex). A subject protein can be introduced into a cell (provided to the cell) by any convenient method; such methods are known to those of ordinary skill in the art. As an illustrative example, a subject protein can be injected directly into a cell. As another example, a subject protein can be introduced into a cell (e.g, eukaryotic cell) via nucleofection; via a protein transduction domain (PTD) conjugated to the protein, etc.

In some cases, a subject protein is delivered to a cell (e.g., a target host cell) in a particle, or associated with a particle. In some cases, a subject protein is delivered with a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DM PC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DM PC 0, PEG 5, Cholesterol 5).

A subject protein (as RNA or DNA or protein) may be delivered using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used.

Lipidoid compounds (e.g., as described in US patent application 20110293703) are also useful in the administration of polynucleotides, and can be used to deliver a subject protein (or RNA or DNA encoding it). In one aspect, the aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver a subject protein or nucleic acid to a target cell. US Patent Publication No. 20130302401 relates to a class of poly(beta-amino alcohols) (PBAAs) that has been prepared using combinatorial polymerization.

Sugar-based particles may be used, for example GaINAc, as described with reference to WO2014118272 (incorporated herein by reference) and Nair, J K et al., 2014, Journal of the American Chemical Society 136 (49), 16958-16961) can be used to deliver a subject protein or nucleic acid to a target cell.

In some cases, lipid nanoparticles (LNPs) are used to deliver a subject protein or nucleic acid to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyI]-1,2-dimyristoyl-sn-glycol (PEG-S-DMG), and R-3-[(.omega.-methoxy-poly(ethylene glycol)2000) carbamoyl]-1,2-dimyristyloxlpropyl-3-amine (PEG-C-DOMG) may be used. A nucleic acid may be encapsulated in LNPs containing DLinDAP, DLinDMA, DLinK-DMA, and DLinKC2-DMA (cationic lipid:DSPC:CHOL:PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to deliver a subject protein or nucleic acid to a target cell.. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

In general, a “nanoparticle” refers to any particle having a diameter of less than 1000 nm. In some cases, nanoparticles suitable for use in delivering a subject protein or nucleic acid to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a a subject protein or nucleic acid to a target cell have a diameter of from 25 nm to 200 nm.

Nanoparticles suitable for use in delivering a subject protein or nucleic acid to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for use in delivering a subject protein or nucleic acid to a target cell. A prototype nanoparticle of semi-solid nature is the liposome.

In some cases, an exosome is used to deliver a subject protein or nucleic acid to a target cell. Exosomes are endogenous nano-vesicles that transport RNAs and proteins, and which can deliver RNA to the brain and other target organs.

In some cases, a liposome is used to deliver a subject protein or nucleic acid to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver a subject protein or nucleic acid to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver a subject protein or nucleic acid to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a subject protein or nucleic acid to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a subject protein or nucleic acid using a spontaneous vesicle formation procedure. The component molar ratio may be about 50/10/38.5/1.5 (DLin-KC2-DMA or C12-200/disteroylphosphatidyl choline/cholesterol/PEG-DMG).

A subject protein or nucleic acid may be delivered encapsulated in PLGA microspheres such as that further described in US published applications 20130252281 and 20130245107 and 20130244279.

Supercharged proteins can be used to deliver a subject protein or nucleic acid to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver a subject protein or nucleic acid to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

An implantable device can be used to deliver a subject protein or nucleic acid to a target cell (e.g., a target cell in vivo, where the target cell is a target cell in circulation, a target cell in a tissue, a target cell in an organ, etc.). An implantable device suitable for use in delivering a subject protein or nucleic acid to a target cell (e.g., a target cell in vivo, where the target cell is a target cell in circulation, a target cell in a tissue, a target cell in an organ, etc.) can include a container (e.g., a reservoir, a matrix, etc.) that comprises the subject protein or nucleic acid.

In some cases, desirable delivery systems provide for roughly uniform distribution and have controllable rates of release of their components (e.g., vectors, proteins, nucleic acids, drugs etc.). A variety of different media are described below that are useful in creating composition delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated include materials such as gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof. In some cases, subject compositions are delivered as therapeutic agents (e.g., administered to a subject in vivo).

In some cases, a carrier/medium can include a microparticle. Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids—in any combination thereof.

In some cases, a carrier/medium can include a liposome that is capable of attaching and releasing therapeutic agents (e.g., the subject nucleic acids and/or proteins). Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life.

In some embodiments, a cationic or anionic liposome is used as part of a subject composition or method, or liposomes having neutral lipids can also be used. Cationic liposomes can include negatively-charged materials by mixing the materials and fatty acid liposomal components and allowing them to charge-associate. The choice of a cationic or anionic liposome depends upon the desired pH of the final liposome mixture. Examples of cationic liposomes include but are not limited to: lipofectin, lipofectamine, and lipofectace.

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90.mu.m. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. II: 711-719 (1977).

Sustained or controlled release microsphere preparation can be prepared using an in-water drying method, where an organic solvent solution of a biodegradable polymer metal salt is first prepared. Subsequently, a dissolved or dispersed medium of a therapeutic agent can be added to the biodegradable polymer metal salt solution. The weight ratio of a therapeutic agent to the biodegradable polymer metal salt may for example be about 1:100000 to about 1:1, for example about 1:20000 to about 1:500 or about 1:10000 to about 1:500. Next, the organic solvent solution containing the biodegradable polymer metal salt and therapeutic agent can be poured into an aqueous phase to prepare an oil/water emulsion. The solvent in the oil phase can then evaporated off to provide microspheres. Finally, these microspheres can then be recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In some cases, a medium comprising a microsphere or microcapsule capable of delivering a controlled release of a therapeutic agent for a duration of approximately between 1 day and 6 months can be used. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

In some cases, a microparticle comprising a gelatin, or other polymeric cation having a similar charge density to gelatin (i.e., poly-L-lysine) can be is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent can be directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium can also be empirically determined by the final density of glutaraldehyde-spacer crosslinks.

### Donor Polynucleotide (Donor Template)

In some cases, a subject composition or method may include a donor polynucleotide. For example, in applications in which it is desirable to insert a polynucleotide sequence into the genome where a target sequence is cleaved, a donor polynucleotide (a nucleic acid comprising a donor sequence) can also be provided to the cell. By a “donor sequence” or “donor polynucleotide” or “donor template” it is meant a nucleic acid sequence to be inserted at the site targeted by the CRISPR complex (e.g., after dsDNA cleavage, after nicking a target DNA, after dual nicking a target DNA, and the like). In some cases, the donor sequence is provided to the cell as single-stranded DNA. In some cases, the donor template is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. A donor template can be introduced into a cell as part of a vector molecule having additional sequences such as, for example, replication origins, promoters and genes encoding antibiotic resistance. Moreover, donor template can be introduced as naked nucleic acid, as nucleic acid complexed with an agent such as a liposome or poloxamer, or can be delivered by viruses (e.g., adenovirus, AAV).

### Kits

The present disclosure provides kits. In some cases, a subject kit includes one or more components described herein—in any combination. For example, in some cases a subject kit includes a nucleic acid of the present disclosure (e.g., a subject nucleic acid system having first and second nucleic acid sequences that encode an Acr protein and a Cas protein, where the first, the second, or both sequences are operably linked to a translational control element). In some cases, a kit can further include reagents for measuring on-target and off-target nucleic acid targeting events. In some cases, a kit includes a donor polynucleotide. In some cases, a kit includes a collection of vectors with various combinations of translational control elements (e.g., see those described herein as examples).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below as Sets A-D. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Set A (2A peptide)
1. A system comprising one or more nucleic acids, wherein the one or more nucleic acids comprise:
(a) a first nucleotide sequence encoding a Cas effector protein;
(b) a second nucleotide sequence encoding an anti-CRISPR protein (Acr protein), wherein the Acr protein is an inhibitor of the Cas effector protein; and
(c) a translational control element that regulates translation of the Cas effector protein or the Acr protein, thereby modulating activity of the Cas effector protein.
2. The system of 1, wherein the activity of the Cas effector protein that is modulated is nucleic acid cleavage.
3. The system of 1, wherein the activity of the Cas effector protein that is modulated is nucleic acid binding, base editing, transcription modulation, nucleic acid modification, protein modification, and/or or histone modification.
4. The system of any one of 1-3, wherein the Acr protein modulates the level or rate of on-target and/or off-target activity of the Cas effector protein.
5. The system of 4, wherein the amount of on-target activity of the Cas effector protein is increased by the system as compared with a similar system lacking (c).
6. The system of 4, wherein the amount of off-target activity of the Cas effector protein is decreased by the system as compared with a similar system lacking (c).
7. The system of 4, wherein the ratio of on-target activity to off-target activity of the Cas effector protein is increased by the system as compared with a similar system lacking (c).
8. The system of any one of 1-7, wherein the system comprises a nucleic acid that comprises both the first and the second nucleotide sequences.
9. The system of 8, wherein the nucleic acid that comprises both the first and the second nucleotide sequences is a viral vector.
10. The system of any one of 1-9, wherein the system comprises a nucleic acid that comprises both the first and that second nucleotide sequences as part of the same expression cassette, and wherein the translational control element is positioned upstream of the second nucleotide sequence.
11. The system of any one of 1-10, wherein the translational control element is a sequence that links the first and second nucleotide sequences to one another such that the Cas nuclease and the Acr protein are encoded by a polycistronic sequence.
12. The system of any one of 1-11, wherein the translational control element encodes one or more 2A peptides.
13. The system of 12, wherein the first and second nucleotide sequences are positioned in tandem and the translational control element is positioned between them.
14. The system of 13, wherein the first nucleotide sequence is positioned 5′ of the second nucleotide sequence.
15. The system of 13, wherein the second nucleotide sequence is positioned 5′ of the first nucleotide sequence.
16. The system of any one of 12-15, wherein the one or more 2A peptides are selected from the group consisting of: P2A, F2A, E2A, T2A, and any combination thereof.
17. The system of any one of 12-15, wherein at least one of the one or more 2A peptides comprise an amino acid sequence set forth in any one of SEQ ID Nos. 133-138.
18. The system of any one of 12-17, wherein the first and second nucleotide sequences are operably linked to different promoters.
19. The system of 18, wherein a spacer encoding sequence is positioned 5′ of the first nucleotide sequence and is operably linked to the same promoter, and wherein the translational control element is positioned between the spacer encoding sequence and the first nucleotide sequence.
20. The system of 18, wherein a spacer encoding sequence is positioned 5′ of the second nucleotide sequence and is operably linked to the same promoter, and wherein the translational control element is positioned between the spacer encoding sequence and the second nucleotide sequence.
21. The system of any one of 12-20, wherein the translational control element encodes 2 or more 2A peptides in tandem.
22. The system of any one of 12-20, wherein the translational control element encodes 2, 3, 4, or 5 2A peptides in tandem.
23. The system of any one of 1-22, wherein the first and/or second nucleotide sequences are operably linked to a promoter selected from the group consisting of: CMV, miniCMV, EFS, chicken β-actin (CBA), human β-actin, herpes simplex virus thymidine kinase, hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).
24. The system of any one of 1-22, wherein the first and second nucleotide sequences are both operably linked to the same promoter and the promoter is selected from the group consisting of: CMV, miniCMV, EFS, chicken β-actin (CBA), human β-actin, herpes simplex virus thymidine kinase, hybrid promoter CBh, synthetic promoter CAG, human elongation factor-1 alpha (EF1a) EF1a short (EFS), human phosphoglycerate kinase (PG K), mammalian ubiquitin C (UBC), and simian virus 40 (SV40).
25. The system of any one of 1-24, wherein the system is configured such that the ratio of the Cas effector protein to the Acr protein, once said system is introduced into a eukaryotic cell, is between 1:10 to 10:1.
26. The system of any one of 1-25, wherein the Cas effector protein is selected from the group consisting of a Cas3, a Cas9, a Cas12, and a Cas13.
27. The system of any one of 1-26, wherein the Acr protein is selected from Table 1 or Table 2.
28. The system of 27, wherein the Cas effector protein comprises an S. pyogenes Cas9 and the Acr protein comprises AcrIIA2.
29. The system of 28, wherein the AcrIIA2 comprises an amino acid replacement at one or more positions selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122, and K123.
30. The system of 29, wherein the amino acid replacement at the one or more positions is alanine.
31. The system of 27, wherein the Cas effector protein comprises an S. pyogenes Cas9 and the Acr protein comprises AcrIIA4.
32. The system of 31, wherein the AcrIIA4 comprises an amino acid replacement at one or more positions selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87.
33. The system of 32, wherein the replacement at the one or more positions is alanine or arginine.
34. The system of 32, wherein the AcrIIA4 comprises one or more amino acid replacements selected from the group consisting of D14A, G38A, and N39A.
35. The system of 32, wherein the AcrIIA4 comprises the amino replacement N39A or the amino acid replacements D14A and G38A.
36. The system of 8, wherein the nucleic acid that comprises both the first and the second nucleotide sequences comprises an origin of replication.
37. The system of 8, wherein the nucleic acid that comprises both the first and the second nucleotide sequences is an integrative vector.
38. The system of any one of 1-37, further comprising a CRISPR/Cas guide RNA.
39. A method of controlling the editing activity of a Cas effector protein comprising: contacting a target nucleic acid with the system of 38;
whereby the Cas effector protein mediates one or more edits to the target nucleic acid.
40. The method of 39, wherein the level of the Acr protein in the host cell is reduced as compared to a cell provided with a comparable system lacking the translational control element.
41. The method of 39 or 40, wherein the off-target rate of the Cas effector protein is reduced as compared to a cell provided with a comparable system lacking the translational control element.
42. The method of 39 or 40, wherein the off-target rate of the Cas effector protein is reduced as compared to a cell expressing the Cas effector protein but lacking expression of the Acr protein.
43. The method of 39 or 40, wherein the ratio of on-target editing to off-target editing of the Cas effector protein is increased as compared to a cell provided with a comparable system lacking the translational control element.
44. The method of 39 or 40, wherein the ratio of on-target editing to off-target editing of the Cas effector protein is increased as compared to a cell expressing the Cas effector protein but lacking expression of the Acr protein.
45. The method of any one of 39-44, wherein said contacting comprises introducing the system of 38 into a host cell that comprises the target nucleic acid
46. The method of any one of 39-44, wherein the method is carried out in a cell-free in vitro environment.

### Set B (IRES)

1. A system comprising one or more nucleic acids, wherein the one or more nucleic acids comprise:
(a) a first nucleotide sequence encoding a Cas effector protein;
(b) a second nucleotide sequence encoding an anti-CRISPR protein (Acr protein), wherein the Acr protein is an inhibitor of the Cas effector protein; and
(c) a translational control element that regulates translation of the Cas effector protein or the Acr protein, thereby modulating activity of the Cas effector protein.
2. The system of 1, wherein the activity of the Cas effector protein that is modulated is nucleic acid cleavage.
3. The system of 1, wherein the activity of the Cas effector protein that is modulated is nucleic acid binding, base editing, transcription modulation, nucleic acid modification, protein modification, and/or or histone modification.
4. The system of any one of 1-3, wherein the Acr protein modulates the level or rate of on-target and/or off-target activity of the Cas effector protein.
5. The system of 4, wherein the amount of on-target activity of the Cas effector protein is increased by the system as compared with a similar system lacking (c).
6. The system of 4, wherein the amount of off-target activity of the Cas effector protein is decreased by the system as compared with a similar system lacking (c).
7. The system of 4, wherein the ratio of on-target activity to off-target activity of the Cas effector protein is increased by the system as compared with a similar system lacking (c).
8. The system of any one of 1-7, wherein the system comprises a nucleic acid that comprises both the first and the second nucleotide sequences.
9. The system of 8, wherein the nucleic acid that comprises both the first and the second nucleotide sequences is a viral vector.
10. The system of any one of 1-9, wherein the system comprises a nucleic acid that comprises both the first and that second nucleotide sequences as part of the same expression cassette, and wherein the translational control element is positioned upstream of the second nucleotide sequence.
11. The system of any one of 1-10, wherein the translational control element is a sequence that links the first and second nucleotide sequences to one another such that the Cas nuclease and the Acr protein are encoded by a polycistronic sequence.
12. The system of any one of 1-11, wherein the translational control element is an IRES sequence.
13. The system of 12, wherein the first and second nucleotide sequences are positioned in tandem and the translational control element is positioned between them.
14. The system of 13, wherein the first nucleotide sequence is positioned 5′ of the second nucleotide sequence.
15. The system of 13, wherein the first nucleotide sequence is positioned 3′ of the second nucleotide sequence.
16. The system of any one of 12-15, wherein the first and second nucleotide sequences are operably linked to different promoters.
17. The system of 16, wherein the translational control element is positioned 5′ of the first nucleotide sequence and is operably linked to the same promoter.
18. The system of 16, wherein the translational control element is positioned 5′ of the second nucleotide sequence and is operably linked to the same promoter
19. The system of 16, wherein a spacer encoding sequence is positioned 5′ of the first nucleotide sequence and is operably linked to the same promoter, and wherein the translational control element is positioned between the spacer encoding sequence and the first nucleotide sequence.
20. The system of 16, wherein a spacer encoding sequence is positioned 5′ of the second nucleotide sequence and is operably linked to the same promoter, and wherein the translational control element is positioned between the spacer encoding sequence and the second nucleotide sequence.
21. The system of any one of 12-20, wherein the IRES sequence is selected from the group consisting of: EMCV, BIP, CAT-1, c-myc, HCV, VCIP, Apaf-1, mEMCV-1, mEMCV-2, HRV, NRF, FGF-1, KMI1, KM12, (GAAA)16, (PPT19)4, EMCV mutant 5, EMCV mutant 10, EMCV mutant 15, and EMCV mutant 21.
22. The system of any one of 12-21, wherein the IRES comprises the sequence set forth in any one of SEQ ID Nos. 139-159.
23. The system of any one of 1-22, wherein the first and/or second nucleotide sequences are operably linked to a promoter selected from the group consisting of: CMV, miniCMV, EFS, chicken β-actin (CBA), human β-actin, herpes simplex virus thymidine kinase, hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).
24. The system of any one of 1-22, wherein the first and second nucleotide sequences are both operably linked to the same promoter and the promoter is selected from the group consisting of: CMV, miniCMV, EFS, chicken β-actin (CBA), human β-actin, herpes simplex virus thymidine kinase, hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).
25. The system of any one of 1-24, wherein the system is configured such that the ratio of the Cas effector protein to the Acr protein, once said system is introduced into a eukaryotic cell, is between 1:10 to 10:1.
26. The system of any one of 1-25, wherein the Cas effector protein is selected from the group consisting of a Cas3, a Cas9, a Cas12, and a Cas13.
27. The system of any one of 1-26, wherein the Acr protein is selected from Table 1 or Table 2.
28. The system of 27, wherein the Cas effector protein comprises an S. pyogenes Cas9 and the Acr protein comprises AcrIIA2.
29. The system of 28, wherein the AcrIIA2 comprises an amino acid replacement at one or more positions selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122, and K123.
30. The system of 29, wherein the amino acid replacement at the one or more positions is alanine.
31. The system of 27, wherein the Cas effector protein comprises an S. pyogenes Cas9 and the Acr protein comprises AcrIIA4.
32. The system of 31, wherein the AcrIIA4 comprises an amino acid replacement at one or more positions selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87.
33. The system of 32, wherein the replacement at the one or more positions is alanine or arginine.
34. The system of 32, wherein the AcrIIA4 comprises one or more amino acid replacements selected from the group consisting of D14A, G38A, and N39A.
35. The system of 32, wherein the AcrIIA4 comprises the amino replacement N39A or the amino acid replacements D14A and G38A.
36. The system of 8, wherein the nucleic acid that comprises both the first and the second nucleotide sequences comprises an origin of replication.
37. The system of 8, wherein the nucleic acid that comprises both the first and the second nucleotide sequences is an integrative vector.
38. The system of any one of 1-37, further comprising a CRISPR/Cas guide RNA.
39. A method of controlling the editing activity of a Cas effector protein comprising: contacting a target nucleic acid with the system of 38;
whereby the Cas effector protein mediates one or more edits to the target nucleic acid.
40. The method of 39, wherein the level of the Acr protein in the host cell is reduced as compared to a cell provided with a comparable system lacking the translational control element.
41. The method of 39 or 40, wherein the off-target rate of the Cas effector protein is reduced as compared to a cell provided with a comparable system lacking the translational control element.
42. The method of 39 or 40, wherein the off-target rate of the Cas effector protein is reduced as compared to a cell expressing the Cas effector protein but lacking expression of the Acr protein.
43. The method of 39 or 40, wherein the ratio of on-target editing to off-target editing of the Cas effector protein is increased as compared to a cell provided with a comparable system lacking the translational control element.
44. The method of 39 or 40, wherein the ratio of on-target editing to off-target editing of the Cas effector protein is increased as compared to a cell expressing the Cas effector protein but lacking expression of the Acr protein.
45. The method of any one of 39-44, wherein said contacting comprises introducing the system of 38 into a host cell that comprises the target nucleic acid 46. The method of any one of 39-44, wherein the method is carried out in a cell-free in vitro environment

### Set C (Start Codon)

1. A system comprising one or more nucleic acids, wherein the one or more nucleic acids comprise:
(a) a first nucleotide sequence encoding a Cas effector protein;
(b) a second nucleotide sequence encoding an anti-CRISPR protein (Acr protein), wherein the Acr protein is an inhibitor of the Cas effector protein; and
(c) a translational control element that regulates translation of the Cas effector protein or the Acr protein, thereby modulating activity of the Cas effector protein.
2. The system of 1, wherein the activity of the Cas effector protein that is modulated is nucleic acid cleavage.
3. The system of 1, wherein the activity of the Cas effector protein that is modulated is nucleic acid binding, base editing, transcription modulation, nucleic acid modification, protein modification, and/or or histone modification.
4. The system of any one of 1-3, wherein the Acr protein modulates the level or rate of on-target and/or off-target activity of the Cas effector protein.
5. The system of 4, wherein the amount of on-target activity of the Cas effector protein is increased by the system as compared with a similar system lacking (c).
6. The system of 4, wherein the amount of off-target activity of the Cas effector protein is decreased by the system as compared with a similar system lacking (c).
7. The system of 4, wherein the ratio of on-target activity to off-target activity of the Cas effector protein is increased by the system as compared with a similar system lacking (c).
8. The system of any one of 1-7, wherein the system comprises a nucleic acid that comprises both the first and the second nucleotide sequences.
9. The system of 8, wherein the nucleic acid that comprises both the first and the second nucleotide sequences is a viral vector.
10. The system of any one of 1-9, wherein the system comprises a nucleic acid that comprises both the first nucleotide sequence operably linked to a first promoter, and the second nucleotide sequence operably linked to a second promoter.
11. The system of 10, wherein the translational control element is positioned upstream of the second nucleotide sequence.
12. The system of 10 or 11, wherein the first and second promoters are different from each other.
13. The system of any one of 1-12, wherein the translational control element comprises a non-AUG start codon that is in frame with, and 5′ of, the second nucleotide sequence.
14. The system of 13, wherein the second nucleotide sequence does not comprise a native in-frame AUG start codon.
15. The system of any one of 1-12, wherein the translational control element comprises a non-AUG start codon that is in frame with, and 5′ of, the first nucleotide sequence.
16. The system of 15, wherein the first nucleotide sequence does not comprise a native in-frame AUG start codon.
17. The system of any one of 13-16, wherein the non-AUG start codon comprises any one of CUG, GUG, ACG, AUA or UUG.
18. The system of any one of 1-17, wherein the first nucleotide sequence is positioned 5′ of the second nucleotide sequence.
19. The system of any one of 1-17, wherein the first nucleotide sequence is positioned 3′ of the second nucleotide sequence.
20. The system of any one of 1-19, wherein the first and/or second nucleotide sequences are operably linked to a promoter selected from the group consisting of: CMV, miniCMV, EFS, chicken β-actin (CBA), human β-actin, herpes simplex virus thymidine kinase, hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).
21. The system of any one of 1-19, wherein the first and second nucleotide sequences are both operably linked to the same promoter and the promoter is selected from the group consisting of: CMV, miniCMV, EFS, chicken β-actin (CBA), human β-actin, herpes simplex virus thymidine kinase, hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).
22. The system of any one of 1-21, wherein the system is configured such that the ratio of the Cas effector protein to the Acr protein, once said system is introduced into a eukaryotic cell, is between 1:10 to 10:1.
23. The system of any one of 1-22, wherein the Cas effector protein is selected from the group consisting of a Cas3, a Cas9, a Cas12, and a Cas13.
24. The system of any one of 1-23, wherein the Acr protein is selected from Table 1 or Table 2.
25. The system of 24, wherein the Cas effector protein comprises an S. pyogenes Cas9 and the Acr protein comprises AcrIIA2.
26. The system of 25, wherein the AcrIIA2 comprises an amino acid replacement at one or more positions selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122, and K123.
27. The system of 26, wherein the amino acid replacement at the one or more positions is alanine.
28. The system of 24, wherein the Cas effector protein comprises an S. pyogenes Cas9 and the Acr protein comprises AcrIIA4.
29. The system of 28, wherein the AcrIIA4 comprises an amino acid replacement at one or more positions selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87.
30. The system of 29, wherein the replacement at the one or more positions is alanine or arginine.
31. The system of 29, wherein the AcrIIA4 comprises one or more amino acid replacements selected from the group consisting of D14A, G38A, and N39A.
32. The system of 29, wherein the AcrIIA4 comprises the amino replacement N39A or the amino acid replacements D14A and G38A.
33. The system of 5, wherein the nucleic acid that comprises both the first and the second nucleotide sequences comprises an origin of replication.
34. The system of 5, wherein the nucleic acid that comprises both the first and the second nucleotide sequences is an integrative vector.
35. The system of any one of 1-34, further comprising a CRISPR/Cas guide RNA.
36. A method of controlling the editing activity of a Cas effector protein comprising:
   contacting a target nucleic acid with the system of 35;
   whereby the Cas effector protein mediates one or more edits to the target nucleic acid.
37. The method of 36, wherein the level of the Acr protein in the host cell is reduced as compared to a cell provided with a comparable system lacking the translational control element.
38. The method of 36 or 37, wherein the off-target rate of the Cas effector protein is reduced as compared to a cell provided with a comparable system lacking the translational control element.
39. The method of 36 or 37, wherein the off-target rate of the Cas effector protein is reduced as compared to a cell expressing the Cas effector protein but lacking expression of the Acr protein.
40. The method of 36 or 37, wherein the ratio of on-target editing to off-target editing of the Cas effector protein is increased as compared to a cell provided with a comparable system lacking the translational control element.
41. The method of 36 or 37, wherein the ratio of on-target editing to off-target editing of the Cas effector protein is increased as compared to a cell expressing the Cas effector protein but lacking expression of the Acr protein.
42. The method of any one of 36-41, wherein said contacting comprises introducing the system of 38 or 39 into a host cell that comprises the target nucleic acid
43. The method of any one of 36-41, wherein the method is carried out in a cell-free in vitro environment.

### Set D (Combo—PCT Claims)

1. A system comprising one or more nucleic acids, wherein the one or more nucleic acids comprise:
   (a) a first nucleotide sequence encoding a Cas effector protein;
   (b) a second nucleotide sequence encoding an anti-CRISPR protein (Acr protein), wherein the Acr protein is an inhibitor of the Cas effector protein; and
   (c) a translational control element that regulates translation of the Cas effector protein or the Acr protein, thereby modulating activity of the Cas effector protein.
2. The system of 1, wherein the activity of the Cas effector protein that is modulated is nucleic acid cleavage.
3. The system of 1, wherein the activity of the Cas effector protein that is modulated is nucleic acid binding, base editing, transcription modulation, nucleic acid modification, protein modification, and/or or histone modification.
4. The system of any one of 1-3, wherein the Acr protein modulates the level or rate of on-target and/or off-target activity of the Cas effector protein.
5. The system of 4, wherein the amount of on-target activity of the Cas effector protein is increased by the system as compared with a similar system lacking the translational control element.
6. The system of 4, wherein the amount of off-target activity of the Cas effector protein is decreased by the system as compared with a similar system lacking the translational control element.
7. The system of 4, wherein the ratio of on-target activity to off-target activity of the Cas effector protein is increased by the system as compared with a similar system lacking the translational control element.
8. The system of any one of 1-7, wherein at least one of said one or more nucleic acids is a nucleic acid vector that comprises the first nucleic sequence and the second nucleic sequence.
9. The system of 8, wherein the nucleic acid vector is a viral vector.
10. The system of 8, wherein the nucleic acid vector comprises an origin of replication.
11. The system of 8, wherein the nucleic acid vector is an integrative vector.
12. The system of any one of 1-11, further comprising a CRISPR/Cas guide RNA or a nucleic acid that encodes the CRISPR/Cas guide RNA.
13. The system of any one of 8-11, wherein the nucleic acid vector encodes a CRISPR/Cas guide RNA.
14. The system of any one of 1-13, wherein at least one of said one or more nucleic acids comprises an expression cassette comprising the first nucleic sequence, the second nucleic sequence, and the translational control element, wherein the translational control element is positioned upstream of the first nucleotide sequence.
15. The system of any one of 1-13, wherein at least one of said one or more nucleic acids comprises an expression cassette comprising the first nucleic sequence, the second nucleic sequence, and the translational control element, wherein the translational control element is positioned upstream of the second nucleotide sequence.
16. The system of any one of 1-13, wherein at least one of said one or more nucleic acids comprises an expression cassette comprising the first nucleic sequence, the second nucleic sequence, and the translational control element, wherein the translational control element is positioned between the first nucleotide sequence and the second nucleotide sequence.
17. The system of 16, wherein the translational control element is a sequence that links the first and second nucleotide sequences to one another such that the Cas nuclease and the Acr protein are encoded by a polycistronic sequence.
18. The system of 16 or 17, wherein the first nucleotide sequence is 5′ to the second nucleotide sequence.
19. The system of 16 or 17, wherein the second nucleotide sequence is 5′ to the first nucleotide sequence.
20. The system of any one of 1-19, wherein the translational control element is an

IRES sequence.
21. The system of 20, wherein the IRES sequence is selected from the group consisting of EMCV, BIP, CAT-1, c-myc, HCV, VCIP, Apaf-1, mEMCV-1, mEMCV-2, HRV, NRF, FGF-1, KMI1, KMI2, (GAAA)16, (PPT19)4, EMCV mutant 5, EMCV mutant 10, EMCV mutant 15, and EMCV mutant 21, and any combination thereof.
22. The system of 20 or 21, wherein the IRES sequence comprises the sequence set forth in any one of SEQ ID Nos. 139-159.
23. The system of any one of 1-19, wherein the translational control element encodes one or more 2A peptides.
24. The system of 23, wherein the one or more 2A peptides are selected from the group consisting of: P2A, F2A, E2A, T2A, and any combination thereof.
25. The system of 23 or 24, wherein at least one of the one or more 2A peptides comprises an amino acid sequence set forth in any one of SEQ ID Nos. 133-138.
26. The system of any one of 23-25, wherein the translational control element encodes two or more 2A peptides in tandem.
27. The system of any one of 23-25, wherein the translational control element encodes 2, 3, 4, or 5 2A peptides in tandem.
28. The system of any one of 1-15, 18 or 19, wherein the translational control element is a non-AUG start codon.
29. The system of 28, wherein the non-AUG start codon is at the 5′ end and in-frame with the first nucleotide sequence.
30. The system of 29, wherein the first nucleotide sequence does not comprise a native in-frame AUG start codon.
31. The system of 28, wherein the non-AUG start codon is at the 5′ end and in-frame with the second nucleotide sequence.
32. The system of 31, wherein the second nucleotide sequence does not comprise a native in-frame AUG start codon.
33. The system of any one of 28-32, wherein the non-AUG start codon comprises any one of CUG, GUG, ACG, AUA or UUG.
34. The system of any one of 1-33, wherein a promoter is operably linked to the first nucleotide sequence.
35. The system of any one of 1-33, wherein a promoter is operably linked to the second nucleotide sequence.
36. The system of any one of 1-33, wherein a first promoter is operably linked to the first nucleotide sequence and a second promoter is operably linked to the second nucleotide sequence.
37. The system of 36, wherein a spacer encoding sequence is positioned 5′ of the first nucleotide sequence and is operably linked to the first promoter, and wherein the translational control element is positioned between the spacer encoding sequence and the first nucleotide sequence.
38. The system of 36, wherein a spacer encoding sequence is positioned 5′ of the second nucleotide sequence and is operably linked to the second promoter, and wherein the translational control element is positioned between the spacer encoding sequence and the second nucleotide sequence.
39. The system of any one of 1-27, wherein a promoter is operably linked to the first nucleotide sequence, and the first nucleotide sequence is 5′ to the translational control element and the second nucleotide sequence.
40. The system of any one of 1-27, wherein a promoter is operably linked to the second nucleotide sequence, and the second nucleotide sequence is 5′ to the translational control element and the first nucleotide sequence.
41. The system of any one of 34-35 and 39-40, wherein the promoter is selected from the group consisting of CMV, miniCMV, EFS, chicken β-actin (CBA), human β-actin, herpes simplex virus thymidine kinase hybrid promoter CBh, synthetic promoter CAG, human elongation factor-1 alpha (EF1a) EF1a short (EFS), human phosphoglycerate kinase (PG K), mammalian ubiquitin C (UBC), and simian virus 40 (SV40).
42. The system of any one of 36-38, wherein the first promoter and/or the second promoter is selected from the group consisting of CMV, miniCMV, EFS, chicken β-actin (CBA), human β-actin, herpes simplex virus thymidine kinase hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).
43. The system of any one of 1-42, wherein the Cas effector protein is selected from the group consisting of a Cas3, a Cas9, a Cas12, and a Cas13.
44. The system of 43, wherein the Cas effector protein comprises an amino acid sequence having 70% or more identity with the sequence set forth in any one of SEQ ID Nos. 83-86.
45. The system of any one of 1-44, wherein the Acr protein is selected from Table 1 or Table 2.
46. The system of any one of 1-44, wherein the Acr protein comprises an amino acid sequence having 70% or more identity with the sequence set forth in any one of SEQ ID Nos. 1-82 and 161.
47. The system of any one of 1-44, wherein the Cas effector protein comprises an S. pyogenes Cas9.
48. The system of 47, wherein the Acr protein is an AcrIIA2 protein.
49. The system of 48, wherein the AcrIIA2 protein comprises an amino acid replacement at one or more positions selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122, and K123.
50. The system of 49, wherein the amino acid replacement at the one or more positions is alanine.
51. The system of 47, wherein the Acr protein comprises an AcrIIA4 protein.
52. The system of 51, wherein the AcrIIA4 protein comprises an amino acid replacement at one or more positions selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87.
53. The system of 52, wherein the replacement at the one or more positions is alanine or arginine.
54. The system of 52, wherein the AcrIIA4 protein comprises one or more amino acid replacements selected from the group consisting of D14A, G38A, and N39A.
55. The system of 52, wherein the AcrIIA4 protein comprises the amino replacement N39A or the amino acid replacements D14A and G38A.
56. The system of 47, wherein the Acr protein is selected from the group consisting of Acx105, Acx137, Acx, 153, Acx162, and Acx164.
57. A cell comprising the system according to any one of 1-56.
58. The cell of 57, wherein the cell is a mammalian cell or a microorganism.
59. The cell of 57, wherein the cell is a human cell.
60. A method of controlling the editing activity of a Cas effector protein comprising: contacting a target nucleic acid with the system of any one of 1-56, whereby the Cas effector protein mediates one or more edits to a target sequence of the target nucleic acid.
61. The method of 60, further comprising measuring the efficacy, level or amount of edits to the target sequence.
62. The method of 60, further comprising detecting or identifying one or more edits to the target sequence.
63. The method of any one of 60-62, further comprising detecting or identifying one or more edits to a non-target sequence.
64. The method of any one of 60-62, further comprising detecting or identifying one or more edits to a non-target sequence.
65. The method of 64, further comprising measuring the efficacy, level or amount of edits to the non-target sequence.
66. The method of 60, wherein the system provides a ratio of editing the target sequence to editing a non-target sequence is greater than a second ratio of editing the target sequence to editing a non-target sequence provided by the system lacking the Acr protein.
67. The method of 60, wherein the system provides a ratio of editing the target sequence to editing a non-target sequence is greater than a second ratio of editing the target sequence to editing a non-target sequence provided by the system lacking the translational control element.
68. The method of 60, wherein the system provides an efficiency of editing the target sequence that is greater than an efficiency of editing a non-target sequence.
69. The method of 63, wherein the target sequence and the non-target sequence share greater than 90% but less than 100% sequence identity.
70. The method of 63 or 64, wherein the efficiency of editing the target sequence is at least 2x, 4x, 5x, 10x, 12x, 15x, 20x, 25x, 30x, 35x greater than the efficiency of editing a non-target sequence.
71. The method of 63 or 64, wherein the ratio of editing the target sequence to editing the non-target sequence is at least 2, 4, 5, 10, 12, 15, 20, 25, 30, 35 or greater than 35.
72. The method of any one of 60-71, wherein the target nucleic acid is in a cell.
73. The method of 72, wherein the cell is a mammalian cell or a microorganism.
74. The method of 72, wherein the cell is a human cell.
75. The method of any one of 72-74, wherein the contacting step comprises introducing the system into the cell.
76. The method of any one of 60-71, wherein the target nucleic acid is not inside of a cell.
77. The method of 76, wherein the method is an in vitro assay.
78. The method of 77, wherein the in vitro assay is a diagnostic assay.

### VI. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### ACR Sequences for Examples 1A-1C [See, e.g, Table 2)

Acr mutants: Acx153 (SEQ ID NO: 81), Acx162 (SEQ ID NO: 161), Acx164 (SEQ ID NO: 82).

Acr wild type controls: Acx105 (SEQ ID NO: 35), Acx137 (SEQ ID NO: 67)

### Example 1A: Construction of IRES-Regulated Vectors

Expression vectors to deliver Cas nuclease and single gRNA (guide RNA) transiently to the HEK293 cells were constructed with an RNA polymerase III-dependent U6 promoter was used to transcribe the sgRNA and a CMV promoter was used to drive transcription of the mRNA for the Cas, Acr and IRES elements. Different internal ribosome entry site (IRES) sequences were placed between the nuclease sequence and the Acr protein sequence in the vectors. These vectors were constructed as follows.

A first vector was constructed to insert an oligonucleotide corresponding to the target site in the backbone vector pSpCas9(BB)-2A-Puro (PX459) V2.0 (Purchased from Genscript). Phosphorylated and annealed oligos (20 bp sequence corresponding to HBB target site from Cradick et al., 2013-gTGAACGTGGATGAAGTTGG (SEQ ID NO: 132)) were cloned into the BbsI digested PX459 vector. The resulting vector was named pX459_HBB.

pX459 HBB was then modified to have the Acr protein and the IRES element. IRES and Acr protein coding regions were synthesized and added into the sequence 3′ to the nuclease, after the nucleoplasmin (NLS) stop codon. The amino acid sequence for each Acr are provided as follows (see, e.g., Table 2): Acx 105 (SEQ ID NO: 35), Acx 137 (SEQ ID NO: 67), Acx 153 (SEQ ID NO: 81), and Acx 162 (Seq ID NO: 161)

Vectors for SpyCas9 and Acx105 were constructed for each of the five IRES sequences (SEQ ID Nos. 139-143) shown in Table 8a. The relative orientations of the elements in the vectors in shown in FIG. 9. The vectors with Acx137, Acx153 and Acx162 contained the EMCV WT IRES (SEQ ID No. 139). For all of these vectors the promoter driving expression of SpCas9 and Acr was the CMV promoter.

Two vectors with the inversion of SpCas9 and Acx were cloned by using the original orientation (SpCas9-IRES-Acx) for Acx-105 and Acx-162 as templates. These were linearized by PCR amplification. This amplification removed the SpCas9 piece and the Acx-piece of each vector. New SpCas9 and Acx inserts were amplified with overhangs adding overlapping regions with the previously digested vector. Linearized vector and the two inserts were ligated with the use of the NEBuilder cloning kit following the manufacturer's protocol. Table 11 provides sequences associated with four of the generated vectors and Table 12 provides sequences used in the vector construction process.

TABLE 8a
IRES sequences for vector construction

| | | | SEQ ID |
|---|---|---|---|
| Name | Description | Strength | No. |
| EMCV WT IRES | Encephalomyocarditis virus | 100% | 139 |
| | (EMCV) IRES | | |
| EMCV IRESv5 | Variant of Encephalomyocarditis | 45.18% | 140 |
| | virus (EMCV) IRESv5 | | |
| EMCV IRESv10 | Variant of Encephalomyocarditis | 29.45% | 141 |
| | virus (EMCV) IRESv10 | | |
| EMCV IRESv15 | Variant of Encephalomyocarditis | 3.23% | 142 |
| | virus (EMCV) IRESv15 | | |
| EMCV IRESv21 | Variant of Encephalomyocarditis | 0.58% | 143 |
| | virus (EMCV) IRESv21 | | |

TABLE 11
sequences associated with four
of the generated vectors

| | | | Plasmid | |
|---|---|---|---|---|
| Nuclease | IRES | Acr | Sequence | Promoter |
| SpCas9 | EMCV WT | Acx-137 | SEQ ID | CMV |
| | (SEQ ID | | NO: 162 | |
| | NO: 139) | | | |
| SpCas9 | EMCV WT | Acx-153 | SEQ ID | CMV |
| | (SEQ ID | | NO: 163 | |
| | NO: 139) | | | |
| SpCas9 | EMCV WT | Acx-162 | SEQ ID | CMV |
| | (SEQ ID | | NO: 164 | |
| | NO: 139) | | | |
| SpCas9 | EMCV WT | Acx-105 | SEQ ID | CMV |
| | SEQ ID | | NO: 165 | |
| | NO: 139) | | | |

TABLE 12
Sequences used during vector construction

| | Forward | Reverse | |
|---|---|---|---|
| | primer | Primer | Amplicon |
| | (SEQ | (SEQ | (SEQ |
| Cloning step | ID NO) | ID NO) | ID NO) |
| Amplification of Acx-162 | 166 | 171 | 176 |
| Amplification of Acx-105 | 167 | 172 | 177 |
| Linearization of Vector | 168 | 173 | 178 |
| Amplification of SpCas9 | 169 | 174 | 179 |
| Amplification of IRES | 170 | 175 | 180 |

### Example 1B: Construction of 2A-Regulated Vectors (2A Constructs)

Expression vectors to deliver Cas nuclease and single gRNA transiently to HEK293 cells were constructed with a polymerase III-dependent U6 promoter used to transcribe the sgRNA. A CMV promoter was used to drive transcription of the mRNA. Different Self-cleaving peptide (2A) sequences were placed between the nuclease sequence and the Acr protein sequence in the vector. The 2A peptide sequences tested are listed below with their predicted translation efficiency. These vectors were constructed as follows.

A first vector was constructed to insert an oligonucleotide corresponding to the target site in the backbone vector pSpCas9(BB)-2A-Puro (PX459) V2.0 (Purchased from Genscript). Phosphorylated and annealed oligos (20 bp sequence corresponding to HBB target site from Cradick et al., 2013-Gtgaacgtggatgaagttgg (SEQ ID NO: 132)) were cloned into the BbsI digested PX459 vector. The resulting vector was named pX459_HBB.

pX459_HBB was then modified to have the Acr protein and the 2A peptides. The nucleotide sequences encoding the 2A peptides were placed upstream and in-frame of the codon for the first methionine of the Acr protein and this cassette was located 3′ to the nuclease, after the nucleoplasmin (NLS) stop codon. The Acr protein (Acx-105) amino acid sequence that was used was: MNINDLIREIKNKDYTVKLSGTDSNSITQLIIRVNNDGNEYVISESENESIVEKFISAFKNG WNQEYEDEEEFYNDMQTITLKSELN (SEQ ID NO: 35)

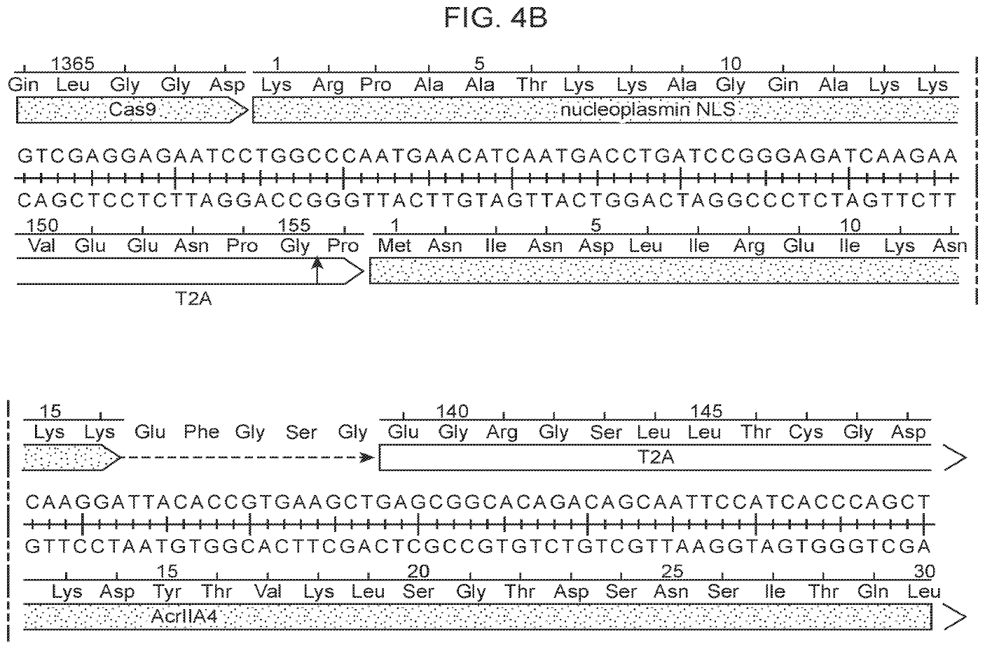

Vectors were constructed with four different 2A peptide combinations shown in Table 7 and the Acr proteins provided in Table 8b. The relative orientations of the elements in the vectors are shown in FIG. 4.

TABLE 7
2A sequences for vector construction

| | | Amino acid | |
|---|---|---|---|
| Name | Description | sequence | Strength |
| T2A | Thosea | EGRGSLLTC | Strong |
| | asigna | GDVEENPGP | |
| | virus 2A | (SEQ ID | |
| | | NO: 136) | |
| F2A | Foot and | VKQTLNFDLLK | Strong |
| | mouth | LAGDVESNPGP | |
| | disease 2A | (SEQ ID | |
| | | NO: 135) | |
| E2A-F2A | Equine | QCTNYALLKLA | Medium |
| | rhinitis A | GDVESNPGPVK | |
| | virus + F2A | QTLNFDLLKLA | |
| | | GDVESNPGP | |
| | | (SEQ ID | |
| | | NO: 137) | |
| T2A-E2A- | Combination | EGRGSLLTCGD | Weak |
| F2A | | VEENPGPQCTN | |
| | | YALLKLAGDVE | |
| | | SNPGPVKQTLN | |
| | | FDLLKLAGDV | |
| | | ESNPGP | |
| | | (SEQ ID | |
| | | NO: 138) | |

TABLE 8b
Acr amino acid sequences

| Acx | Amino Acid sequence |
|---|---|
| 137 | MYEAKERYAKKKMQENTKIDTLTDE |
| | QHDALAQLCAFRHKFHSNKDSLFLS |
| | ESAFSGEFSFEMQSDENSKLREVGL |
| | PTIEWSFYDNSHIPDDSFREWFNFA |
| | NYSELSETIQEQGLELDLDDDETYE |
| | LVYDELYTEAMGEYEELNQDIEKYL |
| | RRIDEEHGTQYCPTGFARLR |
| | (SEQ ID NO: 67) |
| 153 | MNINDLIREIKNKDYTVKLSGTDSN |
| | SITQLIIRVNNDGAEYVISESENES |
| | IVEKFISAFKNGWNQEYEDEEEFYN |
| | DMQTITLKSELN |
| | (SEQ ID NO: 81) |
| 164 | MNINDLIREIKNKAYTVKLSGTDSN |
| | SITQLIIRVNNDANEYVISESENES |
| | IVEKFISAFKNGWNQEYEDEEEFYN |
| | DMQTITLKSELN |
| | (SEQ ID NO: 82) |

### Example 10: Construction of Non-AUG Start Codon Vectors (Start Codon Constructs)

Expression vectors to deliver Cas nuclease and single gRNA transiently to the HEK293 cells were constructed with an RNA polymerase III-dependent U6 promoter to transcribe the sgRNA and a CMV promoter to drive transcription of the mRNA for the Cas. An EF1-alpha promoter was used to transcribe the Acr mRNA and non-canonical start sites were used to mutate the 5′ coding sequence of the Acr. These vectors were constructed as follows.

A first vector was constructed to insert an oligonucleotide corresponding to the target site in the backbone vector pSpCas9(BB)-2A-Puro (PX459) V2.0 (Purchased from Genscript). Phosphorylated and annealed oligos (20 bp sequence corresponding to HBB target site from Cradick et al., 2013-gTGAACGTGGATGAAGTTGG (SEQ ID NO: 132)) were cloned into the BbsI digested PX459 vector. The resulting vector was named pX459_HBB.

pX459_HBB was then modified to place an EF1-alpha promoter 3′ to the SV40 poly(A) signal for the nuclease. The Acr (Acx-105) coding sequence modified with the start-codons (see Table 2) was cloned downstream of the EF1-alpha promoter. Using codon-optimization algorithms, the first 30 nucleotides of the Acr protein was modified to avoid alternative AUGs that could act as a strong downstream start codon:

(Kozak Sequence)
WT:
(GCCACC) ATG AAC ATC AAT
GAC CTG ATC CGG GAG ATC
for CTG:
(GCCACC) cTG AAT ATC AAC
GAT CTC ATT CGG GAG ATC
for GTG:
(GCCACC) gTG AAT ATC AAC
GAT CTC ATT CGG GAG ATC
for ACG:
(GCCACC) AcG AAT ATC AAC
GAT CTC ATT CGG GAG ATC
for ATA:
(GCCACC) ATa AAT ATC AAC
GAT CTC ATT CGG GAG ATC
for TTG:
(GCCACC) tTG AAT ATC AAC
GAT CTC ATT CGG GAG ATC
SEQ ID NOs: 192, 187-191
[from top to bottom: SEQ ID NOs: 192, 187-191]

Vectors were constructed with each of the six different start codon sequences shown in Table 8c. The relative orientations of the elements in the vectors is shown in FIG. 14.

TABLE 8c
Start codons used on the Acr protein for vector construction

| Name | Strength (%) |
|---|---|
| AUG | 100 |
| CUG | 50 |
| GUG | 20 |
| ACG | 10 |
| AUA | 5 |
| UUG | ND |

Similarly, vectors for Acr coding sequences were cloned to make Acx 137 (with AUG start codon) and Acx162 (with either an AUG start codon or a modified CUG start codon). See Table 13.

TABLE 13
Vectors with various start codons for the Acr protein.

| Nuclease | Nuclease | | | | |
|---|---|---|---|---|---|
| (Cas | (Cas | | | | Plasmid |
| effector) | effector) | Acr | Acr start | | sequence |
| promoter | start codon | promoter | codon | Acr | (SEQ ID NO) |
| CMV | ATG | EFS | TTG | 105 | 181 |
| CMV | ATG | EFS | CTG | 105 | 182 |
| CMV | ATG | EFS | ATG | 105 | 183 |
| CMV | ATG | EFS | ATG | 137 | 184 |
| CMV | ATG | EFS | CTG | 162 | 185 |
| CMV | ATG | EFS | ATG | 162 | 186 |

### Example 2: Acr and Cas Expression Levels

Vectors from example 1 were transfected into HEK293 cells as follows. A day prior to transfection, confluent HEK293 cells were seeded into 12 well tissue culture plates using DMEM media (cat #10569010, ThermoFisher) supplemented with 10% FBS Heat-inactivated (cat #: 10100147, ThermoFisher) and 1x penicillin and streptomycin cocktail (cat #15140122, ThermoFisher).

Approximately 1×10⁵ cells were seeded into each well. Turbofect was used as a transfection reagent and the protocol for transfection from the manufacturer (cat #R0532, Thermo Scientific) was followed without modification. After 8 hours of transfection, media was changed for fresh media to optimize cell survival. Transfected cells were incubated for 72h on a tissue culture incubator at 37C° and 5% CO₂.

After 72 h of incubation, samples were washed once with PBS and harvested with 1× trypsin. FBS was used to quench trypsin activity once cells were detached from plates (˜2 minutes). Samples were transferred to 1.5 mL tubes and centrifuged at 300 g for 5 minutes at room temperature. Trypsin+FBS supernatant was removed and cell pellets were put on ice. Samples destined for protein expression assays such as western blot were flash frozen in liquid nitrogen and stored at the −80 C freezer. Samples destined for DNA/editing analysis were resuspended in 100 uL of Quick extract genomic DNA extraction reagent (cat #QE09050, Lucigen).

Protein samples prepared from cell lysates were analyzed by western blotting. Briefly, protein samples were treated with sodium dodecyl sulfate and then separated using gel electrophoresis. The protein gels were transferred to blotting membrane, and after blocking, incubated with primary antibodies against the Cas (Rabbit Anti-SpCas9, cat #65832, cell signaling, dilution 1:1000) and Acr (Mouse Anti-AcrIIA4, cat #C15200248, Diagenode, dilution 1:1000) proteins. Secondary antibodies (Goat Anti-Rabbit IgG (H&L) HRP, cat #A00098, Genscript; dilution 1:1000 and Goat Anti-Mouse IgG (H&L) HRP cat #A00160, Genscript; 1:1000 dilution) were used to visualize the Cas and Acr proteins. To determine the relative protein expression levels and obtain Cas/Acr ratios, protein band intensities were quantified using densitometry by ImageJ software available from the NIH website (https://imagej.nih.gov/ij/download.html). Each band was normalized to an internal reference control such as Actin (Thetm beta Actin Antibody [HRP], mAb, Mouse, dilution 1:1000 cat #A00730-100, Genscript) and HSP90 (HSP90 antibody, dilution 1:1000 cat #4874, Cell Signalling) that allows fluctuations in amount of protein loaded onto each well or different concentrations.

### Example 3: On-Target and Off-Target Editing Measurements

Plasmids were transfected in HEK293T cells using two different delivery methods. In experiment shown in FIG. 15A-15B and Table 9c, plasmids were transfected with the use of Turbofect (Thermo, R0533) following the manufacture's protocol for the 96-well format. Plasmids for all other figures and tables were transfected with TransiT X2 (Mirus) transfection following the manufactures' protocol for the 96-well format.

Samples (resuspended in 100 uL of Quick extract genomic DNA extraction reagent (cat #QE09050, Lucigen) were transferred to PCR tubes and gDNA was extracted by incubating at 65C for 20 minutes followed by 95C for 20 minutes.

Tracking of Indels by Decomposition (TIDE) technique was used (Brinkman et al., 2014). Specific sites were amplified with primers specific to the on-target and known off-target regions.

The on-target sequence was assayed as follows: A 714 nt region of HBB, containing the on-target site was PCR amplified using the following primer sets: For HBB, forward 5′—CGATCCTGAGACTTCCACACTG-3′ (SEQ ID NO: 124) and reverse 5′-CCAATCTACTCCCAGGAGCAGG-3′ (SEQ ID NO: 125).

The PCR reaction was performed by using 100 ng of gDNA and Kapa Hot start high fidelity polymerase (cat #KK2501, Roche) for 30 cycles according to the manufacturer's protocol. The thermocycler setting consisted of one cycle of 95C for 5 minutes, 30 cycles of 98C for 20 s, 68C for 15 s and 72C for 45 s, and 1 cycle of 72C for 1 minute. The PCR products were analyzed on a 2% agarose gel containing SYBR Safe (ApexBio) and samples were sequenced.

For off-target measurements, the following amplifications and sequencing was performed. A 900 nt region of HBD, containing the predicted off-target site, and for HBD, forward 5′-CCCATGTGGAGAGACAAAAGGA-3′ (SEQ ID NO: 126) and reverse 5′-CTTAAACCAACCTGCTCACTGG-3′ (SEQ ID NO: 127) was amplified using 100 ng of gDNA and Kapa Hot start high fidelity polymerase (cat #KK2501, Roche) for 30 cycles according to the manufacturer's protocol. The thermocycler setting consisted of one cycle of 95C for 5 minutes, 30 cycles of 98C for 20 s, 68C for 15 s and 72C for 45 s, and 1 cycle of 72C for 1 minute. The PCR products were analyzed on a 2% agarose gel containing SYBR Safe (ApexBio) and samples were sequenced.

To generate a comparison of on-target and off-target editing, sequencing files from on-target and off-target amplications (above) were sequenced and aligned to the SgRNA sequence. Briefly, the .ab1 files were analyzed with the TIDE software (Brinkman et al., 2014; “htt” followed by “ps://” followed by “tide” followed by “.nki.” followed by “nil”) that aligns the sgRNA sequence to edited and non-edited controls and then decomposes the file using the unedited sample as the background for comparison. This generates in an estimation of the relative abundance and number or insertions and deletions in the edited sample compared to the negative control. Sequencing primers were designed to be 200 bp upstream of the predicting editing site as directed by TIDE software developers. For ON target analysis, an alignment window from nucleotide 20 to 100 and a decomposition window until 350 nt downstream of the predicted editing site was used. For OFF target samples, an alignment window from nucleotide 20 to 100 and a decomposition window until 450 nt downstream of the editing site was used.

### Example 4: Evaluation of IRES Elements

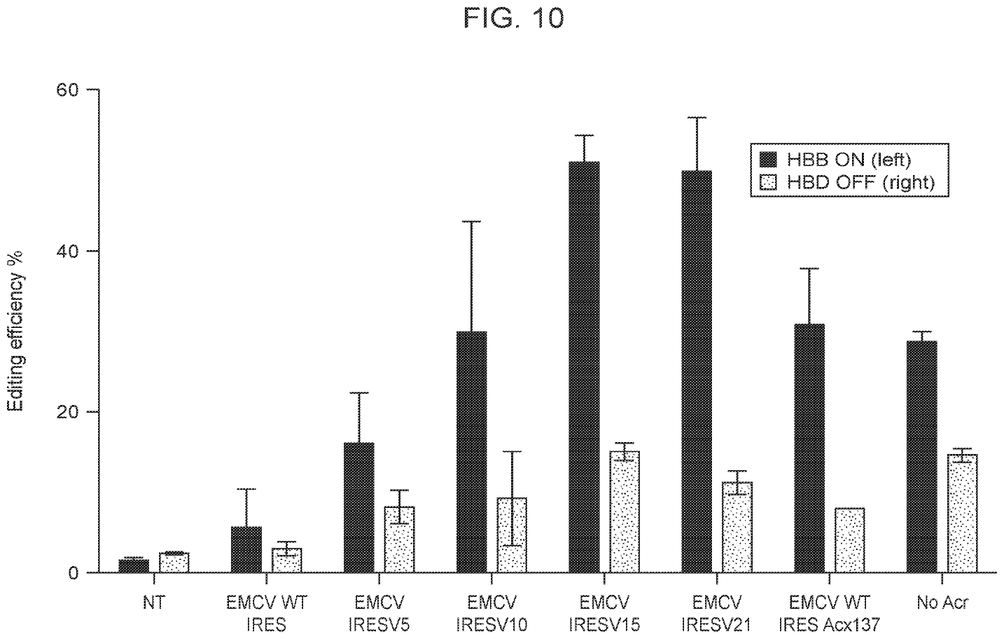

Results are shown in Table 9a (below) and FIG. 10. All variants V5, V10, V15 and V21 are a result of mutations on the 10th, 11th and 12th AUG segments of the IRES element. The use of the wild-type EMCV IRES element provided AcrIIA4 with a strong translation profile, allowing the Acr protein to inhibit SpCas9 activity almost completely. Variants V5 and V10 increasingly weakened translation/expression and an increase in SpCas9 editing capabilities was observed due to less Acr protein being produced. Variants V15 and V21 were responsible for very weak translation/expression and the values of SpCas9 editing were similar to those of no-Acr protein control

TABLE 9a
Indel frequencies calculated by the TIDE tool. R1 and R2 are Replicate
1 and Replicate 2. ON is on-target and OFF is off-target.

| Sample | ON - R1 | ON - R2 | OFF - R1 | OFF - R2 |
|---|---|---|---|---|
| Non-transfected | 1.7 | 1.5 | 2.1 | 2.3 |
| EMCV WT IRES | 1.2 | 9.3 | 2.3 | 3.5 |
| EMCV IRESV5 | 12.5 | 12.1 | 6.6 | 9.5 |
| *EMCV IRESV10 | 426.5 | 3.021.5 | 0.75 | 013.3 |
| | 26.5 | 21.5 | 5 | 13.3 |
| EMCV IRESV15 | 54.3 | 51.6 | 15.8 | 14.2 |
| *EMCV IRESV21 | 48.850 | 43.155 | 12.92 | 13.510.1 |
| | 50 | 55 | 12.2 | 10.1 |
| EMCV WT IRES - | 38.8 | 27.1 | 7.9 | 8 |
| Acx137 | | | | |
| No Acr | 29 | 30 | 15.5 | 14 |

[an asterisk indicates this row had inadvertent typographical errors, and the row immediately below includes the corrected information]

### Example 5: Evaluation of IRES Elements

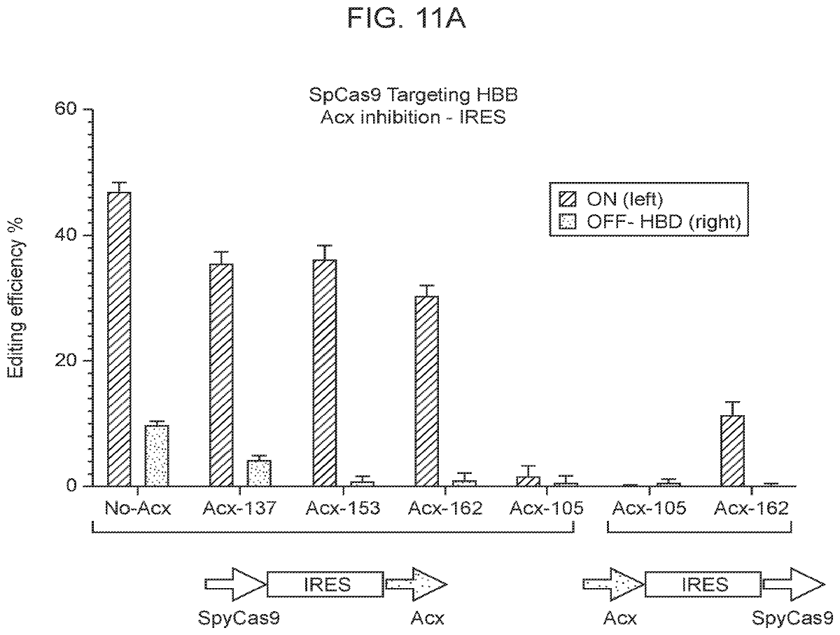

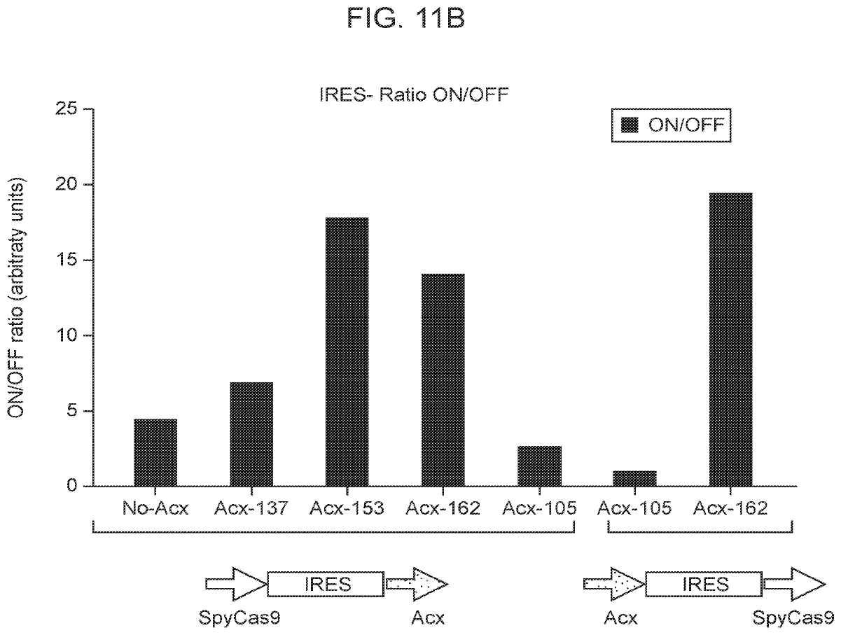

IRES elements were evaluated in combination with SpyCas9 and Acr variants as shown in FIG. 11A and FIG. 11B. Editing efficiency was calculated for on and off target as described in Example 3. Background measurement (sample that did not contain either the Cas nuclease or the Acr) was subtracted and the resulting on and off target measurements are graphed in FIG. 11A. FIG. 11B shows the on/off target ratio.

The IRES element used for these combinations was EMCV WT IRES. In the constructs having an IRES between the nuclease and the Acr, in all cases the off targeting was significantly reduced as compared to the no Acr control (FIG. 11A). For Acr137, Acr153 and Acr162, the ratio of on-target to off-target events was increased above the no Acr control (FIG. 11B). In the reversed orientation (IRES 5′ to the nuclease), the Acrx105 reduced editing significantly. However, Acx162 in this construct design provided a high on-target to off-target ratio

### Example 6A: Evaluation of 2A Peptide Elements

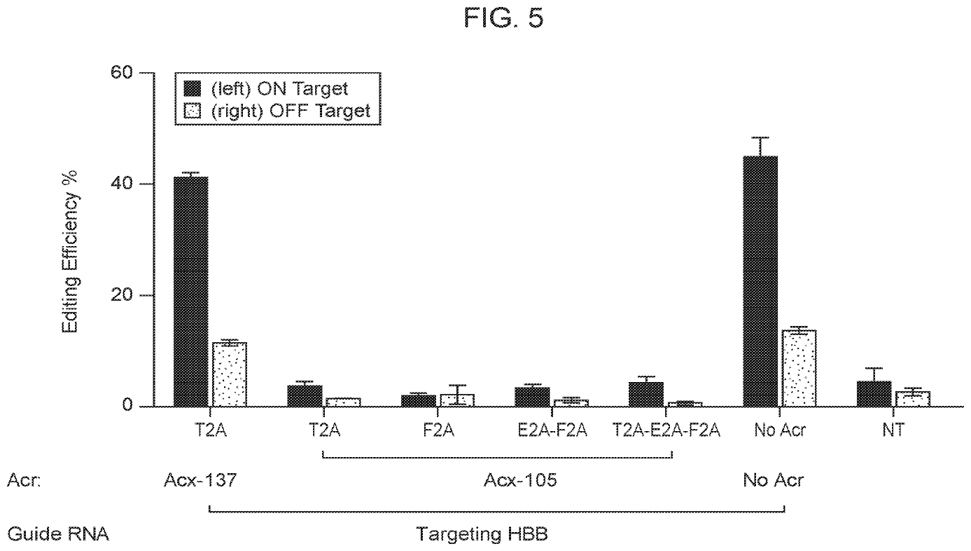

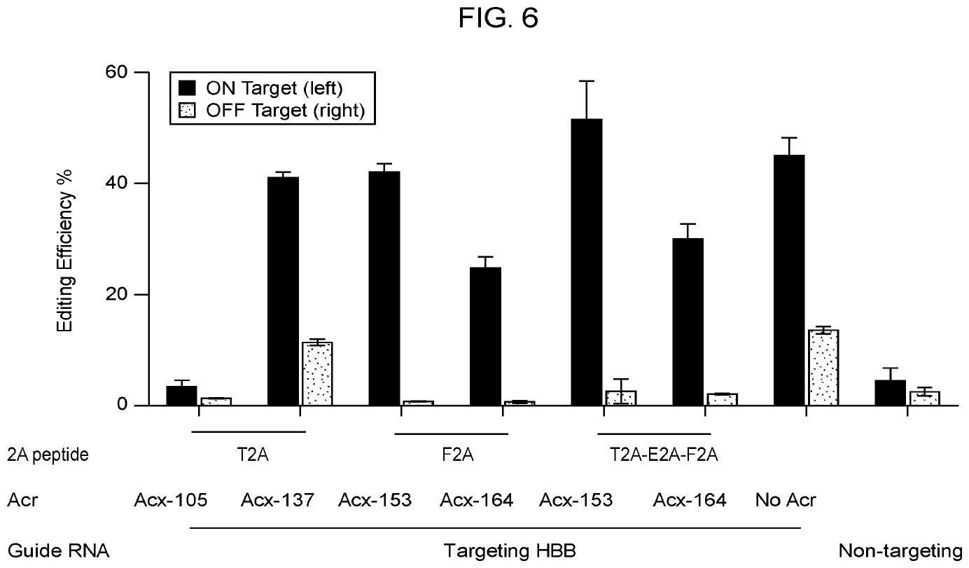

Results are shown in Table 9b and Table 10 and FIG. 5 and FIG. 6. All 2A peptides were efficient at producing Acr protein. The Acx137 is the construct that contains an Acr that does not inhibit SpCas9 and is used here as a control. The use of F2A resulted in the strongest inhibition of SpCas9 editing by Acx-105; followed by the combination E2A-F2A and T2A. The least efficient configuration was the tandem use of T2A-E2A-F2A. Results with the 2A peptides and Acx-153 and Acx-164 are shown in Table 10 and FIG. 6). Similar inhibition profiles (Table 9b) were observed with less inhibition of editing when the tandem 2A configuration was used.

TABLE 9b
Indel frequencies calculated by the TIDE tool. R1 and R2 are Replicate
1 and Replicate 2. ON is on-target and OFF is off-target.

| Sample | ON - R1 | ON - R2 | OFF - R1 | OFF - R2 |
|---|---|---|---|---|
| T2A - Acx -137 | 40.3 | 42.0 | 11.0 | 12.0 |
| T2A - Acx-105 | 3.4 | 4.5 | 1.3 | 1.4 |
| F2A - Acx-105 | 1.2 | 1.8 | 0.8 | 3.2 |
| E2A-F2A - Acx-105 | 2.7 | 3.9 | 1.0 | 0.5 |
| T2A-E2A-F2A - | 4.5 | 3.0 | 0.7 | 0.3 |
| Acx-105 | | | | |
| No Acr | 48.8 | 43.1 | 12.9 | 13.5 |
| NT | 6.1 | 2.6 | 3.0 | 2.0 |

TABLE 10
Indel frequencies calculated by the TIDE tool. R1 and
R2 are Replicate 1 and Replicate 2. ON is on-target
and OFF is off-target for Acx 153 and Acx 164.

| Sample | ON - R1 | ON - R2 | OFF - R1 | OFF - R2 |
|---|---|---|---|---|
| T2A-Acrx-105 | 3.4 | 4.5 | 1.3 | 1.4 |
| T2A-Acx-137 | 40.3 | 42.0 | 11.0 | 12.0 |
| F2A-Acx-153 | 42.7 | 43.1 | 0.7 | 0.9 |
| F2A-Acx-164 | 24.1 | 23.3 | 0.2 | 0.8 |
| T2A-E2A-F2A - | 52.5 | 58.0 | 1.1 | 1.4 |
| Acx-153 | | | | |
| T2A-E2A-F2A - | 28.4 | 33.1 | 2.0 | 2.3 |
| Acx-164 | | | | |
| No Acr | 48.8 | 43.1 | 12.9 | 13.5 |

### Example 6B: Evaluation of 2A Peptide Element

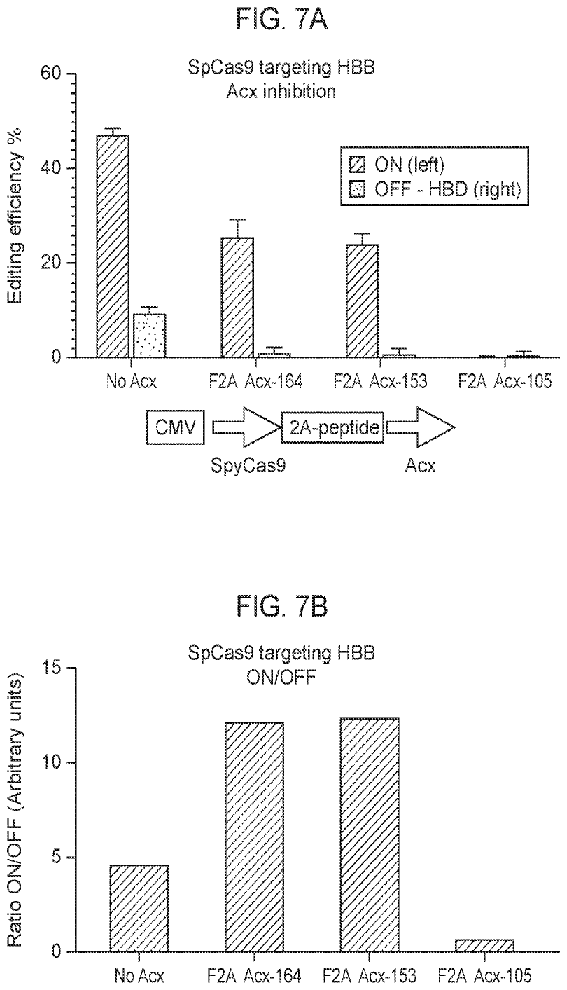

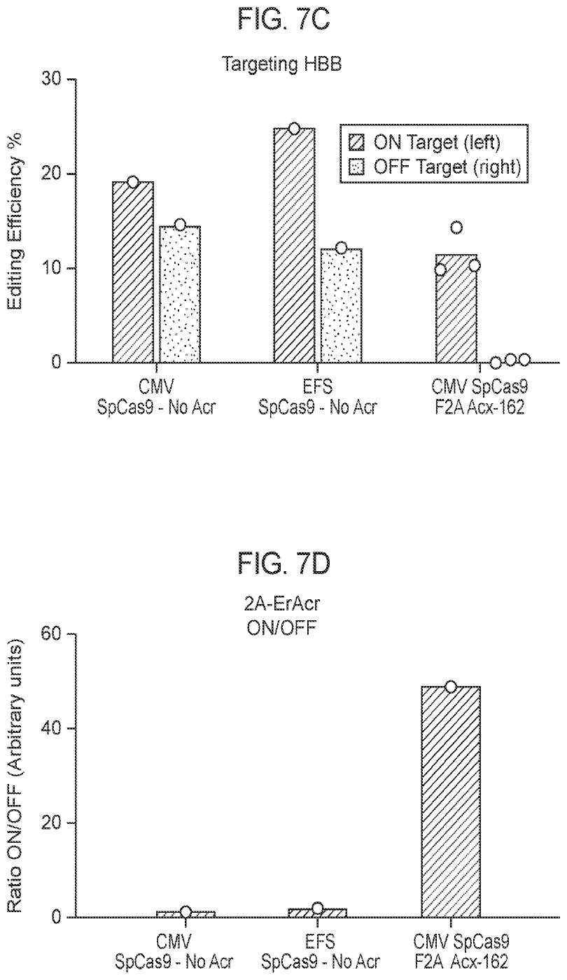

Three Acr elements were chosen to compare with the F2A-Acx105, Acx153 and Acx 164 in the construct orientation show in FIG. 7A, compared with a non Acr control. Acx105 reduced all editing (on and off target), whereas Acx153 and Acx162 in combination with the F2A peptide had a greater effect on off-targeting, with only a moderate reduction in on-targeting editing efficiency (FIG. 7A). In comparison to the no Acr control, both Acx153 and Acx162 in combination with the F2A peptide improved the on-target to off-target ratio (FIG. 7B).

Acx162 was tested in combination with the F2A peptide and compared to the SpCas9 without Acr. As shown in FIG. 7C and FIG. 7D, the Acx162+F2A peptide significantly reduced the off-target editing but had only a small impact on the on-target efficiency.

### Example 7: Evaluation of Non-Canonical Start Sites

Results are shown in Table 9c (below) and FIG. 15. The presence of a canonical start codon, AUG, resulted in strong inhibition by AcrIIA4, reducing SpCas9 editing more than 80%. The use of non-canonical start codons decreased the inhibitory profile and the mutants had a sliding effect, with CUG being the strongest with ˜36% inhibition of ON target editing and ˜50% inhibition of OFF target editing. GUG, UUG and ACG all had very weak inhibitory profiles, with editing percentages similar to no Acr control. Numbers for indel frequencies are shown in Table 9c.

TABLE 9c
Indel frequencies of Non-AUG start codons

| Sample | ON - R1 | ON - R2 | OFF - R1 | OFF - R2 |
|---|---|---|---|---|
| Non-transfected | 1.7 | 1.5 | 2.1 | 2.3 |
| AUG | 5.6 | 5.6 | 1.4 | 3.2 |
| CUG | 22.3 | 20.1 | 7.8 | 7.9 |
| AUA | 25.4 | 22.4 | 9.8 | 9.5 |
| GUG | 26.3 | 26.7 | 9.5 | 9.9 |
| UUG | 27.0 | 29.5 | 11.7 | 15.6 |
| ACG | 32.5 | 34.8 | 13.5 | 18.2 |
| No Acr | 29.0 | 30.0 | 15.5 | 14.0 |

### Example 8: Evaluation of Non-Canonical Start Sites

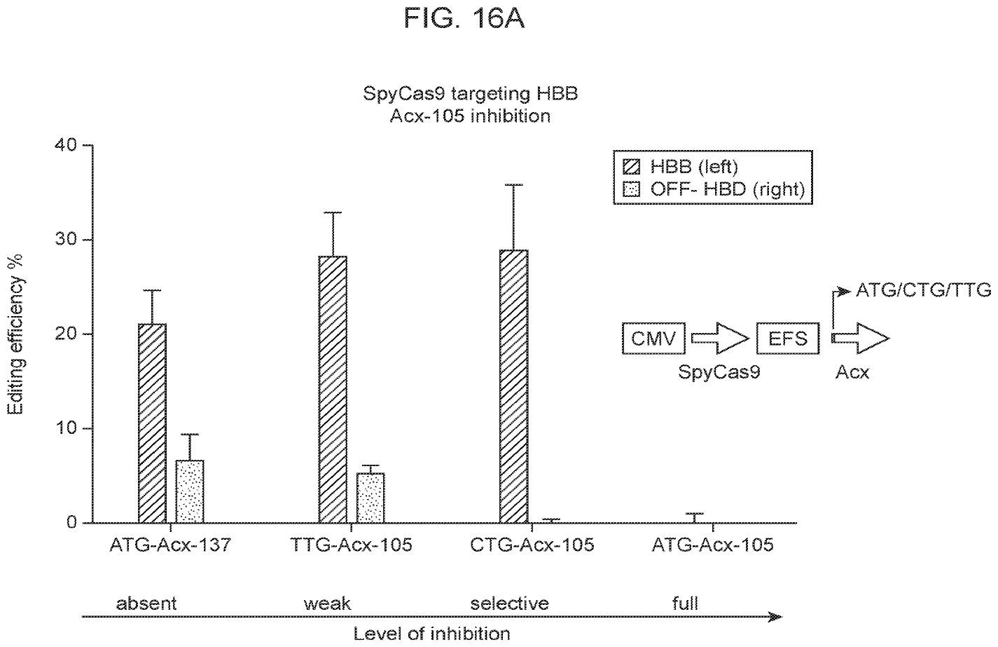

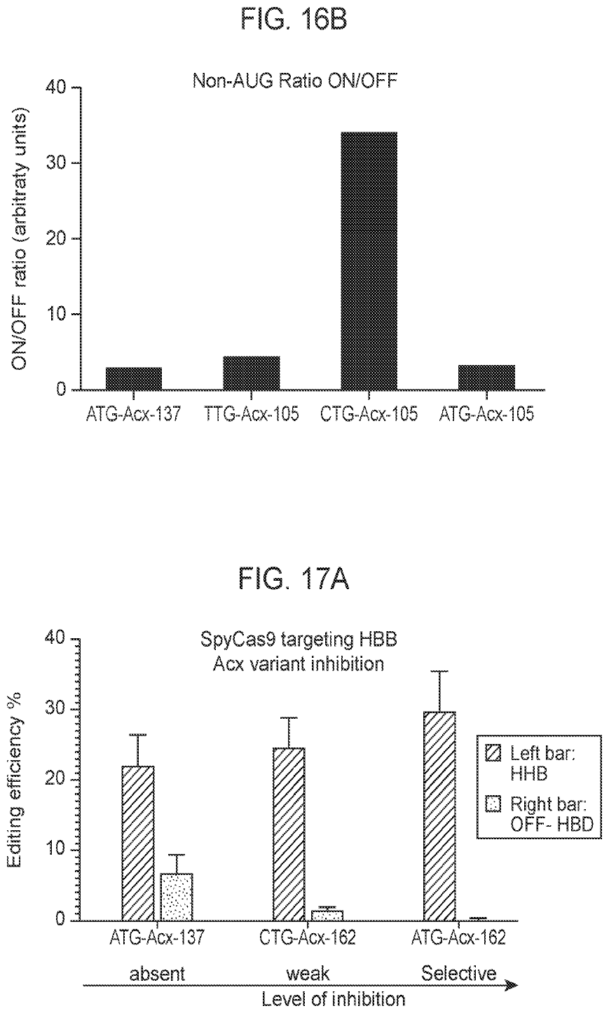

Acx137 and Acx105 were compared for on-target and off-target editing efficiencies as shown in FIG. 16A and FIG. 16B. Acx105 gave similar levels of on-target editing with CTG and TTG start codons. The off-targeting level was significantly lower than on-targeting efficiency in both cases; the TTG start codon provided the lowest level of off-targeting. Under a canonical ATG start codon, the Acx105 abolished nearly all detectable editing.

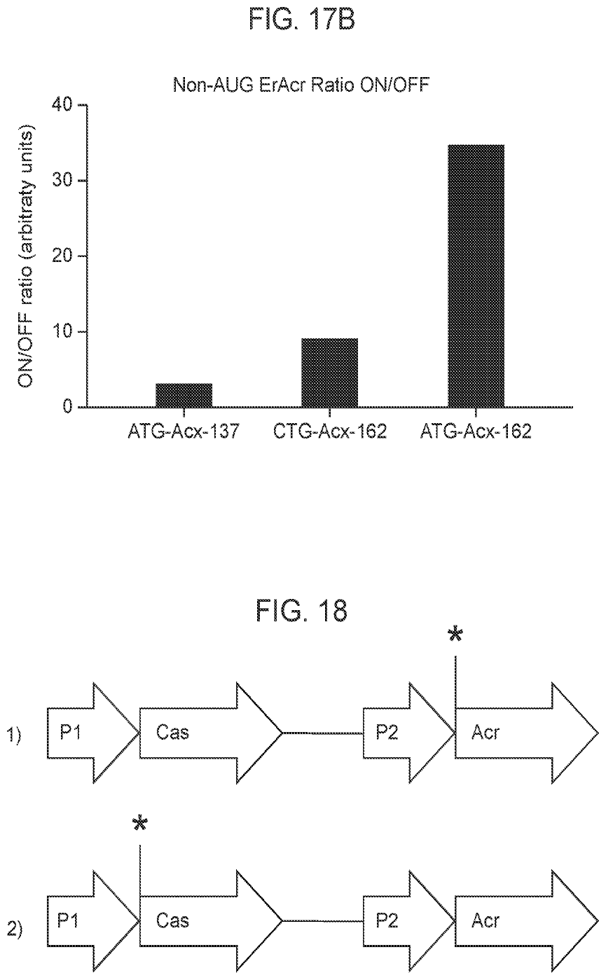

Acx137 and Acx162 were compared for on-target and off-target editing efficiencies as shown in FIG. 17A and FIG. 17B. All three tested constructs had similar levels of on-target editing efficiency. Selectivity for on-targeting versus off-targeting editing was enhanced with the Acx162 constructs.

The comparison of altered start codons and different Acrs shows the tunability of the translational control coupled with varying strength of Acrs. Acx-105 is a strong inhibitor of SpCas9, and exhibits more defined differences in expression with the alternative start codons. Acx-162 is a weaker inhibitor of SpCas9 and shows selective inhibition behavior. Because inhibition is already weak, the dynamic range with the modification of the start codon is reduced as compared with the stronger less selective Acr.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system comprising one or more nucleic acids, wherein the one or more nucleic acids comprise:
(a) a first nucleotide sequence encoding a Cas effector protein;
(b) a second nucleotide sequence encoding an anti-CRISPR protein (Acr protein), wherein the Acr protein is an inhibitor of the Cas effector protein; and
(c) a translational control element that regulates translation of the Cas effector protein or the Acr protein, thereby modulating activity of the Cas effector protein.

2. The system of claim 1, wherein the activity of the Cas effector protein that is modulated is nucleic acid cleavage.

3. The system of claim 1, wherein the activity of the Cas effector protein that is modulated is nucleic acid binding, base editing, transcription modulation, nucleic acid modification, protein modification, and/or or histone modification.

4. The system of any one of claims 1-3, wherein the Acr protein modulates the level or rate of on-target and/or off-target activity of the Cas effector protein.

5. The system of claim 4, wherein the amount of on-target activity of the Cas effector protein is increased by the system as compared with a similar system lacking the translational control element.

6. The system of claim 4, wherein the amount of off-target activity of the Cas effector protein is decreased by the system as compared with a similar system lacking the translational control element.

7. The system of claim 4, wherein the ratio of on-target activity to off-target activity of the Cas effector protein is increased by the system as compared with a similar system lacking the translational control element.

8. The system of any one of claims 1-7, wherein at least one of said one or more nucleic acids is a nucleic acid vector that comprises the first nucleic sequence and the second nucleic sequence.

9. The system of claim 8, wherein the nucleic acid vector is a viral vector.

10. The system of claim 8, wherein the nucleic acid vector comprises an origin of replication.

11. The system of claim 8, wherein the nucleic acid vector is an integrative vector.

12. The system of any one of claims 1-11, further comprising a CRISPR/Cas guide RNA or a nucleic acid that encodes the CRISPR/Cas guide RNA.

13. The system of any one of claims 8-11, wherein the nucleic acid vector encodes a CRISPR/Cas guide RNA.

14. The system of any one of claims 1-13, wherein at least one of said one or more nucleic acids comprises an expression cassette comprising the first nucleic sequence, the second nucleic sequence, and the translational control element, wherein the translational control element is positioned upstream of the first nucleotide sequence.

15. The system of any one of claims 1-13, wherein at least one of said one or more nucleic acids comprises an expression cassette comprising the first nucleic sequence, the second nucleic sequence, and the translational control element, wherein the translational control element is positioned upstream of the second nucleotide sequence.

16. The system of any one of claims 1-13, wherein at least one of said one or more nucleic acids comprises an expression cassette comprising the first nucleic sequence, the second nucleic sequence, and the translational control element, wherein the translational control element is positioned between the first nucleotide sequence and the second nucleotide sequence.

17. The system of claim 16, wherein the translational control element is a sequence that links the first and second nucleotide sequences to one another such that the Cas nuclease and the Acr protein are encoded by a polycistronic sequence.

18. The system of claim 16 or claim 17, wherein the first nucleotide sequence is 5′ to the second nucleotide sequence.

19. The system of claim 16 or claim 17, wherein the second nucleotide sequence is 5′ to the first nucleotide sequence.

20. The system of any one of claims 1-19, wherein the translational control element is an IRES sequence.

21. The system of claim 20, wherein the IRES sequence is selected from the group consisting of EMCV, BIP, CAT-1, c-myc, HCV, VCIP, Apaf-1, mEMCV-1, mEMCV-2, HRV, NRF, FGF-1, KMI1, KM12, (GAAA)16, (PPT19)4, EMCV mutant 5, EMCV mutant 10, EMCV mutant 15, and EMCV mutant 21, and any combination thereof.

22. The system of claim 20 or claim 21, wherein the IRES sequence comprises the sequence set forth in any one of SEQ ID Nos. 139-159.

23. The system of any one of claims 1-19, wherein the translational control element encodes one or more 2A peptides.

24. The system of claim 23, wherein the one or more 2A peptides are selected from the group consisting of: P2A, F2A, E2A, T2A, and any combination thereof.

25. The system of claim 23 or claim 24, wherein at least one of the one or more 2A peptides comprises an amino acid sequence set forth in any one of SEQ ID Nos. 133-138.

26. The system of any one of claims 23-25, wherein the translational control element encodes two or more 2A peptides in tandem.

27. The system of any one of claims 23-25, wherein the translational control element encodes 2, 3, 4, or 5 2A peptides in tandem.

28. The system of any one of claim 1-15, 18 or 19, wherein the translational control element is a non-AUG start codon.

29. The system of claim 28, wherein the non-AUG start codon is at the 5′ end and in-frame with the first nucleotide sequence.

30. The system of claim 29, wherein the first nucleotide sequence does not comprise a native in-frame AUG start codon.

31. The system of claim 28, wherein the non-AUG start codon is at the 5′ end and in-frame with the second nucleotide sequence.

32. The system of claim 31, wherein the second nucleotide sequence does not comprise a native in-frame AUG start codon.

33. The system of any one of claims 28-32, wherein the non-AUG start codon comprises any one of CUG, GUG, ACG, AUA or UUG.

34. The system of any one of claims 1-33, wherein a promoter is operably linked to the first nucleotide sequence.

35. The system of any one of claims 1-33, wherein a promoter is operably linked to the second nucleotide sequence.

36. The system of any one of claims 1-33, wherein a first promoter is operably linked to the first nucleotide sequence and a second promoter is operably linked to the second nucleotide sequence.

37. The system of claim 36, wherein a spacer encoding sequence is positioned 5′ of the first nucleotide sequence and is operably linked to the first promoter, and wherein the translational control element is positioned between the spacer encoding sequence and the first nucleotide sequence.

38. The system of claim 36, wherein a spacer encoding sequence is positioned 5′ of the second nucleotide sequence and is operably linked to the second promoter, and wherein the translational control element is positioned between the spacer encoding sequence and the second nucleotide sequence.

39. The system of any one of claims 1-27, wherein a promoter is operably linked to the first nucleotide sequence, and the first nucleotide sequence is 5′ to the translational control element and the second nucleotide sequence.

40. The system of any one of claims 1-27, wherein a promoter is operably linked to the second nucleotide sequence, and the second nucleotide sequence is 5′ to the translational control element and the first nucleotide sequence.

41. The system of any one of claims 34-35 and 39-40, wherein the promoter is selected from the group consisting of CMV, miniCMV, EFS, chicken β-actin (CBA), human β-actin, herpes simplex virus thymidine kinase hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).

42. The system of any one of claims 36-38, wherein the first promoter and/or the second promoter is selected from the group consisting of CMV, miniCMV, EFS, chicken β-actin (CBA), human β-actin, herpes simplex virus thymidine kinase hybrid promoter CBh, synthetic promoter CAG, human elongation factor-1 alpha (EF1a) EF1a short (EFS), human phosphoglycerate kinase (PGK), mammalian ubiquitin C (UBC), and simian virus 40 (SV40).

43. The system of any one of claims 1-42, wherein the Cas effector protein is selected from the group consisting of a Cas3, a Cas9, a Cas12, and a Cas13.

44. The system of claim 43, wherein the Cas effector protein comprises an amino acid sequence having 70% or more identity with the sequence set forth in any one of SEQ ID Nos. 83-86.

45. The system of any one of claims 1-44, wherein the Acr protein is selected from Table 1 or Table 2.

46. The system of any one of claims 1-44, wherein the Acr protein comprises an amino acid sequence having 70% or more identity with the sequence set forth in any one of SEQ ID Nos. 1-82 and 161.

47. The system of any one of claims 1-44, wherein the Cas effector protein comprises an S. pyogenes Cas9.

48. The system of claim 47, wherein the Acr protein is an AcrIIA2 protein.

49. The system of claim 48, wherein the AcrIIA2 protein comprises an amino acid replacement at one or more positions selected from the group consisting of E12, E16, D22, D23, E25, E26, D38, D40, D60, D61, E63, Y64, D65, D71, E72, V75, E76, D81, E93, D96, 197, D98, D99, L100, E101, D105, E106, D107, E108, M109, K110, S111, G112, N113, Q114, E115, I116, I117, L118, K119, S120, E121, L122, and K123.

50. The system of claim 49, wherein the amino acid replacement at the one or more positions is alanine.

51. The system of claim 47, wherein the Acr protein comprises an AcrIIA4 protein.

52. The system of claim 51, wherein the AcrIIA4 protein comprises an amino acid replacement at one or more positions selected from the group consisting of D5, E9, D14, Y15, T22, D23, N36, D37, G38, N39, E40, Y41, E45, E47, N48, E49, V52, N64, Q65, E66, Y67, E68, D69, E70, E71, E72, F73, Y74, N75, D76, M77, Q78, T79, I80, T81, L82, K83, S84, E85, L86, and N87.

53. The system of claim 52, wherein the replacement at the one or more positions is alanine or arginine.

54. The system of claim 52, wherein the AcrIIA4 protein comprises one or more amino acid replacements selected from the group consisting of D14A, G38A, and N39A.

55. The system of claim 52, wherein the AcrIIA4 protein comprises the amino replacement N39A or the amino acid replacements D14A and G38A.

56. The system of claim 47, wherein the Acr protein is selected from the group consisting of Acx105, Acx137, Acx, 153, Acx162, and Acx164.

57. A cell comprising the system according to any one of claims 1-56.

58. The cell of claim 57, wherein the cell is a mammalian cell or a microorganism.

59. The cell of claim 57, wherein the cell is a human cell.

60. A method of controlling the editing activity of a Cas effector protein comprising:
contacting a target nucleic acid with the system of any one of claims 1-56;
whereby the Cas effector protein mediates one or more edits to a target sequence of the target nucleic acid.

61. The method of claim 60, further comprising measuring the efficacy, level or amount of edits to the target sequence.

62. The method of claim 60, further comprising detecting or identifying one or more edits to the target sequence.

63. The method of any one of claims 60-62, further comprising detecting or identifying one or more edits to a non-target sequence.

64. The method of any one of claims 60-62, further comprising detecting or identifying one or more edits to a non-target sequence.

65. The method of claim 64, further comprising measuring the efficacy, level or amount of edits to the non-target sequence.

66. The method of claim 60, wherein the system provides a ratio of editing the target sequence to editing a non-target sequence is greater than a second ratio of editing the target sequence to editing a non-target sequence provided by the system lacking the Acr protein.

67. The method of claim 60, wherein the system provides a ratio of editing the target sequence to editing a non-target sequence is greater than a second ratio of editing the target sequence to editing a non-target sequence provided by the system lacking the translational control element.

68. The method of claim 60, wherein the system provides an efficiency of editing the target sequence that is greater than an efficiency of editing a non-target sequence.

69. The method of claim 63, wherein the target sequence and the non-target sequence share greater than 90% but less than 100% sequence identity.

70. The method of claim 63 or claim 64, wherein the efficiency of editing the target sequence is at least 2×, 4×, 5×, 10×, 12×, 15×, 20×, 25×, 30×, 35× greater than the efficiency of editing a non-target sequence.

71. The method of claim 63 or claim 64, wherein the ratio of editing the target sequence to editing the non-target sequence is at least 2, 4, 5, 10, 12, 15, 20, 25, 30, 35 or greater than 35.

72. The method of any one of claims 60-71, wherein the target nucleic acid is in a cell.

73. The method of claim 72, wherein the cell is a mammalian cell or a microorganism.

74. The method of claim 72, wherein the cell is a human cell.

75. The method of any one of claims 72-74, wherein the contacting step comprises introducing the system into the cell.

76. The method of any one of claims 60-71, wherein the target nucleic acid is not inside of a cell.

77. The method of claim 76, wherein the method is an in vitro assay.

78. The method of claim 77, wherein the in vitro assay is a diagnostic assay.
